(19) United States
(12) Patent Application Publication
(10) Pub. No.: US 2009/0162329 A1
(43) Pub. Date: Jun. 25, 2009

(54) COMPOSITIONS COMPRISING HDAC INHIBITORS AND METHODS OF THEIR USE IN RESTORING STEM CELL FUNCTION AND PREVENTING HEART FAILURE

(76) Inventors: Piero Anversa, Boston, MA (US); Annarosa Leri, Boston, MA (US); Jan Kajstura, Brookline, MA (US)

Correspondence Address: COOLEY GODWARD KRONISH LLP;ATTN: Patent Group, Suite 1100, 777 - 6th Street, NW, WASHINGTON, DC, 20001, US

(21) Appl. No.: 12/325,816

(22) Filed: Dec. 1, 2008

Related U.S. Application Data
(60) Provisional application No. 60/991,663, filed on Nov. 30, 2007.

(51) Int. Cl. A61K 35/12; C12N 5/08

(52) U.S. Cl. 424/93.7; 435/366; 435/377

(57) ABSTRACT

The invention provides compositions of histone deacetylase (HDAC) inhibitors and progenitor cells useful for treating heart failure in a subject. The invention also provides methods of restoring progenitor cell function to aged progenitor cells and methods for enhancing progenitor cell proliferation and/or differentiation using HDAC inhibitors.

### CROSS REFERENCE TO RELATED APPLICATIONS

This application claims the benefit of U.S. Provisional Application No. 60/991,663, filed Nov. 30, 2007, which is herein incorporated by reference in its entirety.

### FIELD OF THE INVENTION

The present invention relates generally to the field of cardiology, and more particularly relates to the use of histone deacetylase inhibitors (HDAC) for restoring adult progenitor cell function. The invention also relates to methods of using compositions comprising histone deacetylase inhibitors and adult progenitor cells for treating heart failure.

### BACKGROUND OF THE INVENTION

The recognition that the adult human heart contains a pool of resident c-kit-positive cardiac progenitor cells (PCs) has raised the opportunity to reconstitute the decompensated failing heart (1). Cardiac PCs can be isolated from biopsy samples and, following their expansion in vitro, can be transplanted into the same patient to regenerate scarred myocardium (1-4). Alternatively, portions of damaged myocardium can be restored by cytokine activation of resident PCs (5-10) which migrate to the site of injury where they subsequently form functionally competent myocardium (6, 7). These two therapeutic modalities are not mutually exclusive but complement each other. Encouraging experimental results with these approaches (1-15), however, have left unanswered the question whether cardiac PCs can reconstitute the vascular framework and reestablish blood flow to the poorly perfused myocardium. This possibility would change the current target of cell therapy: from the attempt to repair the damaged heart to the effort to prevent ischemic myocardial injury.

Several reports in the literature recognize a cardiac PC that forms substantial quantities of cardiomyocytes after infarction (1, 6, 7, 11). Although this work has been successful, to prevent ischemic myocardial damage acutely and the development of an ischemic myopathy chronically, it is desirable to identify a PC which is capable of restoring the integrity of injured coronary vessels and/or creating de novo conductive coronary arteries and their distal branches. To achieve this goal, a profound understanding of the biology of resident PCs is required and must determine whether this PC pool includes a class of cells which have powerful vasculogenic properties. Identification of a coronary vascular PC able to differentiate predominantly into smooth muscle cells (SMCs) and endothelial cells (ECs) would suggest that the heart possesses the inherent ability to create the various portions of the coronary circulation. Damaged large coronary arteries could be replaced by newly formed vessels and rarefaction of resistance coronary arterioles and capillary structures could be corrected by expansion of the cardiac microcirculation. If this is possible, cell therapy would be employed to interfere with ischemic injury, the prevailing cause of human heart failure. Prevention may supersede the need for myocardial regeneration.

In the multipotent state of PCs, genes that are required in the differentiated progeny are transiently held in a repressed state by histone modifications, which are highly flexible and easily reversed when the expression of these genes is needed (109, 112-114). Conversely, genes that are associated with sternness are stably maintained in an active state (115-117). With differentiation, genes that are crucial for multipotency are silenced through histone modifications and DNA methylation (118-121). In PC commitment, the acquisition of a specific lineage imposes the upregulation of a selected network of genes and the silencing of all other differentiation programs within the cells (122). For example, a neural stem cell that makes the decision to become a neuron has to inhibit the molecular program associated with glial formation (122). The recognition that stem cells retain a considerable degree of developmental plasticity has made apparent that gene silencing is more complex than originally thought (68, 90-92, 123). It would be desirable to modulate the expression of genes related to stem cell function in PC populations.

Epigenetic changes, which are heritable during cell division, are implicated in human aging and disease, suggesting that myocardial aging and heart failure may lead to epigenetic lesions of PCs. Epigenetic abnormalities may affect the phenotypic plasticity of PCs and thereby their ability to respond to alterations in the cardiac microenvironment which occur with aging and chronic heart failure. In both cases, telomeric shortening takes place in human cardiac PCs and telomere attrition may be coupled with the expression of senescence-associated genes which may inhibit cell replication and trigger cell death. Thus, there is a need in the art for methods of preserving PC function, particularly in the aging heart, to sustain the ability of the heart to repair itself.

### SUMMARY OF THE INVENTION

The present invention discloses compositions and methods for repressing and activating genes that regulate sternness and commitment of different classes of progenitors cells, such as vascular progenitor cells (VPCs), myocyte progenitor cells (MPCs), and bone marrow progenitor cells (BMPCs). In one embodiment, a composition of the invention comprises a histone deacetylase (HDAC) inhibitor and one or more types of human progenitor cells. The one or more human progenitor cells may be human VPCs, MPCs, BMPCs, or combinations thereof. In another embodiment, said HDAC inhibitor targets class I or class II HDAC enzymes. In another embodiment, said HDAC inhibitor is an inhibitory RNA molecule (e.g. siRNA or shRNA) targeted to a class I or class II HDAC enzyme.

The present invention also provides a method of enhancing progenitor cell proliferation. In one embodiment, the method comprises exposing human adult progenitor cells to one or more HDAC inhibitors, wherein said progenitor cells exhibit enhanced proliferation as compared to progenitor cells not exposed to the one or more HDAC inhibitors. In preferred embodiments, said human adult progenitor cells are VPCs, MPCs, or BMPCs. In some embodiments, the one or more HDAC inhibitors target a class I and/or class II HDAC enzyme.

The present invention also includes a method of enhancing progenitor cell differentiation. In one embodiment, the method comprises exposing human adult progenitor cells to one or more HDAC inhibitors, wherein said progenitor cells exhibit enhanced differentiation as compared to progenitor cells not exposed to the one or more HDAC inhibitors. In preferred embodiments, said human adult progenitor cells are VPCs, MPCs, or BMPCs. In some embodiments, the one or more HDAC inhibitors target a class I and/or class II HDAC enzyme.

The present invention encompasses a method of restoring progenitor cell function to aged adult progenitor cells, wherein said method comprises exposing said aged progenitor cells to one or more HDAC inhibitors, wherein said progenitor cells exhibit increased expression of at least one stem cell related gene as compared to aged progenitor cells not exposed to the one or more HDAC inhibitors. In one embodiment, said stem cell related gene is Oct4. In another embodiment, said stem cell related gene is Nanog. In some embodiments, the aged progenitor cells are isolated from a subject suffering from heart failure.

The present invention also provides a method of treating heart failure in a subject in need thereof. In one embodiment, the method comprises isolating adult progenitor cells from a tissue specimen from the subject; exposing said isolated progenitor cells to one or more HDAC inhibitors; and administering said treated progenitor cells to the subject's heart, wherein said progenitor cells generate new coronary vessels and myocardium, thereby improving cardiac function. In preferred embodiments, said adult progenitor cells are VPCs, MPCs, or BMPCs. In some embodiments, the one or more HDAC inhibitors target a class I and/or class II HDAC enzyme. At least one symptom of heart failure may be reduced in the subject following administration of the treated progenitor cells.

### BRIEF DESCRIPTION OF THE DRAWINGS

FIG. 1. Vascular and myocardial niches. A: Transverse section of an epicardial human coronary artery in which the area in the rectangle is shown at higher magnification in panels B and C: One c-kit-positive (B: green, arrow) KDR-positive (C: higher magnification; white, arrow) VPC is present within the adventitia. N-cadherin (yellow, arrowheads) is located between the c-kit-positive KDR-positive VPC and a cell labeled by α-smooth muscle actin (α-SMA: red), most likely a myofibroblast. D-G: Small human coronary arterioles in which, in both cases, one c-kit-positive (D and E) KDR-positive VPC (F and G: higher magnification; arrows), is present within the SMC layer (α-SMA: red); connexin 45 (Cx45) is distributed between the VPCs and SMCs (F and G, arrowheads). H: Tangential section of epicardial human coronary artery; myocytes are labeled by α-sarcomeric actin (α-SA, white) and the adventitia by collagen (yellow). The three areas in the rectangles are shown at higher magnification in panels I-N: one group of 6 and two of 3 c-kit-positive (I, K M: green) KDR-positive (J, L, N: white) VPCs are present within the adventitia. Connexin 43 (Cx43:red) is expressed between VPCs and fibroblasts (procollagen, light blue). O: Human myocardium containing 14 c-kit-positive MPCs (green). The arrows define the two areas shown at higher magnification in the adjacent panels. Cx43 (white dots) and N-cadherin (magenta dots) are present between two MPCs, and between MPCs and myocytes (α-SA, red) or MPCs and fibroblasts (procollagen, light blue). The c-kit-positive cells are negative for KDR (not shown).

FIG. 2. Surface epitopes of VPCs and MPCs. VPCs and MPCs were isolated from human myocardial samples and expanded in vitro. A. VPCs were c-kit and KDR positive and negative for hematopoietic markers (CD34, CD45, CD133, cocktail of lineage epitopes) and α-sarcomeric actin (α-SA) and expressed at very low levels CD31 and TGF-β1 receptor. Immunocytochemically, VPCs were c-kit-positive (green) and KDR positive (red) consistent with the FACS data. B. MPCs were c-kit-positive and KDR-negative. MPCs were negative for hematopoietic markers (CD34, CD45, CD133, cocktail of lineage epitopes), CD31 and TGF-β1 receptor and expressed at very low level α-SA. Immunocytochemically, MPCs were c-kit-positive (green) and KDR-negative consistent with the FACS data.

FIG. 3. VPCs and MPCs are self-renewing, clonogenic and multipotent. Clones derived from single VPCs isolated from human coronary vessels (A, B) and single MPCs isolated from human myocardial samples (C-E). VPC clones (A) are positive for c-kit (green), KDR (red) and both c-kit and KDR (yellow). Human VPC (B) and MPC (C) clones are shown by phase contrast microscopy. D: From a single MPC, a multicellular clone was developed in 9 days. MPC clones are positive for c-kit (green) and negative for KDR (not shown). E: MPCs in the clone are positive for c-kit (green) and negative for bone marrow cell markers. Bone marrow cells were used as positive controls for CD34, CD45, CD133 and lineage epitopes. F: VPCs form 3.3-fold more SMCs (*) and 2.5-fold more ECs (*) than MPCs while MPCs form 3.5-fold more myocytes (*) than VPCs.

FIG. 4. VPCs generate large coronary vessels. A: A critical stenosis of the LAD was created and human EGFP-positive VPCs were injected around the stenotic artery. Thirty days after coronary constriction and cell implantation, a large developing artery (A: diameter=˜0.56 mm) was detected in proximity of the stenotic vessel. The new vessel was identified by α-SMA labeling (A: red), EGFP expression (B: green) and the human-specific sequence Alu (C: white). Co-expression of α-SMA and EGFP (D: yellow).

FIG. 5. Myocardial regeneration. A, B: Human myocardium (arrowheads) in a treated infarcted mouse at 21 days (A) and treated infarcted rat at 14 days (B). New myocytes are positive for α-SA (red) The human origin of the myocardium was confirmed by the detection of human DNA sequences for Alu in nuclei (green); BrdU was given throughout the experiment to label newly formed myocytes (B: upper panel, white).

FIG. 6. Cardiac chimerism. Female patient with chronic lymphocytic leukemia who died 26 days after sex mismatched bone marrow transplantation. Three Y-chr positive cells (green dots, arrows) are present in the myocardial interstitium (A). Two small developing male myocytes are also present (B, C: α-SA, red; arrows).

FIG. 7. VPCs and MPCs in the fetal heart. Human fetal heart at ˜17-21 weeks of gestation: 3 c-kit-positive (A: green) KDR-positive (B: red) VPCs are present in the ventricular myocardium. Similarly, 3 c-kit-positive (C: green) KDR-negative (not shown) MPCs are shown. The junctional protein Cx43 (white dots) was detected at the interface between MPCs and developing myocytes (arrows). D: One c-kit-positive (left panel, green) KDR-negative (not shown) MPC expresses α-SA (central panel, red). The right panel shows the merge of the left and right panels. This suggests a linear relationship between MPCs and myocyte formation in the developing human heart.

FIG. 8. PC Stemness and commitment. Oct4 and Nanog may regulate the undifferentiated state of embryonic-fetal precursors and adult PCs. Downregulation of Oct4 and Nanog together with the surface epitopes of PCs leads to cell commitment. The acquisition of specific lineages is conditioned by the expression of myocyte (Nkx2.5, MEF2), EC (eNOS, e-Cadh) and SMC (SRF, GATA6) genes.

FIG. 9. Histone code. The nucleosome consists of DNA and four pairs of histones. Post-translational modifications of histones include methylation (Me), acetylation (Ac), ubiquitination (Ub), sumoylation (Su) and phosphorylation (P) and condition the formation of euchromatin and heterochromatin. TF, transcription factors.

FIG. 10. Schematic showing pathway and genes that may be involved in the regulation of stemness and commitment of progenitor cells.

FIG. 11. DNA methylation of eNOS promoter. Methylated and unmethylated CpG dinucleotides in the eNOS promoter were studied in human cell populations. Methylation was apparent in the three PC classes: EPCs (adult donors), mesangioblasts (children) and CD34-positive BMPCs (adult donors). CpG dinucleotides were unmethylated in cells committed to the endothelial lineage: HUVEC and microvascular ECs (MVEC).

FIG. 12. Histone methylation in human VPCs and MPCs. VPCs and MPCs show a bivalent chromatin configuration. H3K27me3, H3K4me2 and H3K9me2 were detected by Western blotting (A-C) and immunocytochemistry and confocal microscopy (D-H). H3K27me3 (D: red), H3K4me2 (E, F: red) and H3K9me2 (G, H: red) are localized in the nuclei of VPCs and MPCs. VPCs express c-kit (D, E, G, green) and KDR (D, E, G, white). MPCs express c-kit (F, H, green) and are negative for KDR (not shown).

FIG. 13. Histone acetylation in VPCs, MPCs and ESCs. VPCs and MPCs show H3K9Ac and H3K14Ac by Western blotting (A, B) and immunocytochemistry (C, D). H3K9Ac (C, D: red) is present in nuclei of VPCs (C) and MPCs (D). VPCs express c-kit (C: green) and KDR (C: white). MPCs express c-kit (D: green) and are negative for KDR (not shown). E: Chromatin immunoprecipitation (ChIP) assay in mouse ESCs. Arrow indicates the position of the PCR product representing the Oct4 promoter. DNA templates were obtained from a protein-DNA complex immunoprecipitated with H3K9Ac-specific antibody (Ab). Input, DNA quantity used. Neg, negative control with IgG only.

FIG. 14. Epigenetics of PCs. Chromatin structure predictive of a multipotent state carries a bivalent configuration of histones characterized by activating and inactivating marks in the same or adjacent nucleosomes. Activating marks include acetylation of histones H3 and H4 at lysine residues and methylation of histone H3 at lysine 4. Inactivating marks include methylation of histone H3 at lysine residues and DNA methylation.

FIG. 15. Histone methylation in VPCs, MPCs and ESCs. A: H3K79me2 is present in MPCs and absent in VPCs. B: Shear stress (SS) induces a 4-5-fold increase in H3K79me2 in mouse ESCs. Trichostatin A (TSA) reduces the overall methylation level of histone H3. Equal loading is determined on the basis of histone H1.

FIG. 16. Schematic depicting the classification of histone deacetylases (HDACs).

FIG. 17. HDACs in human cardiac PCs. VPCs and MPCs express HDAC2-5 and HDAC7 by Western blotting (A-E). HDAC3 and HDAC4 form a complex in MPCs (F). Cell lysates were immunoprecipitated with an antibody against HDAC3 and Western blotting was performed with HDAC4-antibody. By immunocytochemistry, HDAC4 (G, H: red) shows a nuclear and cytoplasmic localization in VPCs (G) and a nuclear distribution only in MPCs (H). HDAC7 is distributed in the nucleus and cytoplasm in MPCs (I: yellow). VPCs express c-kit (G: green) and KDR (G: white). MPCs express c-kit (H, I: green) and are negative for KDR (not shown).

FIG. 18. HDACs in mouse ESCs. A, B: In the presence of LIF, HDAC4 (A: red, mid-panels) and HDAC7 (B: white, mid-panels) show a diffuse distribution in ESCs. One hour after LIF removal (1 h), both HDAC isozymes are restricted to the nucleus. At 3 (3 h) and 6 hours (6 h), HDAC4 and HDAC7 are present in both nucleus and cytoplasm. DAPI staining of nuclei, blue (upper panels). C: The prevailing nuclear localization of HDAC4 at 1 hour after LIF removal was confirmed by immunoprecipitation and Western blotting of nuclear protein lysates. D: HDAC3 and HDAC4 form a complex in ESCs. Cell lysates were immunoprecipitated with an antibody against HDAC3 and Western blotting was performed first with HDAC4-antibody and subsequently with HDAC3-antibody. E: The activity of HDAC was measured by employing acetylated H4 as substrate. Enzymatic nuclear HDAC activity peaks at 1 hour after LIF removal.

FIG. 19. HDACs in HUVEC. A: HUVEC were incubated with siRNA oligonucleotides directed against individual HDAC isozymes. At 24 hours, mRNA expression of HDAC4, HDAC5, HDAC7 and HDAC9 was selectively suppressed. B: Capillary-sprout formation from three-dimensional spheroids was not affected by suppression of HDAC4 and HDAC9. HDAC5-siRNA increased sprout length while HDAC7-siRNA decreased this process. C: HUVEC migration was enhanced by HDAC4-siRNA and HDAC5-siRNA and decreased by HDAC7-siRNA and HDAC9-siRNA. D: Transfection of HUVEC with mutated HDAC markedly reduced sprout length. This construct has mutations in serine 259 and 498 opposing HDAC5 phosphorylation and promoting its nuclear sequestration. E, F: Sprout formation was determined by Matrigel assay and plug implantation subcutaneously. HDAC5-siRNA increased hemoglobin concentration (E; Hb) and the number of invaded cells (F) in the Matrigel plugs.

FIG. 20. Stem cell division. A: Human myocardium containing 6 MPCs (c-kit: green) one of which is in mitosis (phospho-H3: magenta). Alpha-adaptin (white) is uniformly distributed in the dividing cell (symmetric division). B: Human myocardium containing 5 MPCs one of which is in mitosis. Numb (yellow) is not uniformly localized in the dividing cell (asymmetric division). C-D: Human MPCs in culture. The dividing MPC(C: arrow, left panel) is shown at higher magnification in the right panel of C: Chromosomes are in metaphase and alpha-adaptin is uniformly distributed in the dividing cell (symmetric division). The dividing MPC (D: arrows, left panel) is shown at higher magnification in the right panel of D: Chromosomes are in late anaphase initial telophase and alpha-adaptin is not uniformly distributed in the dividing cell (asymmetric division).

FIG. 21. Gene expression profile of VPCs and MPCs. The stemness-related genes (left) that are upregulated in MPCs versus VPCs include Wnt1, Notch1 and Sox1. Oct4 is similarly expressed in VPCs and MPCs (not shown). The lineage-related genes (right) that are more expressed in MPCs than VPCs include Nkx2.5, Tbx1, Hoxa9 and GATA1 and those that are more expressed in VPCs than MPCs include multimerin (Mmrn1), VCAM, eNOS and vWf.

FIG. 22. SIRT1 and vessel growth. A: Transfection with specific siRNAs induces the suppression of mRNAs of SIRT1, SIRT2, SIRT3 and SIRT5 in HUVEC. B: Sprout formation from individual siRNA-transfect spheroids was affected by SIRT1-siRNA. C: Angiogenesis and Matrigel assays in vitro in the presence of SIRT1-siRNA or scrambled control. D: Lateral views of the vasculature in wild-type and in SIRT1-knock-down (ATG morpholino and SB morpholino) zebrafish embryos. Arrows point to defects in the formation of intersomitic vessels. E: Hemorrhages (white arrows) and pericardial swelling (black arrows) are visible in SIRT1 knock down zebrafish. F: After hind limb ischemia and perfusion, blood flow is significantly reduced in mice with a conditional EC-specific deletion of SIRT1. G: SIRT1 and Foxo1 form a complex in HUVEC. H: Acetylation of Foxo1 in HUVEC in the presence and absence of the SIRT1 inhibitor nicotinamide (NAM). I: Acetylation of Foxo1 in HUVEC in the presence and absence of the SIRT1 inhibitor nicotinamide (NAM), acetyltransferase p300 and SIRT1-siRNA. J: VPCs and MPCs express SIRT1. The higher level of expression of SIRT1 in lane 3 corresponds to MPCs obtained from a patient 35 years of age.

FIG. 23. Effect of HDAC inhibitors on ESC differentiation. In the presence of LIF (+LIF), undifferentiated ESCs do not express the vascular marker flk1 and the neuronal marker nestin. Following LIF removal (−LIF), the addition of trichostatin (TSA, class I and II HDAC inhibitor) or MC1568 (class II HDAC inhibitor) leads to selective expression of nestin (red) and neuronal differentiation of ESCs. Conversely, treatment of ESCs with MS27-275 (MS, class I HDAC inhibitor) promotes the preferential differentiation of ESCs into cardiovascular lineages (flk1, green). DAPI, blue.

FIG. 24. Myocardial regeneration. A-D: Infarcted rat hearts injected with clonogenic MPCs 20 days after infarction. The area included in the rectangle (A) is shown at higher magnification in B. Arrowheads delimit the area of regenerated myocardium. Two other examples of myocardial regeneration are shown in panels C and D; ˜40% of the scar was replaced by functional myocardium as demonstrated by the reappearance of contraction in the infarcted region of the wall. Panels E and F illustrate by echocardiography the non-contracting infarcted region of the wall (E) and the same region after cell treatment (F). G: Improvement in ventricular function of infarcted treated hearts (MI-T). Panels H and I illustrate regenerated myocytes in the aging heart of Fischer 344 rats. When myocytes are formed in closed proximity to differentiated cells they assume the adult phenotype (H) while in damage foci they resemble fetal-neonatal myocytes (I). Bars=10 μm.

FIG. 25. Schematic depicting experimental protocol for treating isolated human VPCs, MPCs, or BMPCs with a histone deacetylase (HDAC) inhibitor in vitro for subsequent administration to the heart.

### DETAILED DESCRIPTION OF THE INVENTION

As used herein, “autologous” refers to something that is derived or transferred from the same individual's body (i.e., autologous blood donation; an autologous bone marrow transplant).

As used herein, “allogeneic” refers to something that is genetically different although belonging to or obtained from the same species (e.g., allogeneic tissue grafts or organ transplants).

As used herein, “stem cells” are used interchangeably with “progenitor cells” and refer to cells that have the ability to renew themselves through mitosis as well as differentiate into various specialized cell types. The stem cells used in the invention are somatic stem cells, such as bone marrow or cardiac stem cells or progenitor cells. “Vascular progenitor cells” or VPCs are a subset of adult cardiac stem cells that are c-kit positive and KDR (e.g. flk1) positive, which generate predominantly endothelial cells and smooth muscle cells. “Myocyte progenitor cells” or MPCs are a subset of adult cardiac stem cells that are c-kit positive and KDR (e.g. flk1) negative, which generate cardiomyocytes predominantly.

As used herein, “adult” stem cells refers to stem cells that are not embryonic in origin nor derived from embryos or fetal tissue.

Stem cells (e.g. progenitor cells) employed in the invention are advantageously selected to be lineage negative. The term “lineage negative” is known to one skilled in the art as meaning the cell does not express antigens characteristic of specific cell lineages. For example, bone marrow progenitor cells (BMPCs) do not express any of the hematopoietic lineage markers, such as CD3, CD20, CD33, CD14, and CD15. And, it is advantageous that the lineage negative stem cells are selected to be c-kit positive. The term “c-kit” is known to one skilled in the art as being a receptor which is known to be present on the surface of stem cells, and which is routinely utilized in the process of identifying and separating stem cells from other surrounding cells.

As used herein, the term “cytokine” is used interchangeably with “growth factor” and refers to peptides or proteins that bind receptors on cell surfaces and initiate signaling cascades thus influencing cellular processes. The terms “cytokine” and “growth factor” encompass functional variants of the native cytokine or growth factor. A functional variant of the cytokine or growth factor would retain the ability to activate its corresponding receptor. Variants can include amino acid substitutions, insertions, deletions, alternative splice variants, or fragments of the native protein. The term “variant” with respect to a polypeptide refers to an amino acid sequence that is altered by one or more amino acids with respect to a reference sequence. The variant can have “conservative” changes, wherein a substituted amino acid has similar structural or chemical properties, e.g., replacement of leucine with isoleucine. Alternatively, a variant can have “nonconservative” changes, e.g., replacement of a glycine with a tryptophan. Analogous minor variations can also include amino acid deletion or insertion, or both. Guidance in determining which amino acid residues can be substituted, inserted, or deleted without eliminating biological activity can be found using computer programs well known in the art, for example, DNASTAR software.

As used herein, the term “histone deacetylase inhibitor” or “HDAC inhibitor” refers to a compound which is capable of interacting with a histone deacetylase and inhibiting its enzymatic activity. “Inhibiting histone deacetylase enzymatic activity” means reducing the ability of a histone deacetylase to remove an acetyl group from a histone. In some preferred embodiments, such reduction of histone deacetylase activity is at least about 50%, more preferably at least about 75%, and still more preferably at least about 90%. In other preferred embodiments, histone deacetylase activity is reduced by at least 95% and more preferably by at least 99%. The histone deacetylase inhibitor may be any molecule that effects a reduction in the activity of a histone deacetylase. This includes proteins, peptides, DNA molecules (including antisense), RNA molecules (including RNAi and antisense) and small molecules.

As used herein “damaged myocardium” refers to myocardial cells which have been exposed to ischemic conditions. These ischemic conditions may be caused by a myocardial infarction, or other cardiovascular disease or related complaint. The lack of oxygen causes the death of the cells in the surrounding area, leaving an infarct, which will eventually scar.

As used herein, “patient” or “subject” may encompass any vertebrate including but not limited to humans, mammals, reptiles, amphibians and fish. However, advantageously, the patient or subject is a mammal such as a human, or a mammal such as a domesticated mammal, e.g., dog, cat, horse, and the like, or production mammal, e.g., cow, sheep, pig, and the like.

The pharmaceutical compositions of the present invention may be used as therapeutic agents—i.e. in therapy applications. As herein, the terms “treatment” and “therapy” include curative effects, alleviation effects, and prophylactic effects. In certain embodiments, a therapeutically effective dose of progenitor cells is applied, delivered, or administered to the heart or implanted into the heart in combination with an HDAC inhibitor. In other embodiments, a therapeutically effective dose of progenitor cells is treated with an HDAC inhibitor prior to administration to the heart. An effective dose or amount is an amount sufficient to effect a beneficial or desired clinical result. Said dose could be administered in one or more administrations.

Mention is made of the following related pending patent applications:

U.S. Application Publication No. 2003/0054973, filed Jun. 5, 2002, which is herein incorporated by reference in its entirety, discloses methods, compositions, and kits for repairing damaged myocardium and/or myocardial cells including the administration cytokines.

U.S. Application Publication No. 2006/0239983, filed Feb. 16, 2006, which is herein incorporated by reference in its entirety, discloses methods, compositions, and kits for repairing damaged myocardium and/or myocardial cells including the administration of cytokines and/or adult stem cells as well as methods and compositions for the development of large arteries and vessels. The application also discloses methods and media for the growth, expansion, and activation of human cardiac stem cells.

The inventors have recently discovered that the human heart possesses two categories of progenitor cells (PCs): coronary vascular progenitor cells (VPCs) and myocyte progenitor cells (MPCs). See, e.g., U.S. Provisional Application No. 60/991,515, filed Nov. 30, 2007, which is herein incorporated by reference in its entirety. VPCs, which are c-kit positive and KDR (e.g. flk1) positive, are nested in vascular niches located in the coronary circulation and MPCs, which are c-kit positive and KDR (e.g. flk1) negative, are clustered in myocardial niches distributed in the muscle compartment. In vitro, VPCs are self-renewing, clonogenic and multipotent and differentiate predominantly into vascular endothelial cells (ECs) and smooth muscle cells (SMCs) and to a limited extent into myocytes. MPCs are also self-renewing, clonogenic and multipotent but differentiate prevalently into myocytes and to a much lesser degree into ECs and SMCs. Functionally, VPCs generate in vivo the various portions of the coronary vasculature from large conductive coronary arteries to capillary structures. Additionally, they can form a small number of cardiomyocytes. Conversely, MPCs generate in vivo large quantities of cardiomyocytes and small amounts of resistance arterioles and capillaries.

Epigenetic mechanisms may be responsible for the molecular identity and functional behavior of PCs. Epigenetics corresponds to genomic information heritable during cell division other than the DNA sequence itself. The phenotypic plasticity of cells with essentially identical DNA sequences may be modulated by the epigenome. Epigenetic mechanisms are implicated in gene activation and silencing at the level of transcription. They include post-translational modifications of histones—acetylation, methylation, phosphorylation—DNA methylation of CpG nucleotides, ATP-dependent chromatin remodeling, exchange of histones and histone variants, and small RNA molecules. Together, epigenetic mechanisms condition the packaging of DNA and histones into highly condensed heterochromatin or loose unfolded euchromatin. While euchromatin is permissive, heterochromatin is resistant to transcriptional activation. Typically, epigenetics is implicated in the regulation of pluripotency and differentiation of embryonic stem cells by preserving the uncommitted state or promoting the acquisition of specific cell lineages.

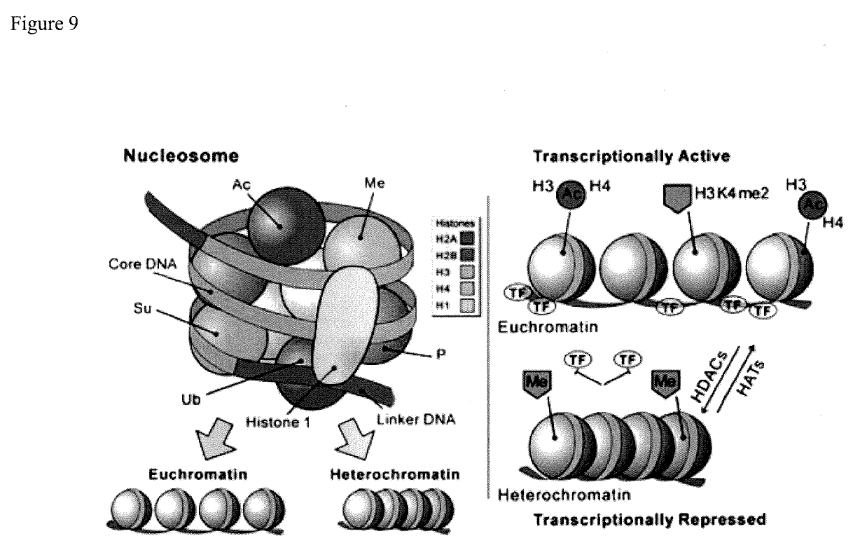

Epigenetics of selective genes are considered the critical determinants of stemness and lineage commitment of PCs including bone marrow progenitor cell (BMPC) transdifferentiation. Studies in mouse embryonic stem cells (ESCs), neural stem cells and hematopoietic stem cells (HSCs) have shown that the control of self-renewal, multipotentiality and commitment occurs largely at the transcriptional level (101-105). However, it is becoming increasingly clear that epigenetic mechanisms play also an important role in stem cell function (106-108). Epigenetic mechanisms comprise short-term flexible modifications of chromatin which can be removed before a cell divides or within a few cell divisions (109). Conversely, long-term stable epigenetic changes can be maintained for many divisions. These modifications constitute the histone code (110) which is conditioned by the peculiar organization of the eukaryotic DNA in nucleosomes (FIG. 9). Post-translational modifications of histone tails constitute the nucleosome code (111) and determine the formation of regions of euchromatin (transcriptionally active) and heterochromatin (transcriptionally repressed) (108). Thus, histone modifications—methylation, acetylation, ubiquitination, sumoylation, phosphorylation—lead to either gene activation or silencing.

The inventors have discovered that the undifferentiated and differentiated states of VPCs, MPCs and BMPCs may be epigenetically regulated by DNA methylation, and acetylation and methylation of lysine residues of core histones. Thus, one aspect of the present invention is to provide methods of preserving the stemness of progenitor cells or guide progenitor cell differentiation by modulating DNA methylation or acetylation and methylation of histone proteins.

DNA methylation occurs on cytosine at CpG dinucleotides which are asymmetrically distributed into CpG poor regions and dense regions termed CpG islands (124). These CpG islands are mostly located in gene promoters and their methylation results in repression of transcription (125). However, a low density of methylated CpG induces weak silencing that can be overcome by strong gene activators (16, 127). DNA methylation interferes with gene transcription directly by opposing the binding of transcription factors to their specific promoter sequences or indirectly by favoring the association of repressor protein complexes with gene promoters (124). Conversely, the expression of specific genes is mediated by demethylation of the corresponding regulatory regions (128, 129). Therefore, repression and activation of genes that regulate stemness and commitment of VPCs, MPCs and BMPCs may be conditioned, respectively, by methylation and demethylation of DNA sequences at their promoter regions.

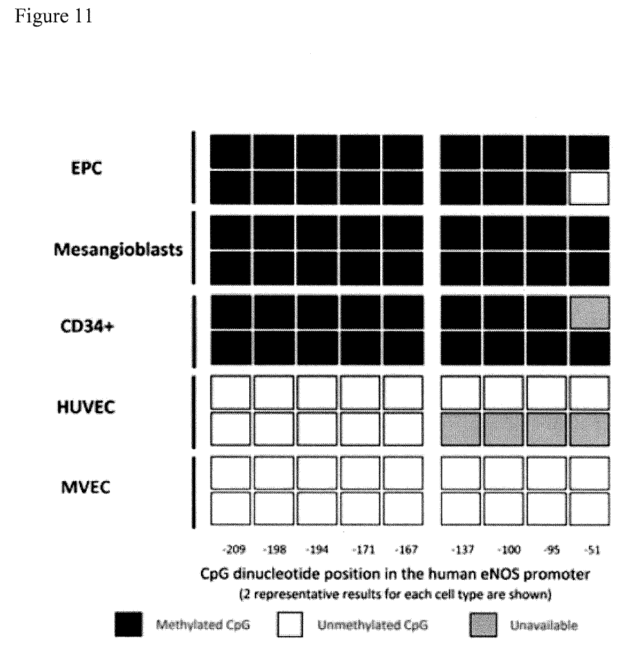

Recent data indicate that DNA methylation of the eNOS promoter is present in EPCs, mesangioblasts and CD34-positive bone marrow cells (see Example 2). Conversely, the eNOS promoter is unmethylated in human umbilical vein endothelial cells (HUVEC) and microvascular endothelial cells (ECs), suggesting that eNOS transcription is epigenetically regulated and DNA methylation may be critical for the differentiation of human PCs into functionally competent ECs (See Example 2 and FIG. 11). The accumulation of methylated CpG in the eNOS promoter opposes the binding of the transcription factors Sp1, Sp3 and Ets1 to their consensus sequences interfering with gene expression (130). In fact, the inhibition of DNA methyltransferases by 5-azacytidine induces the upregulation of eNOS mRNA in ECs and non-EC types (130). These observations support the notion that DNA methylation may be operative in the regulation of VPC, MPC and BMPC growth and lineage commitment.

In one embodiment, the present invention provides a method of enhancing progenitor cell differentiation comprising exposing human adult progenitor cells to one or more inhibitors of DNA methyltransferases, wherein said progenitor cells exhibit enhanced differentiation as compared to progenitor cells not exposed to the one or more inhibitors of DNA methyltransferases. The human adult progenitor cells may be VPCs, MPCs, or BMPCs. In some embodiments, inhibition of DNA methyltransferases causes the human adult progenitor cells to differentiate into endothelial cells. Expression of genes of the endothelial cell lineage, such as eNOS and E-cadherin, may be upregulated following inhibition of DNA methyltransferases. In other embodiments, inhibition of DNA methyltransferases causes the human adult progenitor cells to differentiate into smooth muscle cells. Expression of genes of the smooth muscle cell lineage, such as SRF and GATA6, may be upregulated following inhibition of DNA methyltransferases. In still other embodiments, inhibition of DNA methyltransferases causes the human adult progenitor cells to differentiate into cardiomyocytes. Expression of genes of the myocyte cell lineage, such as Nkx2.5 and MEF2, may be upregulated following inhibition of DNA methyltransferases. Suitable inhibitors of DNA methyltransferases include, but are not limited to, 2-pyrimidone-1-b-D-riboside, 5-azacytidine, adenosyl-ornithine, and 2-(1,3-Dioxo-1,3-dihydro-2H-isoindol-2-yl)-3-(1H-indol-3-yl)propionic acid.

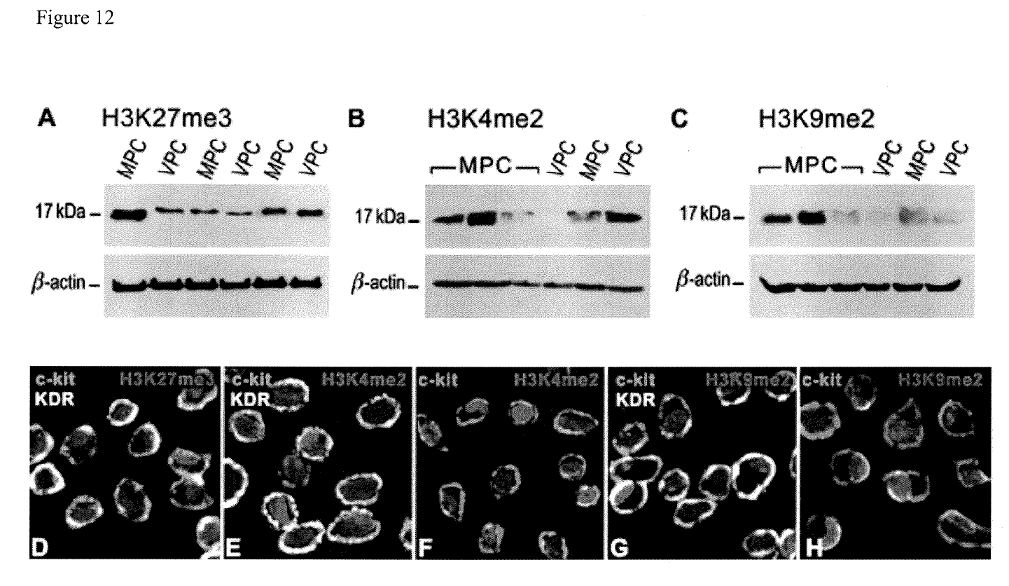

Histone acetylation is associated with increased transcription while histone methylation with upregulation or silencing of gene expression (112, 113, 116, 118). The differential effect of histone methylation is conditioned by the lysine residue involved and the degree of methylation: one, two or three methyl groups (131). The undifferentiated state of VPCs, MPCs and BMPCs may be conditioned by a bivalent chromatin configuration in which inactivating and activating marks coexist (132). These changes may result in repression of lineage-related genes and activation of stemness-related genes. This bivalent chromatin configuration is predicted to be lost with PC commitment. Epigenetic inactivation of multipotency-associated genes and activation of lineage-related genes may characterize cell differentiation. Results at the genome-wide level document that epigenetic mechanisms are present in MPCs and VPCs (see FIG. 12).

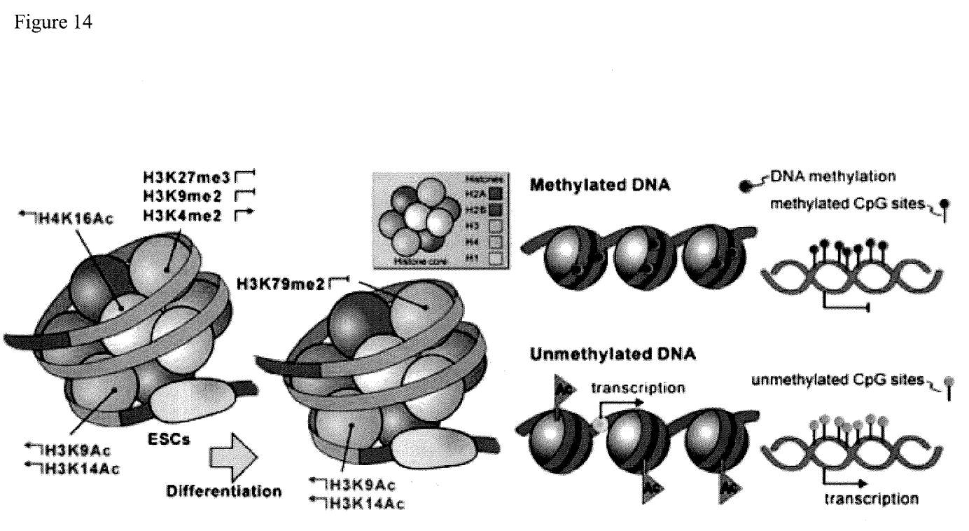

In undifferentiated PCs, the repression of lineage-related genes may be achieved by a bivalent chromatin structure of their promoter regions mimicking observations in ESCs (132). As shown schematically in FIG. 14, in ESCs this bivalent chromatin conformation is characterized by methylation of histone H3 at lysine 27 and lysine 4. Tri-methylation of histone H3 at lysine 27 (H3K27me3) negatively regulates transcription by promoting the generation of a compact chromatin structure (133, 134). Methylation of histone H3 at lysine 4 positively or, at times, negatively regulates transcription by recruiting nucleosome remodeling enzymes and histone acetylases (135-138). Di-methylation of histone H3 at lysine 4 (H3K4me2) and tri-methylation of histone H3 at lysine 4 (H3K4me3) are present in transcriptionally active chromatin regions (139). This bivalent chromatin conformation may represent a condition in which, following the removal of the repressive function of H3K27me3, lineage-related genes are in place for transcriptional activation by H3K4me2/3 (132). While H3K27me3 constitutes the major repressive mark in ESCs, in adult human MPCs and VPCs this function may be replaced by di-methylation of histone H3 at lysine 9 (H3K9me2) (140). Recent data indicate that undifferentiated VPCs and MPCs display a bivalent chromatin configuration characterized by the presence of H3K27me3 and H3K4me2 (see FIG. 12). However, in contrast to ESCs, H3K9me2 was the most pronounced repressive modification in human PCs. The level of H3K9me2 expression appears to be linked to the undifferentiated state of both VPCs and MPCs (FIG. 12). H3K27me3, H3K9me2 and H3K4me2 may be present in the promoters of the lineage-related genes Nkx2.5, MEF2, eNOS, E-cadherin, SRF and GATA6 and may be responsible for their repression in human undifferentiated VPCs, MPCs and BMPCs. Thus, differentiation of human progenitor cells may be induced by promoting demethylation of these specific lysine residues on histone 3.

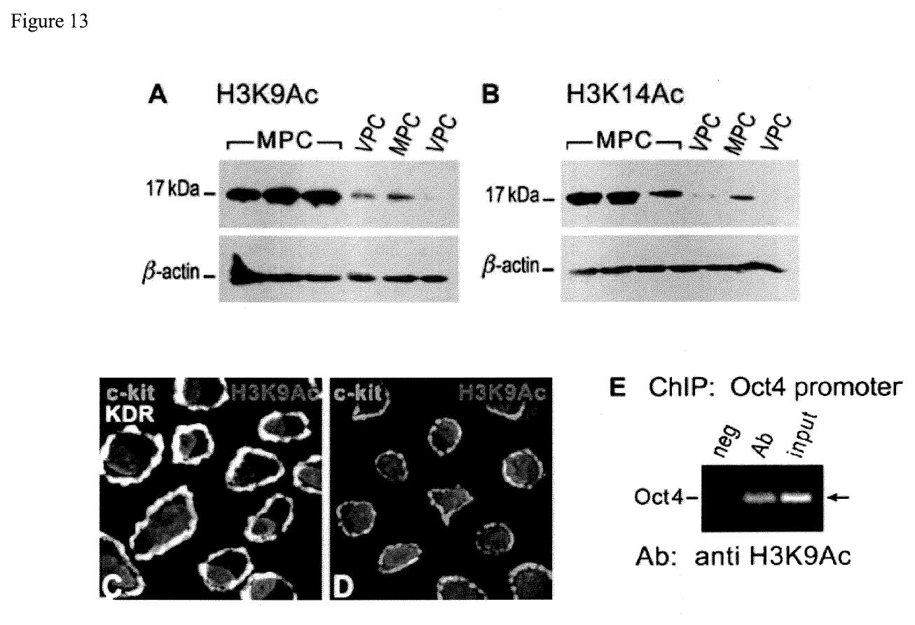

The activation of stemness-related genes may be mediated by global lysine acetylation in histone H3 and H4 (107, 112, 113). In the inner mass, undifferentiated cells show acetylation of histone H4 at lysine 16 (H4K16Ac) in the promoter of Oct4 and Nanog (117). H4K16Ac destabilizes the architecture of nucleosomes favoring the access of transcription factors and chromatin modifying enzymes to DNA (117). VPCs and MPCs exhibit two acetylation sites in histone H3 at lysine 9 (H3K9Ac) and lysine 14 (H3K14Ac). However, these genome-wide epigenetic modifications are more pronounced in MPCs than in VPCs (see FIG. 13). The promoter of Oct4 which regulates pluripotency and self-renewal of ESCs is selectively enriched in acetylated H3 at lysine 9. H3K9Ac and H3K14Ac may target promoter regions of Oct4 and Nanog in VPCs, MPCs and BMPCs.

The repression of stemness-related genes is critical for PC differentiation. Genes that encode Oct4 and Nanog may be silenced during PC commitment (140). This may be mediated by histone methylation and deacetylation. A similar epigenetic inactivation has to occur for lineage-related genes which are not implicated in the developmental choice of PCs (122). For example, the differentiation of a VPC into a SMC has to involve upregulation of SMC-related genes and repression of genes associated with the acquisition of the EC lineage. Bivalent chromatin domains typical of PCs may be replaced during differentiation by large regions of methylation at lysine 4, lysine 9 or lysine 27. These modified regions may provide epigenetic memory to maintain lineage-specific expression (141, 142). In addition to lysine methylation, loss of acetylation may result in inactivation of sternness genes.

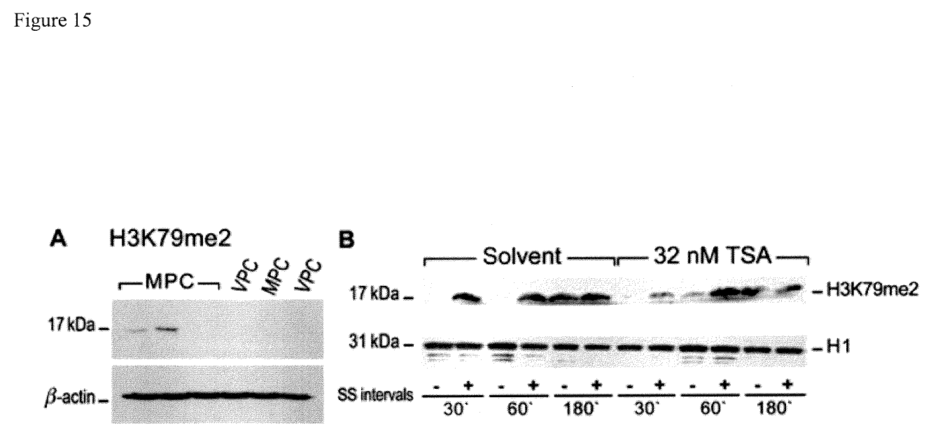

The activation of lineage-related genes has been documented in differentiating ESCs (119) and HSCs (143-145) in which tissue-specific chromatin domains are primed by epigenetic modifications, including acetylation of histone H3 at lysine 9 (H3K9Ac) and 14 (H3K14Ac). Additionally, di-methylation of histone H3 at lysine 79 (H3K79me2) occurs with stem cell differentiation and involves the globular domain of histone H3 (146). H3K9Ac and H3K14Ac are present in MPCs and VPCs while H3K79me2 is occasionally detected in MPCs (see FIG. 15). H3K79me2 has not been observed in VPCs.

The enzyme systems regulating DNA methylation, histone methylation and histone acetylation have largely been characterized (149-154). Histone deacetylases (HDACs) modulate vessel integrity, remodeling and growth (155, 156), which are critical variables of the failing heart (157, 158). Additionally, HDACs are implicated in the myocardial hypertrophic response (159-162) and the balance between myocyte formation and death (163). Importantly, HDAC isozymes have differential effects on the remodeling of the overloaded heart by enhancing or inhibiting myocyte growth (159, 162-164). Lysine acetylation of histones affects the conformation of chromatin, loosening the contacts between DNA and nucleosomes and, thereby, facilitating the decompaction of chromatin and its accessibility to transcription-promoting factors (108, 117, 118). Conversely, lysine deacetylation favors the methylation of lysine residues promoting the formation of heterochromatin and gene silencing or phosphorylation of adjacent serine residues (107, 109, 112). Non-histone targets of HDACs comprise the transcription factors p53, GATA4 and MEF2 and connexin 43 (165-167). Thus, inhibition of histone deacetylase activity promotes gene activation and transcription of particular genes.

The present invention provides a method for enhancing progenitor cell proliferation. In one embodiment, the method comprises exposing human adult progenitor cells to one or more HDAC inhibitors, wherein said progenitor cells exhibit enhanced proliferation as compared to progenitor cells not exposed to the one or more HDAC inhibitors. In another embodiment, the one or more HDAC inhibitors target a class I and/or class II HDAC enzyme. In another embodiment, the one or more HDAC inhibitors target class IIa HDACs (e.g. HDAC4, 5, 7, 9).

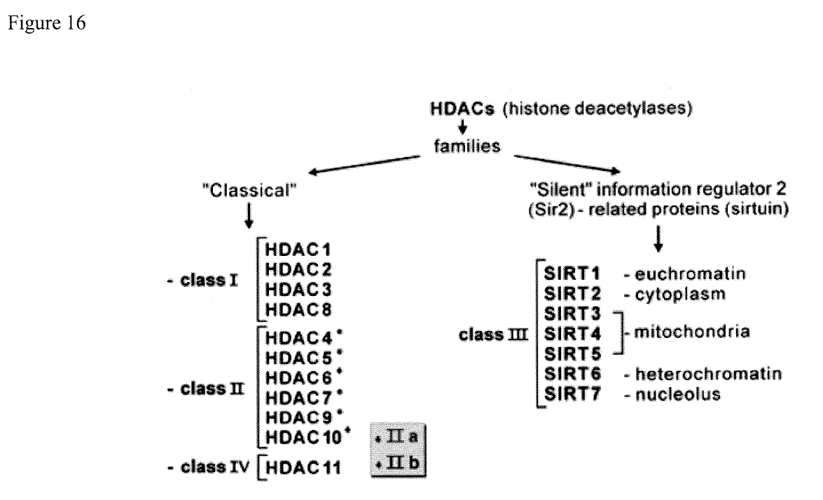

HDACs are divided in four classes (see FIG. 16). Class I HDACs possess sequence homology to members of classes II and IV but not to class III. Class I, II and IV HDACs are zinc-dependent enzymes while the deacetylase activity of class III HDACs is NAD+ dependent (154).

Class I HDACs correspond to HDAC1-3 and 8 which are ubiquitously expressed. HDAC1 and 2 are restricted to the nucleus (168) while HDAC3 can be detected in the nucleus, cytoplasm and plasma membrane (169). HDAC1, 2 and 3 are responsible for most of the deacetylase activity within the cell (169). In the embryonic heart, HDAC2 inhibits cardiomyogenesis (163). Deletion of HDAC2 leads to perinatal mortality with obliteration of the lumen of the right ventricle, excessive hyperplasia and cardiomyocyte apoptosis (163). HDAC2 deficiency prevents myocyte hypertrophy in the adult heart (162). HDAC3 deacetylates MEF2D repressing MEF2-dependent transcription and cardiomyogenesis (170). HDAC8 was thought to be located only in the nucleus (171) but it has also been found to be associated with SM actin in the cytoskeleton of SMCs where it may enhance cell contractility (172).

Class II HDACs include HDAC4-7, 9 and 10. Class II HDACs are further subdivided into class IIa (HDAC4, 5, 7, 9) and IIb (HDAC6, 10). Class IIa HDACs act as transcriptional co-repressors (173); they do not bind directly to DNA but are recruited to target promoter regions by sequence specific DNA binding proteins (173, 174). Class IIa HDACs repress a large number of transcriptional regulators involved in the differentiation program of a wide variety of cells (175). The canonical example of this function is the interaction between class IIa HDACs and MEF2 transcription factors (176-181). Class IIa HDACs have the property to undergo nuclear/cytoplasmic shuttling by phosphorylation/dephosphorylation (182); dephosphorylation leads to their nuclear accumulation and gene silencing while phosphorylation results in cytoplasmic sequestration and gene expression (183-185).

Class IIb HDACs comprise HDAC6 and 10. In the nucleus, HDAC6 functions as a transcriptional co-repressor (186) and in the cytoplasm regulates aggresome formation (187). HDAC10 is widely expressed, localizes to the nucleus and cytoplasm and attenuates weakly transcriptional activity (186)

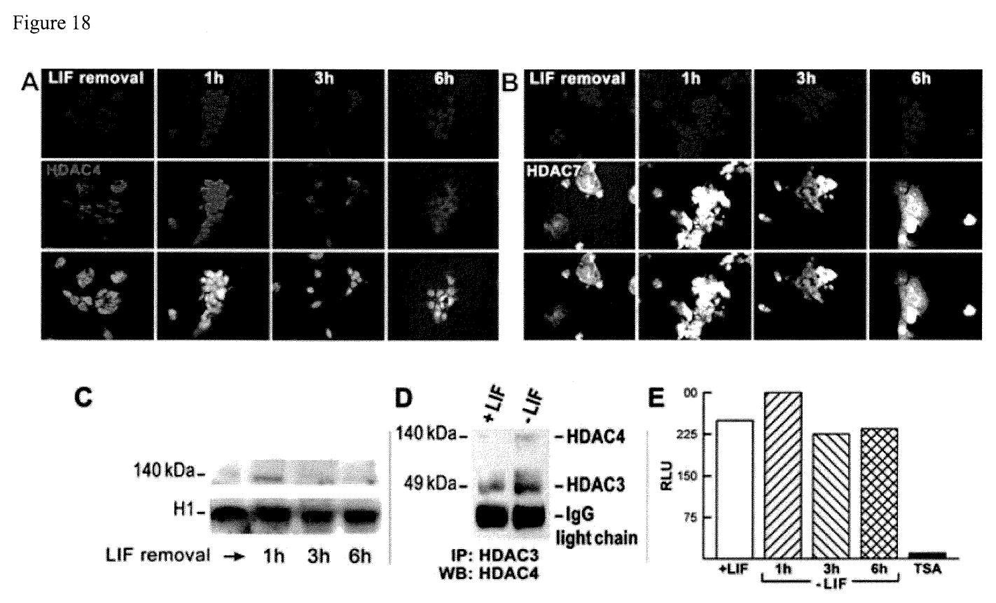

Class III HDACs correspond to sirtuins (SIRT), a largely conserved family of proteins, which in mammals consists of 7 members (188, 189). SIRT1-7 have different cellular localizations (see FIG. 18). SIRT1-3 and SIRT5 possess deacetylase activity (190-193). SIRT1 promotes the formation of compact heterochromatin and gene silencing by deacetylating lysine residues at position 9 and 26 of histone H1, position 14 of histone H3 and position 16 of histone H4 (194, 195). SIRT1 exerts multiple cellular functions by interacting with non-histone targets. SIRT1 negatively regulates the activity of HAT-p300 (196) and mediates p53 deacetylation suppressing apoptosis (191, 197). Importantly, SIRT1 represses myogenesis by deacetylating lysine 424 of MEF2 (198).

Class IV HDACs comprise HDAC11 which has features of class I and II HDACs. HDAC11 is restricted to the brain, heart, skeletal muscle, kidney and testis suggesting that its function may be tissue-specific. HDAC11 resides in the nucleus and forms a protein complex with HDAC6 (199).

In another embodiment, the present invention provides a method of enhancing progenitor cell differentiation comprising exposing human adult progenitor cells to one or more HDAC inhibitors, wherein said progenitor cells exhibit enhanced differentiation as compared to progenitor cells not exposed to the one or more HDAC inhibitors. In one embodiment, the one or more HDAC inhibitors target a class I and/or class II HDAC enzyme. In another embodiment, the one or more HDAC inhibitors target class IIa HDACs (e.g. HDAC4, 5, 7, 9).

The present invention also provides a method of restoring progenitor cell function to aged adult progenitor cells. In one embodiment, the method comprises exposing said aged progenitor cells to one or more HDAC inhibitors, wherein said progenitor cells exhibit increased expression of at least one stem cell related gene as compared to aged progenitor cells not exposed to the one or more HDAC inhibitors. The at least one stem related gene may be Oct4 or Nanog. In another embodiment, the aged progenitor cells are isolated from a subject suffering from heart failure.

“Restoring progenitor cell function” refers to the ability of progenitor cells to renew themselves through mitosis as well as differentiate into various specialized cell types without giving rise to senescent daughter cells (i.e. cells that express senescent markers such as p16INK4a). Thus, treatment of aged progenitor cells with one or more HDAC inhibitors preferably improves the ability of the treated progenitor cells to generate non-senescent cells as compared to untreated aged progenitor cells. Alternatively or additionally, stimulation of the enzymatic activity of histone acetyltransferases (HATs) in the aged progenitor cells may be used to restore progenitor cell function.

In another embodiment, the method of restoring progenitor cell function to aged adult progenitor cells comprises increasing SIRT1 activity in the aged progenitor cells. SIRT1, a class III HDAC, is downregulated with aging (261) and in senescent cells (262). Non-histone targets of SIRT1 include p53 and FOXO. SIRT1 deacetylates p53 decreasing its function (265). Increased p53 acetylation is associated with senescence while the increased activity of SIRT1 extends replicative lifespan of human smooth muscle cells. Thus, high level of SIRT1 expression and activity characterize young cells leading to deacetylation of p53, p53 degradation, and cell proliferation together with deacetylation of histones and selective gene silencing (266). In some embodiments, SIRT1 activity may be increased in aged progenitor cells by transfecting the progenitor cells with an expression plasmid encoding SIRT1.

Histone deacetylase inhibitors that are suitable for use in the methods of the invention include proteins, peptides, DNA molecules (including antisense), inhibitory RNA molecules as well as small molecules. Some non-limiting examples of histone deacetylase inhibitors include, but are not limited to, MS27-275, AN-9, apicidin derivatives, Baceca, CBHA, CHAPs, chlamydocin, CS-00028, CS-055, EHT-0205, FK-228, FR-135313, G2M-777, HDAC-42, LBH-589, MGCD-0103, NSC-3852, PXD-101, pyroxamide, SAHA derivatives, suberanilohydroxamic acid, tacedinaline, VX-563, MC1568, trichostatin A, and zebularine. In one embodiment, the one or more HDAC inhibitor is selected from the group consisting of trichostatin A, MS27-275, and MC1-568. In some embodiments, the one or more HDAC inhibitor targets a class I or class II HDAC enzyme, such HDACs 1, 2, 3, 4, 5, 6, 7, 8, 9, and 10. In other embodiments, the one or more HDAC inhibitor targets the class IIa HDAC enzymes, such as HDACs 4, 5, 7, and 9. In still other embodiments, more than one HDAC inhibitor can be employed, wherein one inhibitor targets a class I HDAC enzyme and a second inhibitor targets a class II or class IIa HDAC enzyme. Novel inhibitors that may be developed for any member of the class I or class II HDAC enzymes is also contemplated for use in the methods of the invention.

In some embodiments, HDAC inhibitors are antisense oligonucleotides or inhibitory RNA molecules, such as small interfering RNAs (siRNAs) or small hairpin RNAs (shRNAs). Antisense oligonucleotides, siRNA molecules, or shRNA molecules can be designed to target any of the class I or class II HDAC enzymes. In a preferred embodiment, the HDAC inhibitor is a siRNA molecule targeted to HDAC4, HDAC5, HDAC7, and HDAC 9. One of skill in the art is able to determine the sequences of the particular HDAC enzyme to be targeted and design appropriate antisense oligonucleotides, siRNAs, or shRNAs without undue experimentation.

The antisense oligonucleotides may be ribonucleotides or deoxyribonucleotides. Preferably, the antisense oligonucleotides have at least one chemical modification. Antisense oligonucleotides may be comprised of one or more “locked nucleic acids”. “Locked nucleic acids” (LNAs) are modified ribonucleotides that contain an extra bridge between the 2′ and 4′ carbons of the ribose sugar moiety resulting in a “locked” conformation that confers enhanced thermal stability to oligonucleotides containing the LNAs. Alternatively, the antisense oligonucleotides may comprise peptide nucleic acids (PNAs), which contain a peptide-based backbone rather than a sugar-phosphate backbone. Other chemical modifications that the antisense oligonucleotides may contain include, but are not limited to, sugar modifications, such as 2′-O-alkyl (e.g. 2′-O-methyl, 2′-O-methoxyethyl), 2′-fluoro, and 4′ thio modifications, and backbone modifications, such as one or more phosphorothioate, morpholino, or phosphonocarboxylate linkages. In some embodiments, suitable antisense oligonucleotides are 2′-O-methoxyethyl “gapmers” which contain 2′-O-methoxyethyl-modified ribonucleotides on both 5′ and 3′ ends with at least ten deoxyribonucleotides in the center. These “gapmers” are capable of triggering RNase H-dependent degradation mechanisms of RNA targets. Other modifications of antisense oligonucleotides to enhance stability and improve efficacy, such as those described in U.S. Pat. No. 6,838,283, which is herein incorporated by reference in its entirety, are known in the art and are suitable for use in the methods of the invention. Preferable antisense oligonucleotides useful for inhibiting the activity of a particular HDAC enzyme comprise a sequence that is at least partially complementary to the particular HDAC nucleotide sequence, e.g. at least about 75%, 80%, 85%, 90%, 95%, 96%, 97%, 98%, or 99% complementary to the particular HDAC nucleotide sequence. In one embodiment, the antisense oligonucleotide comprises a sequence that is 100% complementary to the particular HDAC nucleotide sequence.

The inhibitory RNA molecule (e.g. siRNA or shRNA) may have a double stranded region that is at least partially identical and partially complementary to a particular HDAC nucleotide sequence. The double-stranded regions of the inhibitory RNA molecule may comprise a sequence that is at least partially identical and partially complementary, e.g. about 75%, 80%, 85%, 90%, 95%, 96%, 97%, 98%, or 99% identical and complementary, to the particular HDAC nucleotide sequence. In one embodiment, the double-stranded regions of the inhibitory RNA molecule may contain 100% identity and complementarity to the particular HDAC nucleotide sequence.

The antisense oligonucleotides or inhibitory RNA molecules may be introduced into progenitor cells, e.g. aged progenitor cells, by direct transfection using standard methods in the art. Such methods include, but are not limited to, lipofection, DEAE-dextran-mediated transfection, microinjection, protoplast fusion, calcium phosphate precipitation, electroporation, and biolistic transformation. Alternatively, the antisense oligonucleotides or inhibitory RNA molecules may be expressed in the progenitor cells from a vector. A “vector” is a composition of matter which can be used to deliver a nucleic acid of interest to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. Examples of viral vectors include, but are not limited to, adenoviral vectors, adeno-associated virus vectors, retroviral vectors, lentiviral vectors and the like. An expression construct can be replicated in a living cell, or it can be made synthetically. For purposes of this application, the terms “expression construct,” “expression vector,” and “vector,” are used interchangeably to demonstrate the application of the invention in a general, illustrative sense, and are not intended to limit the invention.

In one embodiment, a vector for expressing the antisense oligonucleotide or inhibitory RNA molecule targeted to a particular HDAC enzyme comprises a promoter “operably linked” to the nucleic acid molecule. The phrase “operably linked” or “under transcriptional control” as used herein means that the promoter is in the correct location and orientation in relation to a polynucleotide to control the initiation of transcription by RNA polymerase and expression of the polynucleotide. Several promoters are suitable for use in the vectors for expressing the antisense oligonucleotide or inhibitory RNA molecule, including, but not limited to, RNA pol I promoter, RNA pol II promoter, RNA pol III promoter, and cytomegalovirus (CMV) promoter. Other useful promoters are discernible to one of ordinary skill in the art. In some embodiments, the promoter is an inducible promoter that allows one to control when the antisense oligonucleotide or inhibitory RNA molecule is expressed. Suitable examples of inducible promoters include tetracycline-regulated promoters (tet on or tet off) and steroid-regulated promoters derived from glucocorticoid or estrogen receptors. Alternatively, the promoter operably linked to the antisense oligonucleotide or inhibitory RNA molecule may be a promoter of a stem related gene, such as Oct4 or Nanog.

Preferably, the progenitor cells used in the methods of the invention are lineage negative, c-kit positive adult progenitor cells. The adult progenitor cells may be adult vascular progenitor cells (VPCs), adult myocyte progenitor cells (MPCs), adult bone marrow progenitor cells (BMPCs), or combinations thereof. VPCs are lineage negative, c-kit positive, and KDR (e.g. flk1) positive, and differentiate predominantly into endothelial cells and smooth muscle cells. MPCs are lineage negative, c-kit positive, and KDR (e.g. flk1) negative, and differentiate predominantly into cardiomyocytes. BMPCs are c-kit positive and lineage negative, and differentiate into endothelial cells, smooth muscle cells, and cardiomyocytes. In some embodiments, the adult progenitor cells are human progenitor cells, that is human vascular progenitor cells, human myocyte progenitor cells, and human bone marrow progenitor cells.

Progenitor cells may be isolated from tissue specimens, such as myocardium or bone marrow, obtained from a subject or patient, for instance an aging patient or a patient suffering from heart failure. By way of example, myocardial tissue specimens obtained from the subject's heart may be minced and placed in appropriate culture medium. Cardiac progenitor cells growing out from the tissue specimens can be observed in approximately 1-2 weeks after initial culture. At approximately 4 weeks after the initial culture, the expanded progenitor cells may be collected by centrifugation. An exemplary method for obtaining bone marrow progenitor cells from a subject is described as follows. Bone marrow may be harvested from the iliac crests using a needle and the red blood cells in the sample may be lysed using standard reagents. Bone marrow progenitor cells are collected from the sample by density gradient centrifugation. Optionally, the bone marrow progenitor cells may be expanded in culture. Other methods of isolating adult progenitor cells, such as bone marrow progenitor cells and cardiac progenitor cells (e.g. VPCs and MPCs), from a subject are known in the art and can be employed to obtain suitable progenitor cells for use in the methods of the invention. U.S. Patent Application Publication No. 2006/0239983, filed Feb. 16, 2006, which is herein incorporated by reference in its entirety, describes media appropriate for culturing and expanding adult progenitor cells. However, one of ordinary skill in the art would be able to determine the necessary components and modify commonly used cell culture media to be employed in culturing the isolated progenitor cells of the invention.

It is preferable that the progenitor cells of the invention are lineage negative. Lineage negative progenitor cells can be isolated by various means, including but not limited to, removing lineage positive cells by contacting the progenitor cell population with antibodies against lineage markers and subsequently isolating the antibody-bound cells by using an anti-immunoglobulin antibody conjugated to magnetic beads and a biomagnet. Alternatively, the antibody-bound lineage positive stem cells may be retained on a column containing beads conjugated to anti-immunoglobulin antibodies. For instance, lineage negative bone marrow progenitor cells may be obtained by incubating mononuclear cells isolated from a bone marrow specimen with immunomagnetic beads conjugated with monoclonal antibodies for CD3 (T lymphocytes), CD20 (B lymphocytes), CD33 (myeloid progenitors), CD14 and CD15 (monocytes). The cells not bound to the immunomagnetic beads represent the lineage negative bone marrow progenitor cell fraction and may be isolated. Similarly, cells expressing markers of the cardiac lineage (e.g. markers of vascular cell or cardiomyocyte commitment) may be removed from cardiac progenitor cell populations to isolate lineage negative cardiac progenitor cells. Markers of the vascular lineage include, but are not limited to, GATA6 (SMC transcription factor), Ets1 (EC transcription factor), Tie-2 (angiopoietin receptors), VE-cadherin (cell adhesion molecule), CD62E/E-selectin (cell adhesion molecule), alpha-SM-actin (α-SMA, contractile protein), CD31 (PECAM-1), vWF (carrier of factor VIII), Bandeiraera simplicifolia and Ulex europaeus lectins (EC surface glycoprotein-binding molecules). Markers of the myocyte lineage include, but are not limited to, GATA4 (cardiac transcription factor), Nkx2.5 and MEF2C (myocyte transcription factors), and alpha-sarcomeric actin (α-SA, contractile protein).

In a preferred embodiment of the invention, the lineage negative progenitor cells express the stem cell surface marker, c-kit, which is the receptor for stem cell factor. Positive selection methods for isolating a population of lineage negative progenitor cells expressing c-kit are well known to the skilled artisan. Examples of possible methods include, but are not limited to, various types of cell sorting, such as fluorescence activated cell sorting (FACS) and magnetic cell sorting as well as modified forms of affinity chromatography. In a preferred embodiment, the lineage negative progenitor cells are c-kit positive.

Vascular progenitor cells are isolated by selecting cells expressing the VEGFR2 receptor, KDR (e.g. flk1), from the c-kit positive progenitor cell population, isolated as described above. Thus, vascular progenitor cells are lineage negative, c-kit positive, and KDR positive. Similarly, myocyte progenitor cells are isolated from the c-kit progenitor cell population by selecting cells that do no express KDR. Therefore, myocyte progenitor cells are lineage negative, c-kit positive, and KDR negative. Similar methods for isolating c-kit positive progenitor cells may be employed to select cells that express or do not express the KDR receptor (e.g. immunobeads, cell sorting, affinity chromatography, etc.).

Isolated lineage negative, c-kit positive progenitor cells (e.g. VPCs, BMPCs, and MPCs) may be plated individually in single wells of a cell culture plate and expanded to obtain clones from individual progenitor cells. In some embodiments, cardiac progenitor cells that are c-kit positive and KDR positive are plated individually to obtain pure cultures of vascular progenitor cells. In other embodiments, cardiac progenitor cells that are c-kit positive and KDR negative are plated individually to obtain pure cultures of myocyte progenitor cells.

The isolated progenitor cell populations, e.g. VPCs, BMPCs, and MPCs, can be treated with one or more HDAC inhibitors as described herein. In some embodiments, the progenitor cells may express an HDAC inhibitor, such as an antisense oligonucleotide or inhibitory RNA molecule (e.g. siRNA or shRNA) directed to a specific HDAC enzyme.

The present invention also provides a method of treating heart failure in a subject in need thereof. In one embodiment, the method comprises isolating adult progenitor cells from a tissue specimen from the subject; exposing said isolated progenitor cells to one or more HDAC inhibitors; and administering said treated progenitor cells to the subject's heart, wherein said progenitor cells generate new coronary vessels and myocardium, thereby improving cardiac function. Increased cardiac function may be reflected as increased exercise capacity, increased cardiac ejection volume, decreased left ventricular end diastolic pressure, decreased pulmonary capillary wedge pressure, increased cardiac output, increased cardiac index, lowered pulmonary artery pressures, decreased left ventricular end systolic and diastolic dimensions, decreased left and right ventricular wall stress, and decreased wall tension. The adult progenitor cells may be human vascular progenitor cells, human myocyte progenitor cells, human bone marrow progenitor cells, or combinations thereof. The progenitor cells may be treated with any of the HDAC inhibitors described herein. In a preferred embodiment, the one or more HDAC inhibitors target a class I and/or class II HDAC enzyme.

Preferably, at least one symptom of heart failure is reduced in the subject following administration of the treated progenitor cells. Symptoms of heart failure include, but are not limited to, fatigue, weakness, rapid or irregular heartbeat, dyspnea, persistent cough or wheezing, edema in the legs and feet, and swelling of the abdomen. The treated progenitor cells differentiate into cardiomyocytes, smooth muscle cells, and endothelial cells following their administration and assemble into myocardium and myocardial vessels (e.g. coronary arteries, arterioles, and capillaries) thereby restoring structure and function to the decompensated heart.

The present invention also includes a method of restoring structural and functional integrity to damaged myocardium in a subject in need thereof comprising isolating adult progenitor cells from a tissue specimen from the subject; exposing said isolated progenitor cells to one or more HDAC inhibitors; and administering said treated progenitor cells to the subject's heart, wherein said progenitor cells generate new coronary vessels and myocardium, thereby improving cardiac function. In some embodiments, the subject is suffering from a myocardial infarction and the damaged myocardium is an infarct. The adult progenitor cells may be vascular progenitor cells, myocyte progenitor cells, bone marrow progenitor cells, or combinations thereof.

In certain embodiments of the invention, the cardiac progenitor cells or bone marrow progenitor cells are activated in addition to being treated with an HDAC inhibitor prior to administration. Activation of the progenitor cells may be accomplished by exposing the progenitor cells to one or more cytokines. Suitable concentrations of the one or more cytokines for activating the progenitor cells include a concentration of about 0.1 to about 500 ng/ml, about 10 to about 500 ng/ml, about 20 to about 400 ng/ml, about 30 to about 300 ng/ml, about 50 to about 200 ng/ml, or about 80 to about 150 ng/ml. In one embodiment, the concentration of one or more cytokines is about 25, about 50, about 75, about 100, about 125, about 150, about 175, about 200, about 225, about 250, about 275, about 300, about 325, about 350, about 375, about 400, about 425, about 450, about 475, or about 500 ng/ml. In some embodiments, the cardiac progenitor cells or bone marrow progenitor cells are activated by contact with hepatocyte growth factor (HGF), insulin-like growth factor-1 (IGF-1), or variant thereof.

HGF positively influences stem cell migration and homing through the activation of the c-Met receptor (Kollet et al. (2003) J. Clin. Invest. 112: 160-169; Linke et al. (2005) Proc. Natl. Acad. Sci. USA 102: 8966-8971; Rosu-Myles et al. (2005) J. Cell. Sci. 118: 4343-4352; Urbanek et al. (2005) Circ. Res. 97: 663-673). Similarly, IGF-1 and its corresponding receptor (IGF-1R) induce cardiac stem cell division, upregulate telomerase activity, hinder replicative senescence and preserve the pool of functionally-competent cardiac stem cells in the heart (Kajstura et al. (2001) Diabetes 50: 1414-1424; Torella et al. (2004) Circ. Res. 94: 514-524; Davis et al. (2006) Proc. Natl. Acad. Sci. USA 103: 8155-8160). In some embodiments, the cardiac progenitor cells or bone marrow progenitor cells are contacted with HGF and IGF-1.

Some other non-limiting examples of cytokines that are suitable for the activation of the cardiac progenitor cells or bone marrow progenitor cells include Activin A, Bone Morphogenic Protein 2, Bone Morphogenic Protein 4, Bone Morphogenic Protein 6, Cardiotrophin-1, Fibroblast Growth Factor 1, Fibroblast Growth Factor 4, Flt3 Ligand, Glial-Derived Neurotrophic Factor, Heparin, Insulin-like Growth Factor-II, Insulin-Like Growth Factor Binding Protein-3, Insulin-Like Growth Factor Binding Protein-5, Interleukin-3, Interleukin-6, Interleukin-8, Leukemia Inhibitory Factor, Midkine, Platelet-Derived Growth Factor AA, Platelet-Derived Growth Factor BB, Progesterone, Putrescine, Stem Cell Factor, Stromal-Derived Factor-1, Thrombopoietin, Transforming Growth Factor-α, Transforming Growth Factor-β1, Transforming Growth Factor-β2, Transforming Growth Factor-β3, Vascular Endothelial Growth Factor, Wnt1, Wnt3a, and Wnt5a, as described in Kanemura et al. (2005) Cell Transplant. 14:673-682; Kaplan et al. (2005) Nature 438:750-751; Xu et al. (2005) Methods Mol. Med. 121:189-202; Quinn et al. (2005) Methods Mol. Med. 121:125-148; Almeida et al. (2005) J Biol. Chem. 280:41342-41351; Barnabe-Heider et al. (2005) Neuron 48:253-265; Madlambayan et al. (2005) Exp Hematol 33:1229-1239; Kamanga-Sollo et al. (2005) Exp Cell Res 311:167-176; Heese et al. (2005) Neuro-oncol. 7:476-484; He et al. (2005) Am J Physiol. 289:H968-H972; Beattie et al. (2005) Stem Cells 23:489-495; Sekiya et al. (2005) Cell Tissue Res 320:269-276; Weidt (2004) Stem Cells 22:890-896; Encabo et al (2004) Stem Cells 22:725-740; and Buytaeri-Hoefen et al. (2004) Stem Cells 22:669-674, the entire text of each of which is incorporated herein by reference.

Functional variants of the above-mentioned cytokines can also be employed in the invention. Functional cytokine variants would retain the ability to bind and activate their corresponding receptors. Variants can include amino acid substitutions, insertions, deletions, alternative splice variants, or fragments of the native protein. For example, NK1 and NK2 are natural splice variants of HGF, which are able to bind to the c-MET receptor. These types of naturally occurring splice variants as well as engineered variants of the cytokine proteins that retain function can be employed to activate the progenitor cells of the invention.

The present invention involves administering a therapeutically effective dose or amount of progenitor cells treated with one or more HDAC inhibitors to a subject's heart. An effective dose is an amount sufficient to effect a beneficial or desired clinical result. Said dose could be administered in one or more administrations. In some embodiments, at least three effective doses are administered to the subject's heart. In other embodiments, at least five effective doses are administered to the subject's heart. Each administration of progenitor cells may comprise a single type of progenitor cell (e.g. BMPC, VPC, or MPC) or may contain mixtures of the different types of progenitor cells. In one embodiment, bone marrow progenitor cells (BMPCs) are initially administered to the subject, and vascular progenitor cells (VPCs) and/or myocyte progenitor cells (MPCs) are administered at set intervals after the administration of BMPCs. Examples of suitable intervals include, but are not limited to, 1 week, 2 weeks, 3 weeks, 1 month, 2 months, 3 months, 6 months, 12 months, 18 months or 24 months.

An effective dose of progenitor cells may be from about 2×10⁴ to about 1×10⁷, more preferably about 1×10⁵ to about 6×10⁶, or most preferably about 2×10⁶. However, the precise determination of what would be considered an effective dose may be based on factors individual to each patient, including their size, age, extent of decompensation, amount of damaged myocardium, and type of repopulating progenitor cells (e.g. VPCs, MPCs, or BMPCs). One skilled in the art, specifically a physician or cardiologist, would be able to determine the number of progenitor cells that would constitute an effective dose without undue experimentation.

The HDAC inhibitor-treated progenitor cells may be administered to the heart by injection. The injection is preferably intramyocardial. As one skilled in the art would be aware, this is the preferred method of delivery for progenitor cells as the heart is a functioning muscle. Injection by this route ensures that the injected material will not be lost due to the contracting movements of the heart.

In another embodiment, the progenitor cells are administered by injection transendocardially or trans-epicardially. In another embodiment of the invention, the progenitor cells are administered using a catheter-based approach to deliver the trans-endocardial injection. The use of a catheter precludes more invasive methods of delivery wherein the opening of the chest cavity would be necessitated. As one skilled in the art would appreciate, optimum time of recovery would be allowed by the more minimally invasive procedure. A catheter approach involves the use of such techniques as the NOGA catheter or similar systems. The NOGA catheter system facilitates guided administration by providing electromechanic mapping of the area of interest, as well as a retractable needle that can be used to deliver targeted injections or to bathe a targeted area with a therapeutic. Any of the embodiments of the present invention can be administered through the use of such a system to deliver injections or provide a therapeutic. One of skill in the art will recognize alternate systems that also provide the ability to provide targeted treatment through the integration of imaging and a catheter delivery system that can be used with the present invention. Information regarding the use of NOGA and similar systems can be found in, for example, Sherman (2003) Basic Appl. Myol. 13: 11-14; Patel et al. (2005) The Journal of Thoracic and Cardiovascular Surgery 130:1631-38; and Perrin et al. (2003) Circulation 107: 2294-2302; the text of each of which are incorporated herein in their entirety.

In still another embodiment, the progenitor cells that have been treated with an HDAC inhibitor may be administered to a subject's heart by an intracoronary route. This route obviates the need to open the chest cavity to deliver the cells directly to the heart. One of skill in the art will recognize other useful methods of delivery or implantation which can be utilized with the present invention, including those described in Dawn et al. (2005) Proc. Natl. Acad. Sci. USA 102, 3766-3771, the contents of which are incorporated herein in their entirety.

The present invention also comprehends methods for preparing compositions, such as pharmaceutical compositions, including one or more of the different type of progenitor cells described herein (e.g. BMPCs, VPC, and MPCs) and a histone deacetylase inhibitor, for instance, for use in treating or preventing heart failure. In one embodiment, the composition comprises human bone marrow progenitor cells and a histone deacetylase inhibitor, wherein said bone marrow progenitor cells are lineage negative and c-kit positive. In another embodiment, the composition comprises human vascular progenitor cells and a histone deacetylase inhibitor, wherein said vascular progenitor cells are lineage negative, c-kit positive and KDR positive. In another embodiment, the composition comprises human myocyte progenitor cells and a histone deacetylase inhibitor, wherein said myocyte progenitor cells are lineage negative, c-kit positive and KDR negative. In some embodiments, the composition comprises a combination of human vascular progenitor cells, human myocyte progenitor cells, human bone marrow progenitor cells and a histone deacetylase inhibitor. For instance, the composition may comprise VPCs, MPCs, and a histone deacetylase inhibitor; VPCs, BMPCs, and a histone deacetylase inhibitor; MPCs, BMPCs, and a histone deacetylase inhibitor; or VPCs, MPCs, BMPCs, and a histone deacetylase inhibitor. In further embodiments, any of the compositions described herein may further comprise a pharmaceutically acceptable carrier.

Any of the histone deacetylase (HDAC) inhibitors disclosed herein may be used in the compositions of the invention, including pharmaceutical compositions. In one embodiment, the HDAC inhibitor targets class I or class II HDAC enzymes. In another embodiment, the HDAC inhibitor is trichostatin A, MS27-275, or MC1568. In still another embodiment, the HDAC inhibitor is an inhibitory RNA molecule, such as a siRNA or shRNA, targeted to a class I or class II HDAC enzyme. In some embodiments, the inhibitory RNA molecule is targeted to a class IIa HDAC enzyme, including HDAC4, HDAC5, HDAC7, and HDAC 9. In other embodiments, the human progenitor cells in the composition express the inhibitory RNA molecule. More than one HDAC inhibitor may be included in the compositions. For example, an inhibitor of a class I HDAC enzyme may be combined with a class II HDAC inhibitor or an inhibitor of one class IIa HDAC enzyme may be combined with a second inhibitor of another class IIa HDAC enzyme (e.g. HDAC 4 inhibitor and HDAC 7 inhibitor).

In an additionally preferred aspect, the pharmaceutical compositions of the present invention are delivered to a subject's heart via injection. These routes for administration (delivery) include, but are not limited to, subcutaneous or parenteral including intravenous, intraarterial (e.g. intracoronary), intramuscular, intraperitoneal, intramyocardial, transendocardial, trans-epicardial, intranasal administration as well as intrathecal, and infusion techniques. Accordingly, the pharmaceutical composition is preferably in a form that is suitable for injection.

When administering a therapeutic of the present invention (e.g. HDAC inhibitor-treated progenitor cells) parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils. In some embodiments, the progenitor cells may be separated from the HDAC inhibitor following exposure to the inhibitor. In such embodiments, the treated progenitor cells may be resuspended in a pharmaceutically acceptable carrier prior to administration to a subject.

Proper fluidity of the compositions can be maintained, for example, by the use of a coating such as lecithin, by the maintenance of the required particle size in the case of dispersion, and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions.

Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the progenitor cells and other compounds used in combination with the progenitor cells, such as the HDAC inhibitors.

Sterile injectable solutions can be prepared by incorporating the compounds utilized in practicing the present invention in the required amount of the appropriate solvent with various amounts of the other ingredients, as desired.

The pharmaceutical compositions of the present invention, e.g., comprising a therapeutic dose of progenitor cells (e.g. BMPCs, VPC, and MPCs) and a HDAC inhibitor, can be administered to the patient in an injectable formulation containing any compatible carrier, such as various vehicles, adjuvants, additives, and diluents. Other therapeutic agents to be administered as a combination therapy with the HDAC inhibitor-treated progenitor cells can be administered parenterally to the patient in the form of slow-release subcutaneous implants or targeted delivery systems such as monoclonal antibodies, iontophoretic, polymer matrices, liposomes, and microspheres.

Examples of compositions comprising a therapeutic of the invention include liquid preparations for parenteral, subcutaneous, intradermal, intramuscular, intracoronarial, intramyocardial or intravenous administration (e.g., injectable administration), such as sterile suspensions or emulsions. Such compositions may be in admixture with a suitable carrier, diluent, or excipient such as sterile water, physiological saline, glucose or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

The compositions can be isotonic, i.e., they can have the same osmotic pressure as blood and lacrimal fluid. The desired isotonicity of the compositions of this invention may be accomplished using sodium chloride, or other pharmaceutically acceptable agents such as dextrose, boric acid, sodium tartrate, propylene glycol or other inorganic or organic solutes. Sodium chloride is preferred particularly for buffers containing sodium ions.

Viscosity of the compositions may be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the agent selected. The important point is to use an amount which will achieve the selected viscosity. Viscous compositions are normally prepared from solutions by the addition of such thickening agents.

A pharmaceutically acceptable preservative can be employed to increase the shelf-life of the compositions. Benzyl alcohol may be suitable, although a variety of preservatives including, for example, parabens, thimerosal, chlorobutanol, or benzalkonium chloride may also be employed. A suitable concentration of the preservative will be from 0.02% to 2% based on the total weight although there may be appreciable variation depending upon the agent selected.

Those skilled in the art will recognize that the components of the compositions should be selected to be chemically inert with respect to the active compound. This will present no problem to those skilled in chemical and pharmaceutical principles, or problems can be readily avoided by reference to standard texts or by simple experiments (not involving undue experimentation), from this disclosure and the documents cited herein.

The inventive compositions of this invention are prepared by mixing the ingredients following generally accepted procedures. For example, isolated progenitor cells and a HDAC inhibitor can be resuspended in an appropriate pharmaceutically acceptable carrier and the mixture adjusted to the final concentration and viscosity by the addition of water or thickening agent and possibly a buffer to control pH or an additional solute to control tonicity. Generally the pH may be from about 3 to 7.5. Compositions can be administered in dosages and by techniques well known to those skilled in the medical and veterinary arts taking into consideration such factors as the age, sex, weight, and condition of the particular patient, and the composition form used for administration (e.g., liquid). Dosages for humans or other mammals can be determined without undue experimentation by the skilled artisan, from this disclosure, the documents cited herein, and the knowledge in the art.

Suitable regimes for initial administration and further doses or for sequential administrations also are variable, may include an initial administration followed by subsequent administrations; but nonetheless, may be ascertained by the skilled artisan, from this disclosure, the documents cited herein, and the knowledge in the art.

This invention is further illustrated by the following additional examples that should not be construed as limiting. The contents of all references, patents and published patent applications cited throughout this application, as well as the Figures, are incorporated herein by reference in their entirety.

### EXAMPLES

### Example 1

### Origin of Human Cardiac VPCs and MPCs

There are two main objectives of the experiments discussed in this Example: (1) to determine whether human vascular progenitor cells (VPCs) and myocyte progenitor cells (MPCs) are resident populations of cardiac progenitor cells (PCs) or represent subsets of bone marrow progenitor cells (BMPCs) and (2) to determine whether human VPCs and MPCs are two distinct PC classes or constitute two interrelated compartments of the cardiac PC pool.

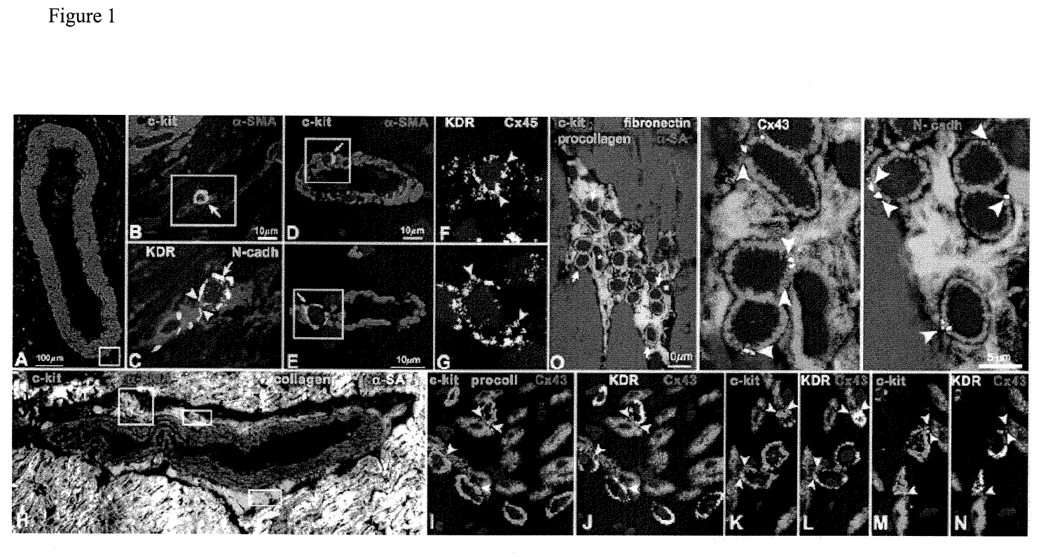

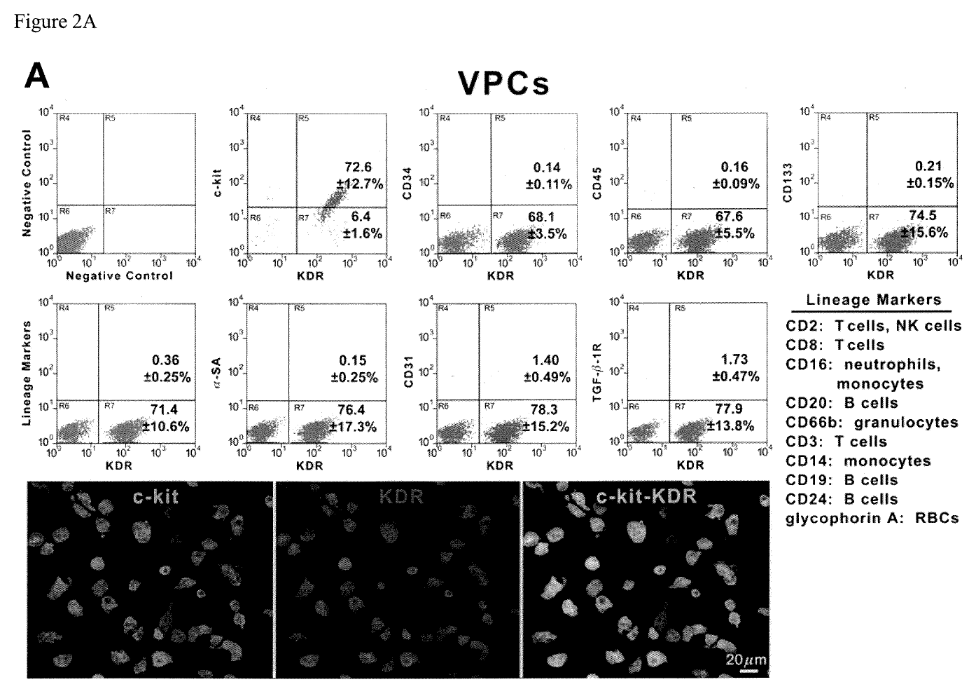

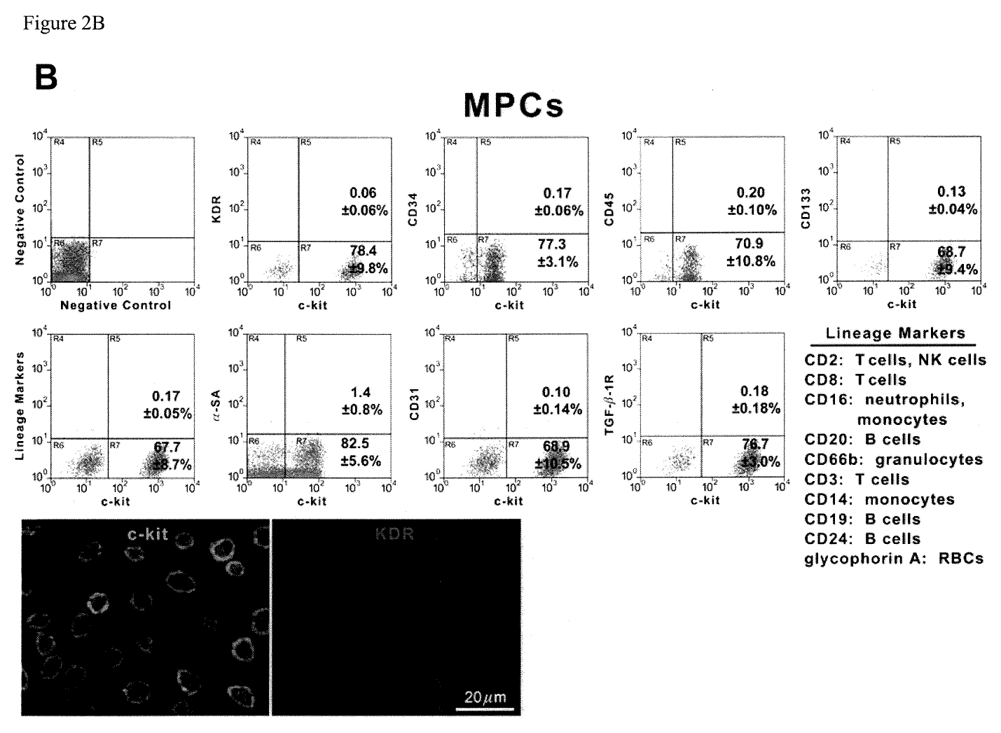

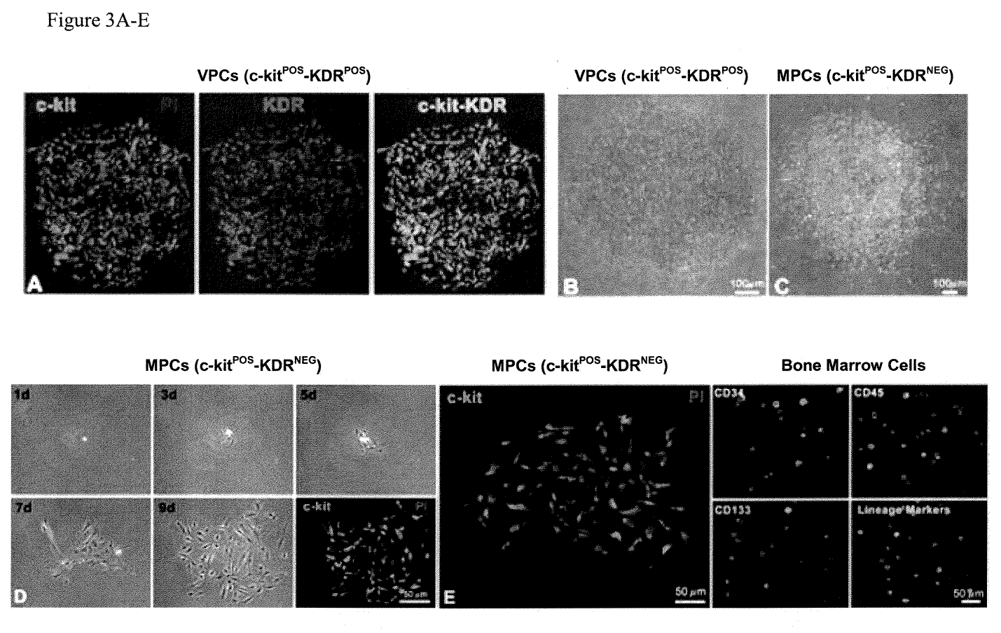

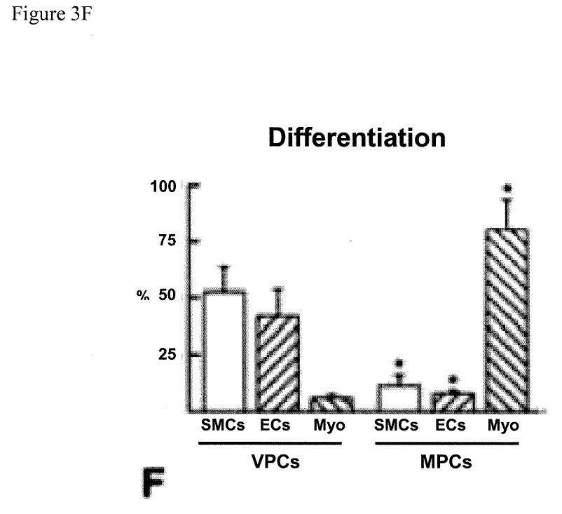

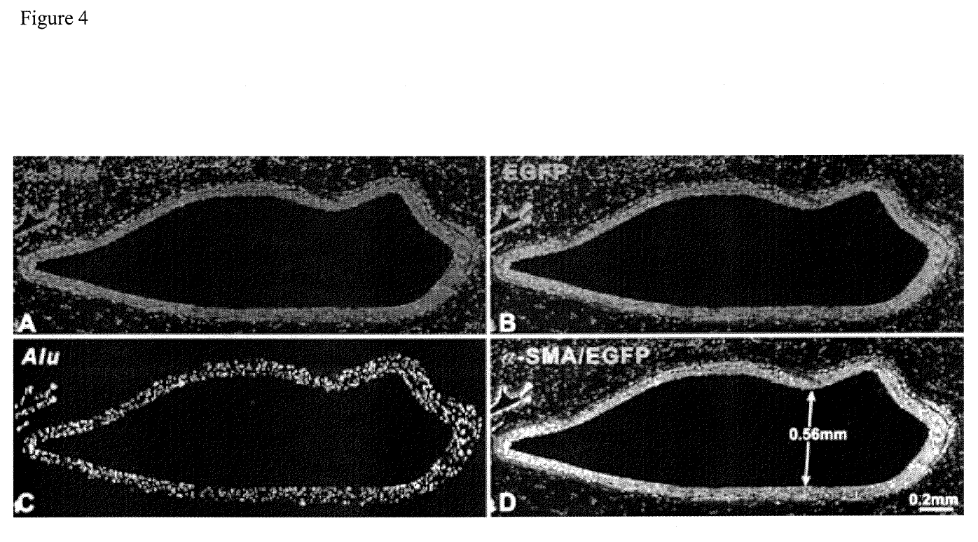

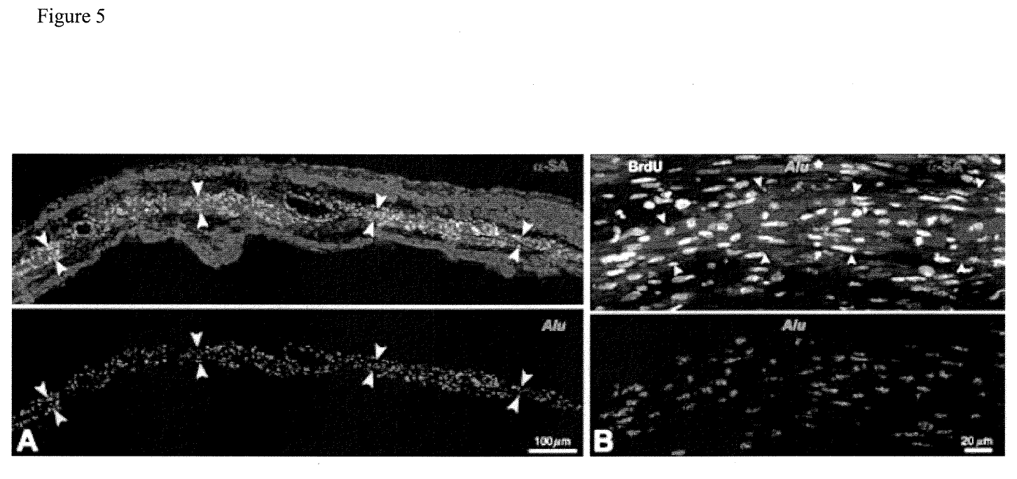

VPCs have been detected in the intima, media and adventitia of different classes of human coronary vessels suggesting that vascular niches are present in the coronary circulation and are distinct from myocardial niches in which MPCs are stored (FIG. 1). VPCs and MPCs have been isolated from the human heart and separately expanded in vitro (FIG. 2), and single cell clones have been obtained from individual human VPCs and MPCs (FIG. 3). Clonogenic human VPCs differentiate in vitro predominantly into vascular smooth muscle cells (SMCs) and endothelial cells (ECs), and clonogenic human MPCs differentiate in vitro predominantly into myocytes (FIG. 3). Transfer of human VPCs generate in vivo large conductive human coronary arteries, arterioles, and capillaries in immunosuppressed dogs with critical coronary artery stenosis or myocardial infarction (FIG. 4), and transfer of human MPCs generate in vivo a large number of cardiomyocytes in immunodeficient mice or immunosuppressed rats with myocardial infarction (FIG. 5). VPCs possess to a limited extent the ability to form cardiomyocytes and MPCs possess to a limited extent the ability to form coronary vessels (not shown).

Collectively, these findings document that VPCs and MPCs possess the fundamental properties of stem cells (1, 6, 11, 68-70); they are self-renewing, clonogenic and multipotent. VPCs and MPCs appear to be phenotypically and functionally distinct PC classes: VPCs possess specialized functions devoted to the turnover of ECs and SMCs and vasculogenesis while MPCs are responsible for myocyte homeostasis and cardiomyogenesis.

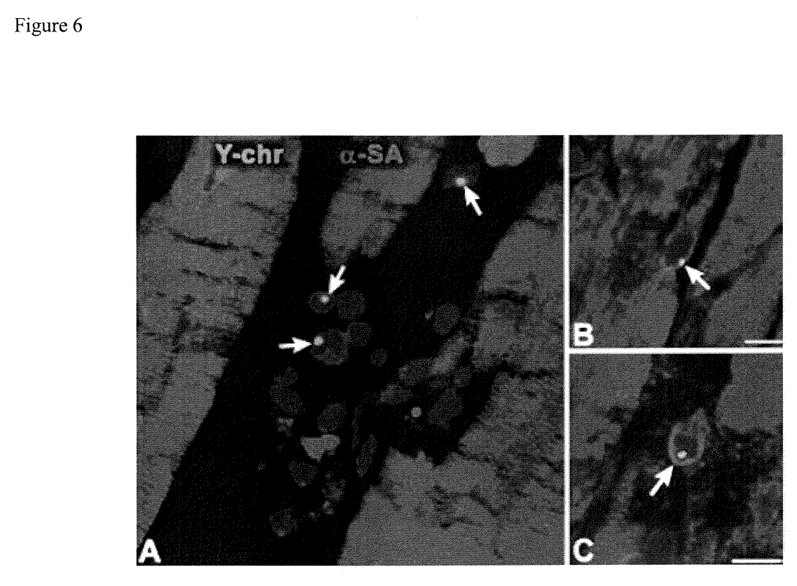

Since clonogenic VPCs and clonogenic MPCs are present in the human heart (FIGS. 1-5), the question is whether the two PC classes originate, live and die within the heart or the bone marrow continuously replenishes the heart with undifferentiated BMPCs that subsequently acquire cardiac characteristics. To address the question of whether growth regulation of coronary vessels and cardiomyocytes in humans is controlled by resident VPCs and MPCs which do not derive from the bone marrow, the hearts of patients who died following sex mismatched bone marrow transplantation are examined (FIG. 6). Cases in which female patients received bone marrow from male donors provide the opportunity to test whether male BMPCs and their progeny are present in the heart by the detection of Y-chromosome carrying cells within the myocardium (59, 75). Sex mismatched bone marrow transplantation mimics experimentally bone marrow transplantation with EGFP or β-gal positive cells and the formation of a chimeric blood and possibly heart. Moreover, the existence of cells of donor origin can be determined in the presence and absence of sex mismatched transplantation by PCR amplification of regions of the human genome with high polymorphic neutral sequence variation showing Mendelian inheritance as variable number of tandem repeats (VNTR) (200-204). The latter identifies at the DNA level molecular fingerprint of donor and recipient and does not require sex mismatch.

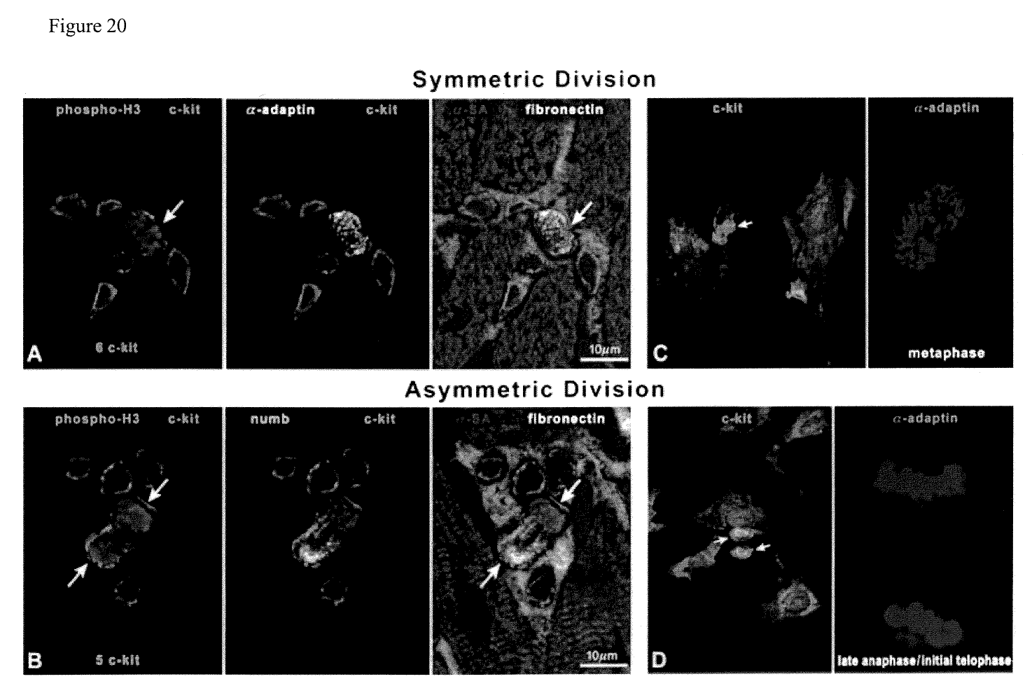

To understand the actual role of BMPCs in cardiac homeostasis in humans, the contribution of male BMPCs to vascular niches distributed in the coronary circulation and myocardial niches located in the muscle compartment needs to be established (205, 206). This requires the recognition whether BMPCs (Y-chromosome, CD34, CD45, CD133, CD14) are connected by junctional and adhesion proteins (a) to SMCs, ECs and adventitial cells in vascular niches of coronary arteries and capillary structures; and (b) to cardiomyocytes and fibroblasts in myocardial niches. The engrafted male PCs are expected to be c-kit-positive KDR-positive in vascular niches and c-kit-positive KDR-negative in myocardial niches. If BMPCs continuously populate the myocardium, these cells have to possess one fundamental property: they have to be able to divide symmetrically and asymmetrically. The niche microenvironment regulates stem cell division and the generation of a committed progeny and, thereby, controls the size of the PC compartment and the number of parenchymal and non-parenchymal cells within the organ. Symmetric division generates two daughter stem cells and asymmetric division generates one daughter stem cell and one daughter committed cell (207-209). The inhomogeneous intracellular distribution of specific proteins including Numb, α-adaptin and members of the Notch pathway condition symmetric and asymmetric division (210-213). Cells that receive Numb become unresponsive to Notch while Numb-negative cells retain their responsiveness to Notch and adopt the phenotype associated with Notch activation (213, 214). Thus, asymmetric partitioning of gene products at mitosis governs cell fate. The recognition whether male BMPCs reach the myocardium, accumulate in vascular and myocardial niches and divide symmetrically and asymmetrically provides evidence in favor of the bone marrow origin of cardiac PCs. Our data indicate that human VPCs and MPCs divide symmetrically and asymmetrically in vivo and in vitro (1; FIG. 20).

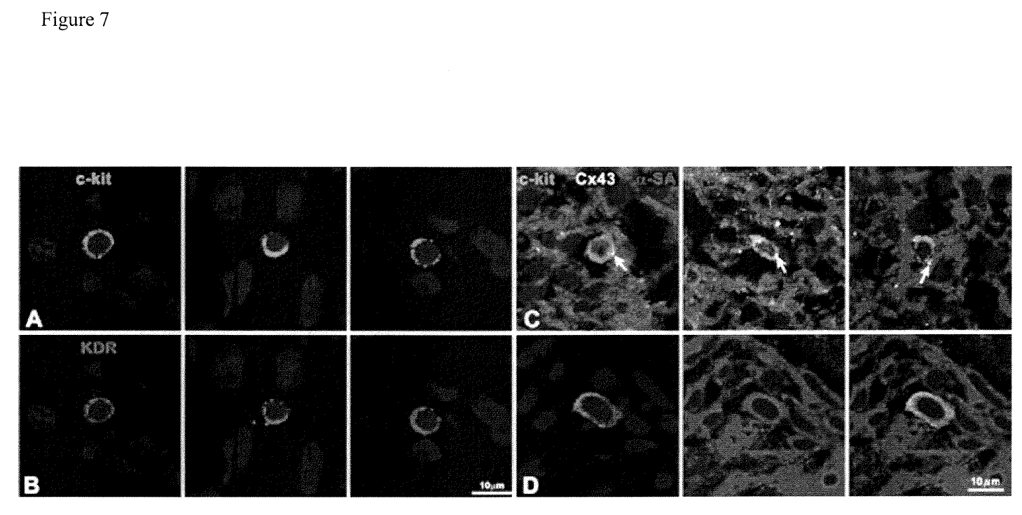

To obtain additional information, the analysis of the adult human heart is complemented with the identification of vascular and myocardial niches in developing human coronary vessels and muscle compartment in prenatal and early post-natal life. Our data show that c-kit-positive KDR-negative MPCs and c-kit-positive KDR-positive VPCs have been found in the developing human myocardium (FIG. 7). The demonstration that c-kit-positive KDR-positive cells are stored in vascular niches and c-kit-positive KDR-negative cells are clustered in myocardial niches strengthens the notion of the non-bone marrow origin of these PCs. Cells committed to the vascular lineages which retain the c-kit and KDR epitopes (ECs: c-kit, KDR, Ets1; SMCs: c-kit, KDR, GATA6) and cells committed to the myocyte lineage which express only the c-kit epitope (c-kit, Nkx2.5, MEF2C) may provide a linear relationship between each PC category and its progeny. However, these data do not exclude that the bone marrow contributes partly to cardiac development.

The transcriptional profile of VPCs, MPCs and BMPCs is assessed to establish shared and distinct genotypic properties among these three cell populations (101-104). Circulating EPCs have the ability to form coronary vessels, raising the possibility that EPCs may constitute the most likely cell population capable of replenishing vascular niches and preserving the VPC pool in the coronary circulation. Thus, the analysis of EPCs has been included. By comparing gene expression patterns, common or unique genes involved in self-renewal, multipotentiality and lineage specification may be identified (101, 102).

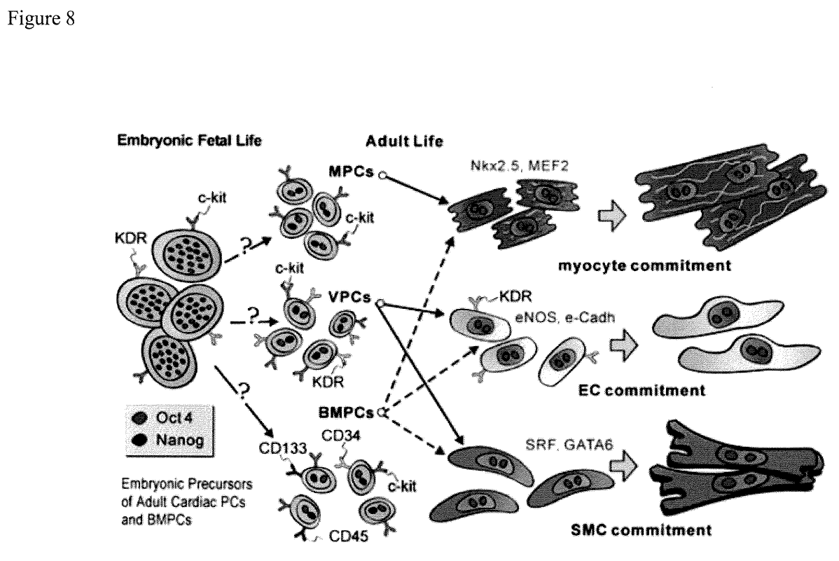

The analysis of the transcriptional profile of PCs addresses two fundamental objectives: a) To identify the genes that characterize undifferentiated VPCs, MPCs and BMPCs; and b) To identify the silencing and upregulation of genes with differentiation of each PC class into myocytes, SMCs and ECs. With this approach, the critical regulators of sternness and commitment of PCs are determined. Oct4 and Nanog may govern the primitive state of VPCs, MPCs, and BMPCs. Conversely, repression of Oct4 and Nanog favors differentiation which is dictated by the expression of lineage specific genes: Nkx2.5 and MEF2 condition myocyte commitment, eNOS and E-cadherin regulate EC commitment, and SRF and GATA6 modulate SMC commitment (FIG. 8). In all cases, PC differentiation is coupled with the loss of the surface epitopes which define each PC category. Stemness of each PC may be preserved only in part by the same set of genes. Similarly, the commitment of VPCs, MPCs and BMPCs to the myocyte, EC and SMC lineages may involve different groups of genes

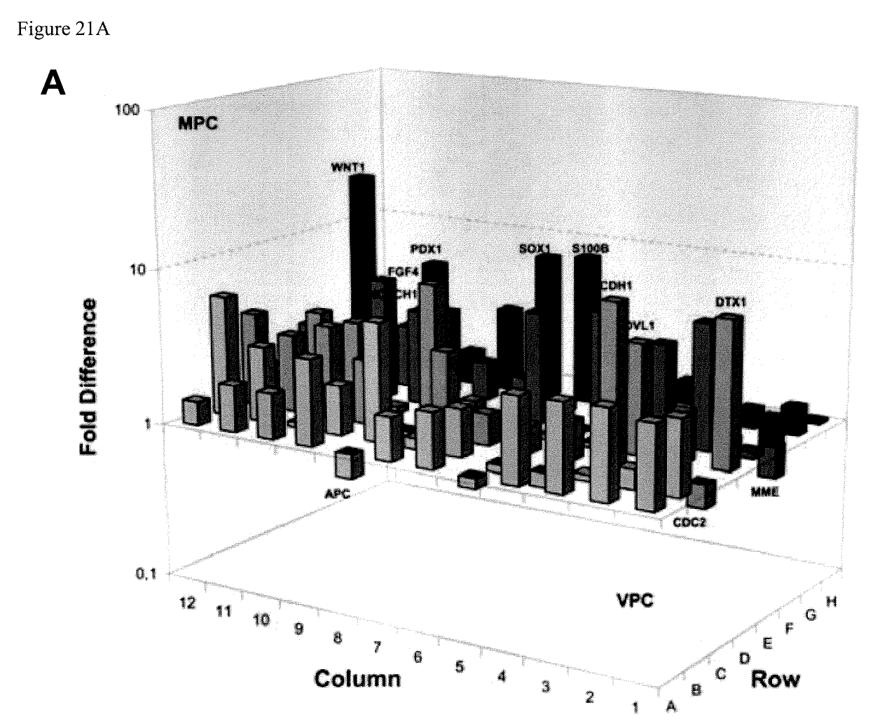

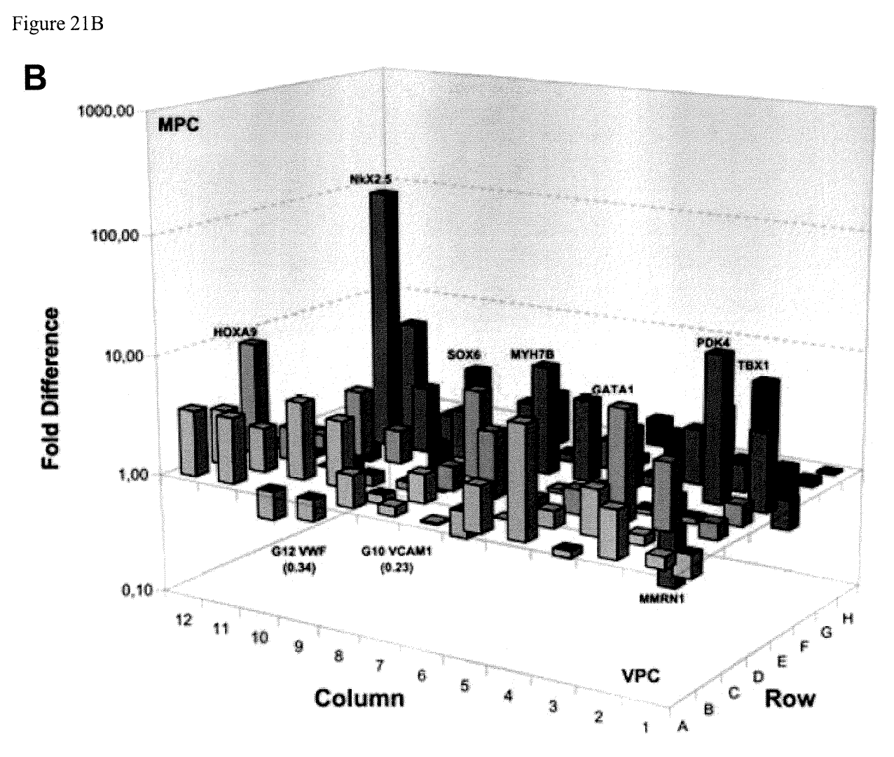

Although the majority of genes may be similarly regulated in these PC populations, cell-type-specific gene expression could be documented and these differentially expressed genes may reflect distinct biological properties. Changes in gene expression in each PC class with the phenotype (proteins) and functional state (differentiation) of the cells are correlated. Our data indicate that striking differences exist between VPCs and MPCs in the quantity of mRNA for genes involved in sternness and commitment (FIG. 21). These data were obtained by real-time RT-PCR array that represents a valid alternative strategy to oligonucleotide microarray. The PCR array allows us to analyze quantitatively the expression of a restricted panel of genes with SYBR Green-optimized primer assays. We employ an array containing a panel of stem cell related genes from SuperArray. Also, we designed a panel of lineage-related genes (see Table 1 below).

TABLE 1
Panel of lineage-related genes.

| | |
|---|---|
| GENE | Unigene |

Stem Cells

| | |
|---|---|
| ABCB1 | Hs.489033 |
| ABCG2 | Hs.480218 |
| ATXN1 | Hs.43796l |
| KIT | Hs.479754 |
| ISL1 | Hs.505 |

CPCs

| | |
|---|---|
| ANGPT4 | Hs.278973 |
| CER1 | Hs.246204 |
| CFC1 | Hs.507542 |
| DKK1 | Hs.40499 |
| FOXH1 | Hs.652162 |
| FRZB | Hs.128453 |
| GATA1 | Hs.765 |
| JAG1 | Hs.774012 |
| MYOCD | Hs.587641 |
| NOTCH2 | Hs.487360 |
| POU5F1 | Hs.749184 |
| SNAI2 | Hs.360174 |
| SRF | Hs.520140 |

Embryonic heart

| | |
|---|---|
| FGF8 | Hs.57710 |
| HAND1 | Hs.152253l |
| HAND2 | Hs.388245 |
| MESP1 | Hs.447531 |
| MESP2 | Hs.37311 |
| TBX1 | Hs.173984 |
| TBX2 | Hs.531085 |
| TBX20 | Hs.404167 |
| TBX5 | Hs.351715 |
| ACTC1 | Hs.118127 |
| ATP1A2 | Hs.34114 |
| BMP2 | Hs.73853 |
| DKK2 | Hs.211869 |
| FGF10 | Hs.654499 |

Myocyte markers

| | |
|---|---|
| FOXN1 | Hs.239 |
| GATA4 | Hs.243987 |
| KCNJ0 | Hs.619400 |
| MB | Hs.517586 |
| MEF2A | Hs.258675 |
| MEF2C | Hs.654474 |
| MYH7 | Hs.278432 |
| MYL4 | Hs.463300 |
| MYOD | Hs.181768 |
| MYOM2 | Hs.443683 |
| NKX2-5 | Hs.54473 |
| NPPB | Hs.219140 |
| PDK4 | Hs.8364 |
| PROX1 | Hs.585369 |
| TAGLN | Hs.632099 |
| TNNT2 | Hs.533613 |

Vascular markers

| | |
|---|---|
| ACE | Hs.654434 |
| ACTA2 | Hs.500483 |
| CALD1 | Hs.490203 |
| CD14 | Hs.163667 |
| CD34 | Hs.374990 |
| CDH5 | Hs.76206 |
| CNN2 | Hs.651923 |
| COL4A2 | Hs.508716 |
| EPAS1 | Hs.468410 |
| FLT1 | Hs.654360 |
| GATA6 | Hs.514746 |
| ICAM2 | Hs.431460 |
| KDR | Hs.479756 |
| KLF4 | Hs.376206 |
| KLF5 | Hs.508234 |
| LOB2 | Hs.23746 |
| MMRN1 | Hs.260107 |
| MYH11 | Hs.460109 |
| NOS3 | Hs.647092 |
| PECAM1 | Hs.514412 |
| SELE | Hs.89546 |
| SMAD4 | Hs.75862 |
| SMAD7 | Hs.465067 |
| SMTN | Hs.119098 |
| TAL1 | Hs.525198 |
| TEK | Hs.89840 |
| TGFB | Hs.645227 |
| TGFBR1 | Hs.494622 |
| VCAM1 | Hs.109225 |
| VEZF1 | Hs.694720 |
| VWF | Hs.440348 |

Fibroblasts

| | |
|---|---|
| COL1A1 | Hs.172928 |
| COL1A2 | Hs.489142 |
| COL3A1 | Hs.443625 |

Epigenetics

| | |
|---|---|
| HDAC2 | Hs.3352 |
| HDAC3 | Hs.519632 |
| HDAC4 | Hs.20516 |
| HDAC7 | Hs.200063 |
| CREBBP | Hs.459759 |
| HOXA9 | Hs.059350 |

Others

| | |
|---|---|
| MYH7B | Hs.414122 |
| SOX6 | Hs.368226 |
| ROD1 | Hs.269388 |
| CFL2 | Hs.180141 |
| IGF1R | Hs.643120 |

Myocardial samples from 10 patients, 30-50 years of age, with modest coronary artery disease and no signs of cardiac failure are employed to assess gene expression of VPCs and MPCs and their functional properties. Similar studies are performed in BMPCs obtained from 10 patients 30-50 years of age.

### Specific Methods

Human cardiac chimerism. Autopsy samples of myocardium of female patients that received sex-mismatched bone marrow transplantation are examined. The male genotype is assessed by FISH for the Y-chromosome (59, 75). The number of X-chromosomes is also measured to evaluate fusion events (1, 59, 68, 70, 86, 89). To determine whether male cells contribute to the formation of vascular and myocardial niches, the presence of gap and adherens junctions between Y-chromosome-positive cells and Y-chromosome-negative cells is assessed (206). If cells of bone marrow origin home and engraft into the wall of coronary arteries, the expression of connexins (type 43, 45, 40, 37) and cadherins (VE-, N-, R-, T-) is expected to occur at the interface between migrated male cells and resident female ECs, SMCs and adventitial fibroblasts. If cells actively translocate to the muscle compartment, the expression of connexins 43 and 45 and N-cadherins should be found between male cells and resident myocytes and fibroblasts (206). Male cells within the niches may express surface epitopes of BMPCs (CD34, CD133, CD45, CD14) or adopt the phenotype of VPCs (KDR, c-kit) or MPCs (c-kit only). Dividing Y-chromosome-positive cells are identified by phospho-H3. The distribution of Numb and α-adaptin are determined (see FIG. 20). To establish male cell differentiation, nuclear and cytoplasmic proteins specific of myocytes (Nkx2.5, GATA4, α-actinin, α-sarcomeric actin, myosin heavy chain), SMCs (SRF, GATA6, α-SMA, SM heavy chain, calponin) and ECs (Vezf1, Ets1, eNOS, E-cadherin, CD31, vWf) are analyzed by confocal microscopy (1, 11, 58, 59, 68, 70, 82, 86, 89).

Highly polymorphic short tandem repeats (STR) and VNTR analysis. DNA is isolated from cardiac samples of the recipient to identify loci of simple repetitive DNA sequences that vary extensively in their repeat number among individuals (200-204). Detection of three of four distinct polymorphisms in the recipient indicates chimerism.

Human VPCs and MPCs. Myocardial samples (n=10) are obtained from patients undergoing heart surgery. VPCs and MPCs are harvested by enzymatic dissociation (1) and single cell suspension characterized by FACS and deposited in individual wells to obtain multicellular clones.

Human bone marrow. Two populations of bone marrow cells are employed: (a) c-kit-positive BMPCs; and (b) EPCs. For BMPCs (82, 83, 86), 10 samples from patients with hematological diseases in which there is no bone marrow involvement are studied. Bone marrow, ˜4 ml, is obtained. After density gradient separation, mononuclear cells are collected and incubated with a cocktail of bead-conjugated antibodies specific for lineage-epitopes of bone marrow cells. After lineage depletion, the unsorted cells are incubated with bead-conjugated c-kit antibody (clone AC126). Enrichment is evaluated by cytospin and FACS with a c-kit antibody (clone A3C6E2).

Samples for molecular biology. Undifferentiated BMPCs are used immediately after c-kit sorting. Clonogenic VPCs and MPCs and non-clonogenic VPCs and MPCs as well as BMPCs are cultured in “generic” differentiating medium and in “predominantly” EC-producing, SMC-producing or myocyte-producing medium. The “generic” differentiation medium consists of F12 supplemented with 10⁻⁸ M dexamethasone (1). For SMC differentiation, PCs are grown in collagen IV-coated dishes in F12 medium supplemented with 1 ng/ml recombinant TGFβ1. For EC differentiation, PCs are seeded in methylcellulose plates with 100 ng/ml recombinant VEGF. For myocyte differentiation, PCs are co-cultured with myocytes from β-actin-EGFP mice (1). Cell differentiation and function are assessed in parallel cultures.

FACS. Aliquots of VPCs, MPCs, BMPCs and EPCs are incubated with primary antibody against c-kit and KDR and other markers (1). Antigens for bone marrow cells: CD2 (T cells, Natural Killer cells), CD3 (T cells), CD8 (T cells), CD14 (monocytes), CD16 (neutrophils, monocytes), CD19 (B cells), CD20 (B cells), CD24 (B cells), CD41 (hematopoietic cells), CD34 (HSCs, EPCs), CD45 (leukocytes, mast cells), CD133 (HSCs, EPCs), glycophorin A (erythrocytes); for vascular cells: GATA6 (SMC transcription factor), Ets1 (EC transcription factor), Tie-2 (angiopoietin receptors), VE-cadherin (cell adhesion molecule), CD62E/E-selectin (cell adhesion molecule), α-SM-actin (contractile protein), CD31 (PECAM-1), vWF (carrier of factor VIII); for myocytes: GATA4 (cardiac transcription factor), Nkx2.5 and MEF2C (myocyte transcription factors), α-sarcomeric-actin (contractile protein).

Clonogenicity and growth of VPCs and MPCs. Cloning efficiency is determined (1, 6, 11). Clonogenic cells are counted daily and population doubling time is calculated (215). The fraction of cycling and non-cycling cells is determined by BrdU and Ki67 labeling (1, 6, 11).

Immunocytochemistry. Undifferentiated and differentiated VPCs, MPCs, BMPCs and EPCs are identified by the expression of lineage-related markers for SMCs (SRF, GATA-6, α-SM-actin, SM-heavy chain, calponin), ECs (Vezf1, Ets1, CD31, eNOS, vWF, VE-cadherin) and myocytes (Nkx2.5, MEF2C, α-sarcomeric-actin, α-actinin, troponin I, troponin T, cardiac myosin heavy chain, connexin 43, N-cadherin).

Cellular physiology. Mechanics and Ca2+ transients: Myocytes are stimulated by platinum electrodes. Changes in cell length are quantified by edge tracking. Simultaneously, Fluo 3-fluorescence is excited at 488 nm. Different rates of stimulation and different extracellular Ca2+ concentrations are examined (7, 11, 216, 217). Electrophysiology: Data are collected by means of whole cell patch-clamp technique in voltage- and current-clamp mode and by edge motion detection measurements. Voltage, time-dependence and density of L-type Ca2+ current are analyzed in voltage-clamp preparations. Additionally, the T-type Ca2+ current is assessed; this current is restricted to young developing myocytes (218). Also, the relationship between cell shortening and action potential profile is determined in current-clamp experiments (89, 219-222). For SMC differentiation, cells are cultured in the presence of TGF-β1 (223) and their properties defined (224-227). For EC differentiation, colonies taking up Dil-Ac-LDL and binding lectin are identified (228).

RT-PCR array. Undifferentiated and differentiated VPCs, MPCs, BMPCs and EPCs are resuspended in Trizol. RNA is extracted and processed at the Superarray Facility.

Western blotting. The expression of selected genes is confirmed at the protein level (6, 7, 58, 59).

Data Analysis. For each of the 10 specimens of human myocardium (n=10 patients) an average 5 expandable clones each for VPCs and MPCs is analyzed. From each clone, ˜8×10⁶ cells are obtained (1). In each case, for each PC class, ˜40×10⁶ cells are generated. Approximately, 23 population doublings (PDs) are necessary to collect ˜8×10⁶ cells from a single founder cell (1); ˜13 PDs are required to develop ˜40×10⁶ non-clonogenic VPCs and MPCs. Cells from individual clones are pooled to obtain a more representative cell sampling in each patient. Clonogenic human cardiac PCs can be easily expanded to this quantity (1).

### Example 2

### Epigenetic Mechanisms in the Control of Gene Expression in Human VPCs, MPCs, and BMPCs

The experiments in this Example are designed to determine whether epigenetic mechanisms condition the growth and differentiation of human VPCs, MPCs and BMPCs.

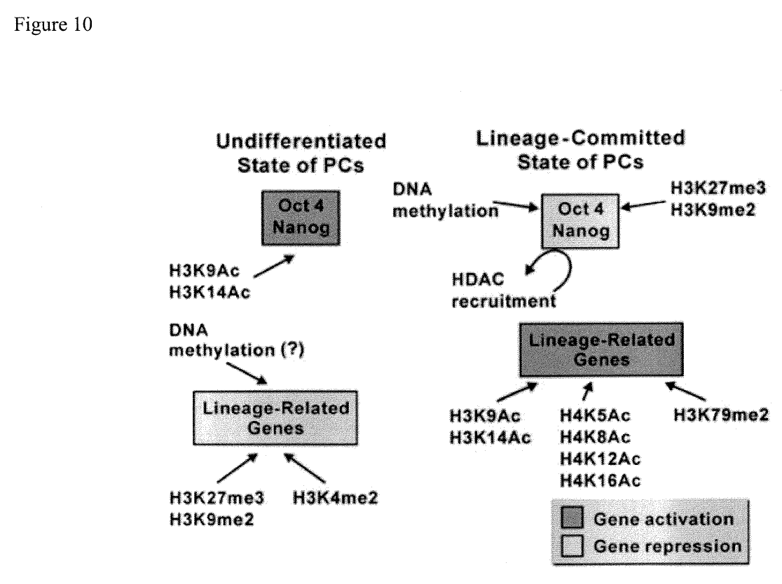

The molecular properties of undifferentiated and committed VPCs, MPCs and BMPCs are defined. A common event that has to occur with differentiation of PCs is the repression of stemness-related genes. The transition from sternness to a differentiated phenotype may be governed by upregulation and downregulation of specific groups of genes (95, 98, 103, 112) which are epigenetically regulated by DNA methylation and histone methylation and acetylation (107-109, 119-121). The undifferentiated state of human PCs may be sustained by expression of the stemness-related genes, Oct4 and Nanog, and silencing of lineage-related genes (see below). This transcriptional program is proposed to be controlled by a bivalent chromatin configuration in which the repressive marks H3K9me2 and H3K27me3 coexist with the activating mark H3K4me2. The promoter of Oct4 and Nanog may also be highly enriched in H3K9Ac which would promote transcription (FIG. 10).

The acquisition of a committed cell phenotype may be prompted by DNA methylation of the promoter of Oct4 and Nanog and/or loss of histone acetylation in the same promoter regions through activation of HDACs. The preferential commitment of MPCs to the myocyte phenotype may be mediated by activation of the transcription factor Nkx2.5 which is followed by upregulation of MEF2 transcription factors and ultimately synthesis of contractile proteins (FIG. 10). During cardiac development, the expression of Nkx2.5 involves a complex sequence of histone acetylation of regulatory modules located in the promoter region (233). In a comparable manner, the early commitment of MPCs to the myocyte lineage may require histone acetylation of the proximal enhancers G-S and AR2 of Nkx2.5 promoter followed by activation of the distal enhancers UH5 and UH6. This would indicate that the formation of myocytes from adult MPCs mimics embryonic cardiomyogenesis (68, 234, 235). Later in the differentiation process, histone acetylation of promoter regions of MEF2 may upregulate a variety of MEF2-dependent genes (236) subsequently resulting in the accumulation of muscle specific proteins.

Class IIa HDACs repress MEF2 transcription by interacting with MADS-domains bound to the promoter of MEF2 (176-179) and by recruiting class I HDACs (177, 178). Thus, MPC differentiation may be regulated by dissociation of class I and class IIa HDACs and acetylation of the MEF2 promoter. The interaction between HDACs and MEF2, however, may be more complex than originally thought. Class I and class IIa HDAC inhibitors have opposite effects on cardiac hypertrophy; they may influence different groups of MEF2 effector genes (237, 238). Class I HDACs may inhibit anti-hypertrophic genes while class IIa HDACs may repress pro-hypertrophic genes raising the possibility that these two families of deacetylases have differing function on MPC differentiation (161, 237, 238).

The commitment of VPCs to the SMC lineage may be mediated by activation of the transcription factors SRF and GATA6 and then by expression of SMC contractile proteins. During differentiation of VPCs into SMCs, the chromatin structure of the promoter of SRF is expected to change from a non-permissive configuration to a transcription-permissive configuration. The SRF promoter of VPCs may contain heterochromatic (repressive) histone modifications consisting of H4K20me2, H3K9me3 and H3K27me3 (239-241). Upon VPC commitment, enrichment in euchromatic (activating) histone modifications may occur and this may involve H4K5Ac, H4K8Ac, H4K12Ac and H4K16Ac together with H3K4me2, H3K9Ac, H3K14Ac and H3K79me3 (240, 241-243). If VPCs differentiate into non SMC-lineages the repressive marks H3K9me3 and H3K27me3 in the SRF promoter are expected to persist. Similar epigenetic mechanisms may regulate the expression of GATA6. With commitment, GATA6 transcription may be mediated by acetylation of histone H3 and H4 and accumulation of H3K4me2 (244).

Differentiation of VPCs into ECs may be dictated by eNOS and E-cadherin expression (20, 21). As shown in FIG. 11, the eNOS promoter is epigenetically regulated by DNA methylation. Consistent with the developmental expression of eNOS, methylated CpG sites accumulate in the eNOS promoter of undifferentiated EPCs, mesangioblasts and CD34-positive BMPCs while unmethylated CpG sites are present in committed ECs. Alternative epigenetic mechanisms that may modulate eNOS expression consist of histone acetylation (H3K9Ac, H4K12Ac) and di- or tri-methylation of histone H3 (H3K4me2, H3K4me3). The differentiation of VPCs into non-EC lineages may involve DNA methylation of the eNOS promoter which may favor the recruitment of HDACs inhibiting eNOS expression (245). A similar epigenetic regulation may control E-cadherin expression. Silencing of the E-cadherin promoter in undifferentiated VPCs may be conditioned by DNA methylation, repressive histone methylation (H3K9me2, H3K27me3) and/or hypoacetylation of histone H3 and H4. With commitment, transcription of E-cadherin may be promoted by HDAC dissociation and accumulation of H3K4me2 (246).

Although to a lesser extent than VPCs, MPCs have the ability to form vascular cells. It is important to establish whether the gene promoters involved in vascular commitment are held in a repressive state in MPCs favoring the differentiation of this PC class into cardiomyocytes. In a similar manner, the greater efficiency of VPCs than myocytes to generate SMCs and ECs may be dictated by a tighter chromatin configuration in the promoter regions of myocyte-specific genes, such as NKx2.5 and MEF2. For BMPCs, the genes that condition the acquisition of the myocyte, SMC and EC phenotype and, subsequently, the epigenetic mechanisms that maintain the plasticity of BMPCs and dictate their cardiovascular lineage specification can be identified.

Myocardial samples from 10 patients, 30-50 years of age, with modest coronary artery disease and no signs of cardiac failure are employed to define the epigenetic mechanisms that regulate stemness and commitment of VPCs and MPCs. Similarly, BMPCs are obtained from 10 patients 30-50 years-old to identify the epigenetics of BMPC plasticity.

ChIP assays are performed to identify the specific histone acetylation and methylation pattern in the promoter regions of the genes involved in stemness (Oct4, Nanog) and differentiation (Nkx2.5, MEF2, eNOS, E-cadherin, SRF, GATA6) of PCs (see FIG. 10). Both genome-wide and promoter-specific results have been collected in mouse ESCs (FIG. 13).

ChIP assays are performed to determine whether the promoter regions of Nkx2.5, MEF2, eNOS, E-cadherin, SRF and GATA6 contain H3K9Ac and H3K14Ac in VPCs, MPCs and BMPCs. Similarly, the presence of H3K79me2 in the regulatory regions of these lineage-related genes are assessed. It is noteworthy that shear stress induces H3K79me2 which, in turn, appears to be linked to acquisition of cardiac cells lineages.

In summary, our data show that epigenetic changes of histones are present in human MPCs and VPCs. Activating and repressing marks are found in various combinations in these human cells. The inactivating marks, H3K27me3 and H3K9me2, and the activating mark, H3K4me2, co-exist in MPCs and VPCs indicating that the chromatin structure of these cardiac PCs has a dynamic configuration and possesses a certain level of plasticity (107-113, 147, 148). At times, di-methylation of histone H3 at lysine 79 was seen in human MPCs but not in VPCs. H3K79me2 is upregulated by shear stress and this epigenetic change is coupled with activation of the VEGFR2 promoter inducing cardiovascular differentiation of ESCs (146).

### Specific Methods

DNA methylation. DNA methylation of the promoter regions of target genes is measured by the sodium bisulfite genomic sequencing technique (247, 248; FIG. 14). Genomic DNA is treated with sodium bisulfite which converts all unmethylated cytosines into uracil. DNA is then amplified by nested PCR with primers specific for methylated and unmethylated CpG sites located in the promoters of the genes of interest. PCR products are sequenced, the proportion of methylated cytosines quantified and their position in the promoters established.

Western blotting. Genome-wide methylation and acetylation of histone 3 and histone 4 are analyzed. Protein lysates are obtained with Laemmli buffer containing β-mercaptoethanol. Proteins are separated on 15% SDS-PAGE, transferred onto nitrocellulose and exposed to specific antibodies against different histone modifications (H3K9me2/3, H3K27me3, H3K4me2/3, H3K79me3, H4K20me2, H4K5Ac, H4K8Ac, H4K12Ac, H4K16Ac, H3K9Ac, H3K14Ac). Loading conditions are determined by β-actin expression (6, 7, 58, 59). In a similar manner, HDAC expression is quantified in total cell lysates and in nuclear and cytoplasmic lysates (249, 250).

Chromatin immunoprecipitation (ChIP). To map the location of modified histones on the promoters of specific genes, formaldehyde-cross-linked DNA is fragmented by sonication and pulled down with antibodies specific for the histone modifications listed above (251). Immunoprecipitated chromatin is recovered and the cross-linking reversed (251). The promoter regions of the gene of interest (i.e. Oct4 and Nanog for undifferentiated cells; Nkx2.5 and MEF2 for cardiomyocytes; SRF and GATA6 for SMCs; eNOS and E-cadherin for ECs) are recognized by PCR with specific primers.

ChIP-on-Chip. In a subset of patients, differences in the transcriptional profile of VPCs, MPCs and BMPCs may not be apparent since we are testing by RT-PCR array 84 stemness-related genes and 84 lineage-related genes. In these cases, ChIP-on-Chip is used to identify a large number of DNA sequences associated with the modifications of histones detected at genome wide level. This technique involves ChIP followed by the simultaneous detection of the DNA sequences co-immunoprecipitated with the protein of interest by DNA array. A chip containing 600 promoters of cardiovascular genes and 200 promoters of cell cycle-related genes will be employed. ChIP is performed with 5×10⁶ cells. After cross-linking reversal, proteins are removed from the samples and DNA is extracted and purified. Then, DNA is amplified by ligation-mediated PCR (LM-PCR), labeled with fluorophores and employed in the hybridization with the promoter microarray.

### Example 3

### Effects of Aging and Heart Failure on the Epigenetic Regulation of Gene Expression in Human VPCs, MPCs, and BMPCs

The purpose of this Example is to determine whether aging and heart failure promote epigenetic changes which negatively affect the function of human VPCs, MPCs and BMPCs.

Epigenetic modifications are important determinants of cellular senescence, organism aging and heart failure (161, 252, 253). Epigenetic changes of PCs may occur and play a role in human myocardial aging. Similarly, ischemic and non-ischemic cardiomyopathy and the duration and severity of the disease state may have profound implications on PC function. This information is of great importance in the application of PC therapy to patients. To develop strategies relevant to the management of the aging myopathy and heart failure in humans, the effects of age, gender, disease history and clinical conditions on PC behavior are determined. The assumption has been made that aging effects on PCs are comparable to those induced by a prolonged and sustained overload on the heart. This possibility has been shown to be valid in animal models (216, 254) and humans (58, 59, 255) suggesting that pathologic conditions result in premature PC aging.

Samples are obtained from approximately 200 patients undergoing cardiac surgery. These patients are commonly studied by echocardiography and/or NMR. The age, sex, history of the patients, primary disease and its evolution together with the functional and anatomical parameters of the diseased heart are coded and the code is broken when groups of ˜40 patients each have been studied. Bone marrow samples from the sternum and excised ribs of patients undergoing cardiac surgery are obtained to have a direct comparison of BMPCs, VPCs and MPCs in the same individuals. Importantly, different classes of bone marrow cells are currently being employed in the treatment of acute and chronic heart failure in humans (157, 158, 256). BMPCs harvested from patients of different age without cardiac diseases are also analyzed. The age range available for both the heart and bone marrow is ˜20 to 85 years. Thus, the properties of VPCs, MPCs and BMPCs are determined.

Chronological age may not represent the only important parameter in the comparison between individuals of different ages and cardiac pathology. There are several variables of the aging process that cannot be easily quantified but, perhaps, have dramatic consequences on organ and organism aging and heart failure. Chronological age and biological age do not necessarily coincide and organism and organ age do not necessarily proceed at the same pace (68). Moreover, chronological age of individual cells in an organ is highly heterogeneous being conditioned by the birth date of the individual cells and biological age of cells differs according to the extent of damage that cells have suffered with time. When possible, the epigenetic data on PCs are complemented by the expression of markers of cellular senescence at the single cell level. The senescence-associated protein p16INK4a and telomere length are employed for identification of aged cells within the PC pool (59, 253).

The objectives of this Example are: (a) To measure differences in gene expression of PCs (VPCs, MPCs, BMPCs) obtained from patients at different age and cardiac pathology; (b) To identify gene promoters that undergo DNA hypermethylation and thereby gene silencing with aging and heart failure; (c) To establish whether a histone code of senescent PCs exists with chronological age and is comparable to that found in PCs of younger patients with heart failure; (d) To recognize the gene promoters that show aberrant histone methylation and acetylation in PCs from old individuals and patients with heart failure; (e) To assess whether epigenetic changes affect in a similar or distinct manner each PC class; and (f) To determine whether the epigenetic changes have a functional counterpart interfering with the growth and/or differentiation properties of PCs.

The transcriptional profile of VPCs, MPCs and BMPCs are compared and genes that are consistently downregulated and upregulated with age and heart failure are identified. The changes in gene expression with age and heart failure may be due to epigenetic modifications of their promoters. Gene silencing may depend on aberrant hypermethylation of CpG islands at the level of the corresponding promoter regions. This epigenetic modification typically occurs in cancer cells and affects the promoter of tumor suppressor genes (247, 248). Importantly, this modality of gene silencing involves the promoter of the RecQ helicase that is methylated in a subset of patients affected by Werner syndrome (252), a premature form of organism aging. Other examples of genes with increased promoter methylation with aging include E-cadherin, estrogen receptor and IGF II (252). Gene methylation of the estrogen receptor has been linked to heart disease and development of atherosclerosis (257, 258). The accumulation of methylated CpG islands at the PKC-ε promoter occurs in the heart of babies of crack-cocaine mothers (259). Cocaine-mediated repression of this cardioprotective enzyme may be implicated in the incidence of heart failure and ischemic injury in children exposed to the drug during prenatal life. The age-dependent regulation of the INK4 locus is of particular relevance. The promoter of p16INK4a shows an accumulation of methylated CpG islands in senescent cells in spite of the increased expression of the protein (252). This suggests that an epigenetically-independent upregulation of this cell cycle inhibitor occurs with age.

Alternatively, gene silencing in senescent PCs may depend on the imbalance between activating and inactivating histone marks. An increase in heterochromatic histone modification H4K20me3 is present in aged cells (260). Thus, multiple post-translational modifications of histone H3 and H4 are analyzed to establish whether senescent PCs are characterized by a specific histone code.

Upregulation of specific genes in aging cells may be conditioned by enhanced histone acetylation which in turn may be dictated by decreased deacetylase activity. Typically, SIRT1, a class III HDAC, is downregulated with aging (261) and in senescent cells (262). SIRT1 acts on histone tails mainly catalyzing the removal of acetyl groups from H4K16 and H3K9 (263). Non-histone targets of SIRT1 include p53 and FOXO. The activity and stability of p53 are enhanced by acetylation of multiple lysine residues (264). Conversely, both SIRT1 and HDAC1 deacetylate p53 at lysine 382 decreasing its function (265). Increased p53 acetylation is associated with senescence while the increased activity of SIRT1 extends replicative lifespan of human SMCs. Thus, high level of SIRT1 expression and activity characterize young cells leading to deacetylation of p53, p53 degradation and cell proliferation together with deacetylation of histones and selective gene silencing (266). These epigenetic modifications promote longevity. Conversely, the decrease in SIRT1 expression and activity in aging cells results in hyperacetylation of p53 and growth arrest (266). Additionally, hyperacetylation of histone H1 occurs in old cells and this may favor its own degradation; histone H1 loss leads to the formation of senescence-associated heterochromatic foci and gene silencing (266). These epigenetic lesions promote replicative senescence.

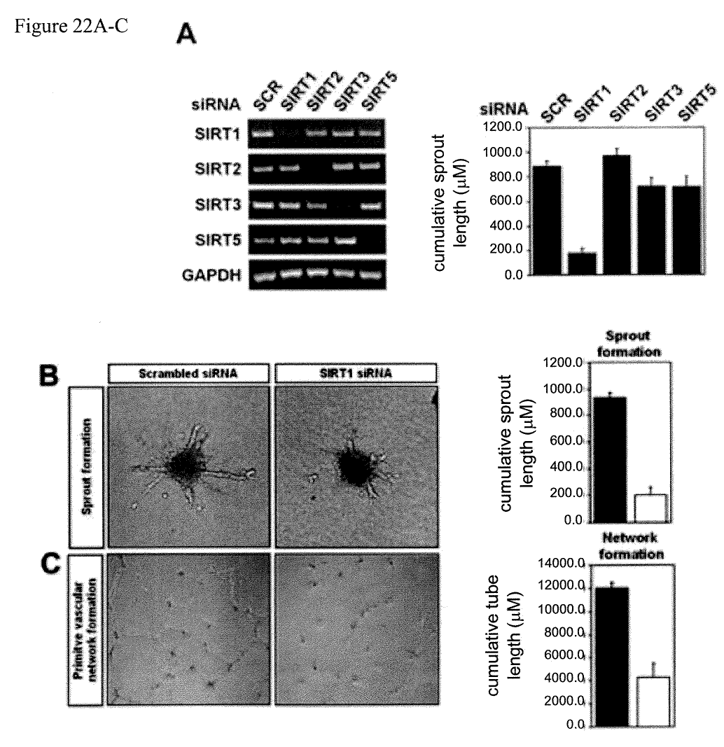

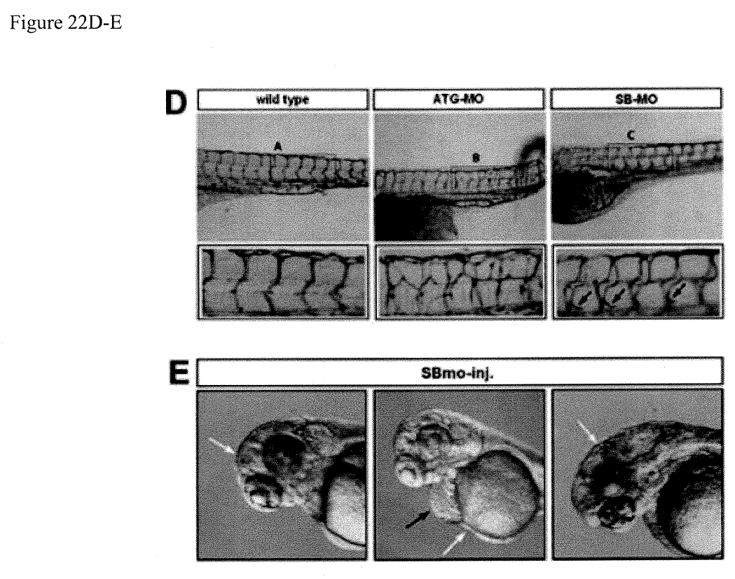

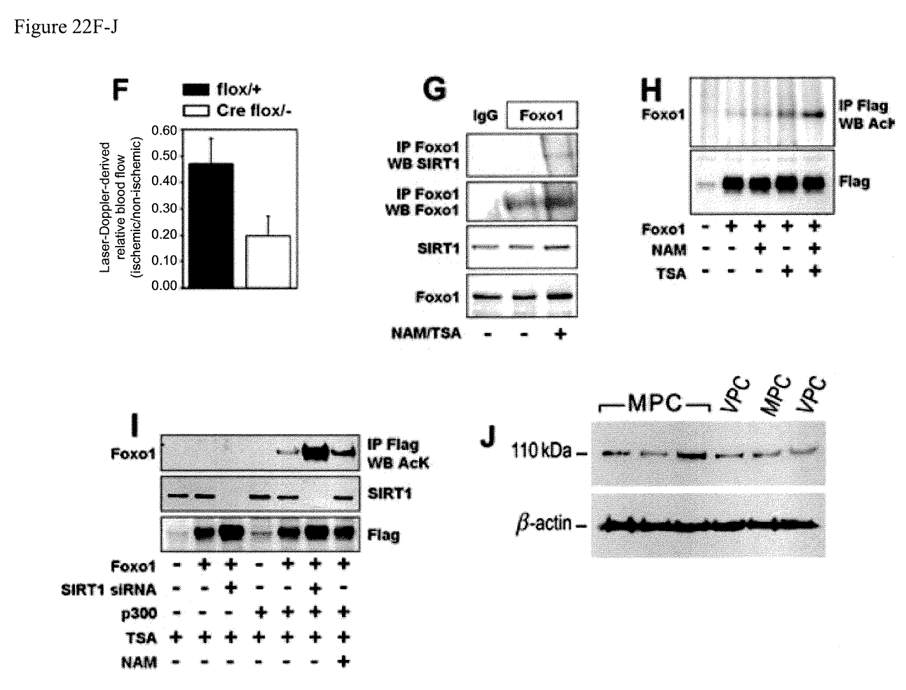

Our data uncover a novel role for SIRT1 as a critical modulator of EC gene expression and postnatal vascular growth (156). SIRT1 is highly expressed in vessels during active growth. Disruption of SIRT1 expression in zebrafish and mice results in defective blood vessel formation and blunts ischemia-induced neovascularization (FIG. 22). This function of SIRT1 is mediated by deacetylation of the forkhead transcription factor FOXO1, a negative regulator of vessel growth. Thus, PCs from old and failing hearts may undergo a decrease in SIRT1, FOXO1 upregulation and defective expression of genes involved in vascular and myocyte growth. Importantly, VPCs and MPCs express SIRT1 (FIG. 22).

### Example 4

### Effect of Epigenetic Modulators on Human VPCs, MPCs, and BMPCs In Vivo

The purpose of this Example is to determine whether epigenetic modulators affect the growth and differentiation behavior of human VPCs, MPCs and BMPCs in vivo.

The objective of this Example is to reactivate the transcription of genes which have been silenced with age and heart failure. Silencing may involve stemness-related genes and/or lineage-related genes with different consequences on the functional behavior of PCs. Repression of Oct4 and Nanog may be characterized by loss of sternness, severely attenuated PC growth or irreversible commitment. Conversely, the inhibition of transcription of Nkx2.5 or MEF2 may be coupled with defective myocyte formation. Gene silencing is dictated by three epigenetic mechanisms: loss of histone acetylation, excessive methylation of histones at repressive sites and DNA methylation.

These epigenetic modifications can be efficiently reverted by inhibition of enzymes that establish the epigenetic marks, i.e., epigenetic modulators. Several molecules capable of interfering with DNA methylation, histone lysine methylation and acetylation are currently available and some of them are being tested clinically (267-269). However, the majority of these compounds affect globally the genome and their effects on gene expression are unpredictable. The use of molecules that alter histone methylation may be particularly challenging. Histone methylation exists both as activating and inactivating marks and it might be difficult to anticipate whether drugs modifying the pattern of global histone methylation have the desired effect. This obstacle may be overcome when molecules acting on specific lysine residues become available. Experimentally, hypoacetylation of histone H3 and histone H4 and loss of methylation at H3K4 have been identified as critical epigenetic mediators of gene silencing (268). Thus, epigenetic modulators that inhibit HDACs or stimulate histone acetyltransferases would represent a valid strategy for the reactivation of gene transcription.

HDAC inhibitors block with variable efficiency HDACs and promote gene transcription by histone acetylation (269). Trichostatin A (TSA) is a class I and II HDAC inhibitor which induces cell cycle arrest and differentiation (269, 270). Of interest, TSA blunts myocardial hypertrophy following pressure overload (271). Novel synthetic compounds such as MS27-275 have been developed; they have an inhibitory function on specific HDACs (269).

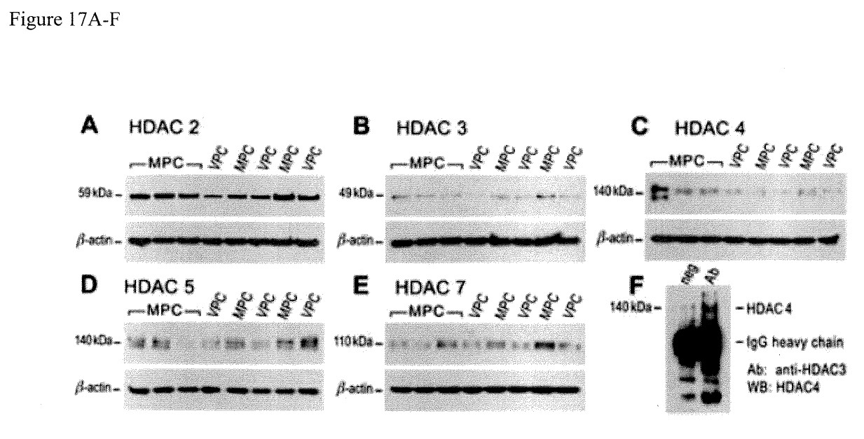

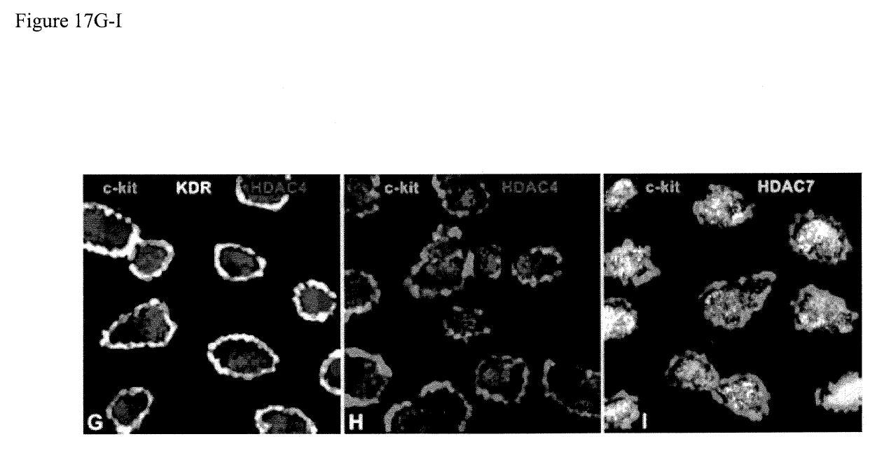

Our data indicate that HDACs are present in human cardiac PCs. MPCs and VPCs express HDAC2-5 and HDAC7 (FIG. 17). As in ESCs (FIG. 18), HDAC4 forms a complex with HDAC3 in MPCs. This protein-to-protein interaction inhibits skeletal myogenesis by interfering with myoblast differentiation (198). Whether this protein complex is implicated in the preservation of sternness of MPCs by preventing cardiomyogenesis is determined by ChIP assay and HDAC inhibitors. The subcellular distribution of HDACs was established by immunofluorescence; in MPCs, HDAC4 is restricted to the nucleus while in VPCs is diffuse. Additionally, HDAC7 is distributed to both nucleus and cytoplasm in MPCs. These observations suggest that HDAC4 has a different function in cardiac PCs. The nuclear localization of HDAC4 in MPCs may result in gene silencing whereas its presence in the cytoplasm of VPCs may promote gene expression. Data obtained in mouse ESCs (FIG. 18) demonstrate that class II HDAC4 and 7 shuttle first to the nucleus and then rapidly back to the cytoplasm after LIF removal. With differentiation and expression of lineage markers, HDACs return to the cytoplasm. Consistently, the activity of HDACs increases early after LIF depletion decreasing with time. This response is inhibited by class I and II HDAC inhibitor, trichostatin A.

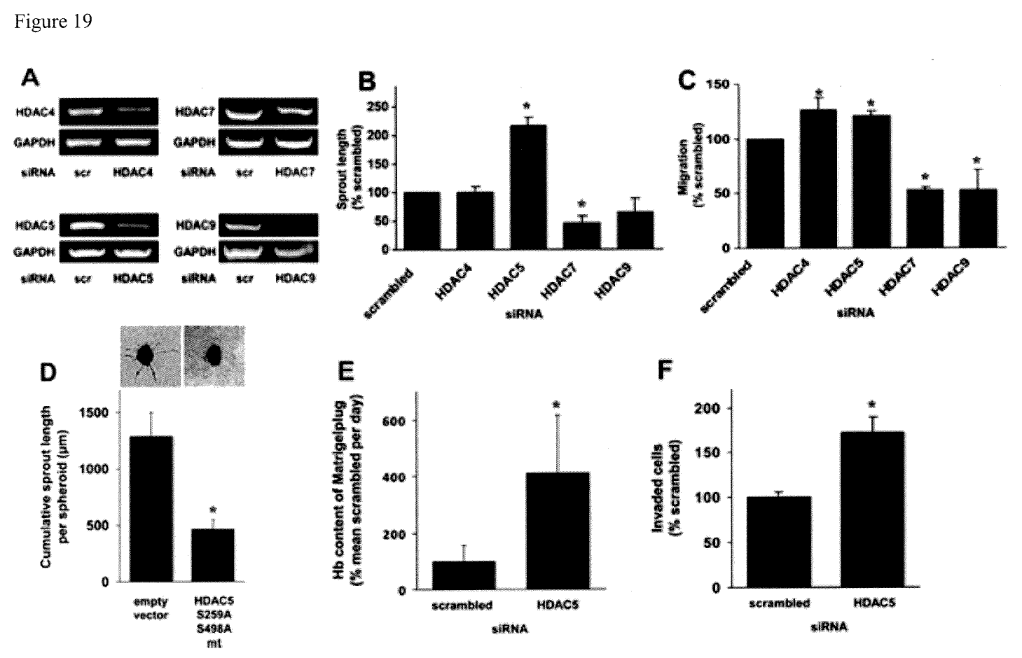

Specific siRNAs against class IIa human HDACs were developed and tested in HUVEC. Our data indicate that this strategy effectively suppresses the mRNA expression of HDAC4, 5, 7 and 9. Importantly, inhibition of HDAC7 interferes severely with the migration and sprout-forming capacity of HUVEC while selective blockade of HDAC5 has the opposite effect (FIG. 19). These siRNAs are used in the characterization of the epigenetic regulation of growth and differentiation of adult human VPCs, MPCs and BMPCs.

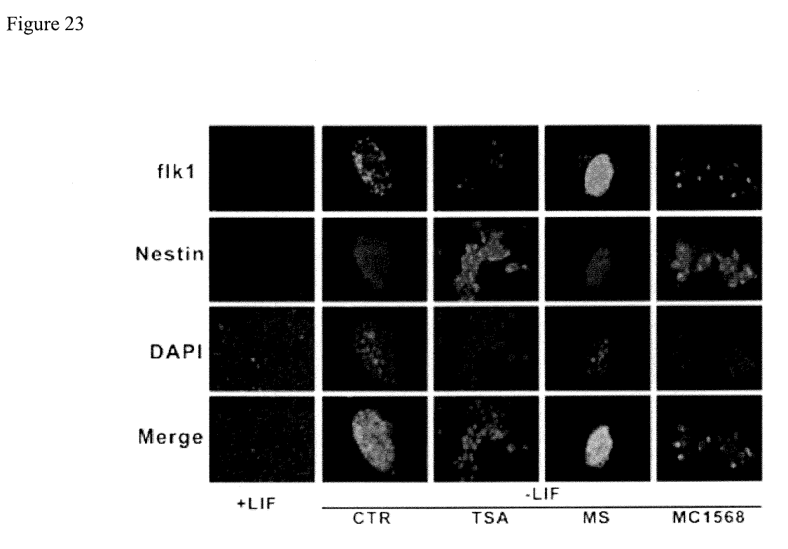

Additionally, our data indicate that the class I HDAC-specific inhibitor MS27-275 triggers differentiation of ESCs to cardiac cell phenotypes (flk1, CD31, SM22, α-SA). Conversely, it opposes neuronal commitment (FIG. 23) suggesting that class II HDACs positively regulate the acquisition of a mesodermal lineage. In this regard, a class II HDAC specific inhibitor MC1568 (272) favors neuronal differentiation and inhibits the cardiac commitment of ESCs (FIG. 23).

Based on these initial observations, protocols aiming at the recognition of factors that reactivate the expression of aberrantly silenced genes in human PC classes are developed. Specific HDAC inhibitors that restore the physiological balance of growth and differentiation of PCs preserving their undifferentiated state or promoting their lineage commitment may be identified. This intervention may enhance the regenerative capacity of old, poorly functioning VPCs, MPCs and BMPCs ultimately favoring their clinical implementation.

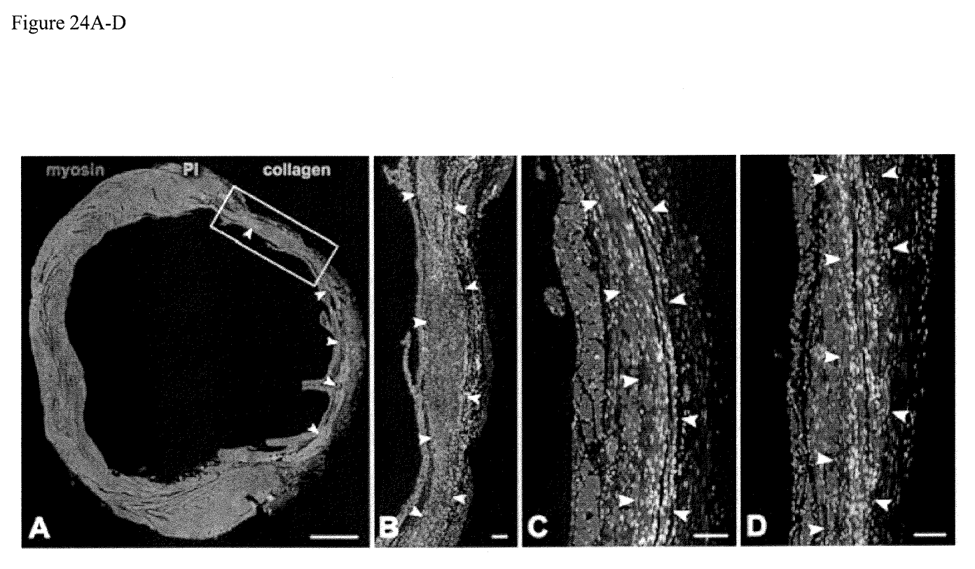

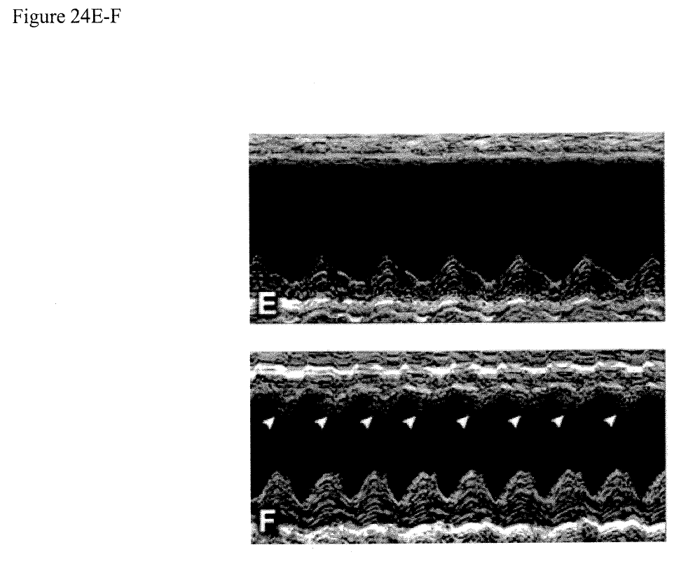

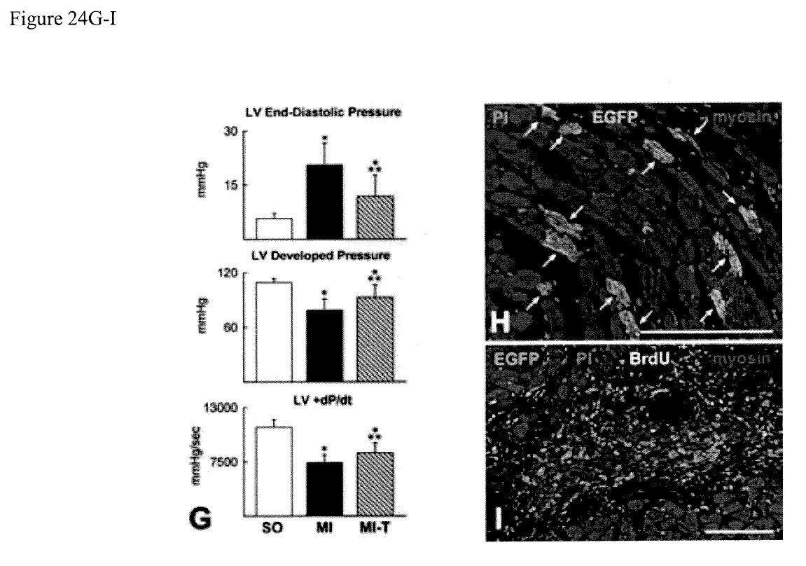

Although questions can be raised concerning the ability of VPCs, MPCs and BMPCs to replace scarred tissue with functional myocardium, our data in the infarcted rat heart suggest the feasibility of this form of cellular therapy. Similarly, results in the aging failing heart indicate that small foci of replacement fibrosis and scattered myocyte death can be repaired by activation of resident PCs (FIG. 24). Untreated old human PC classes and old PCs exposed to distinct HDAC inhibitors may be able to replace scarred infarcted myocardium.

### Specific Methods

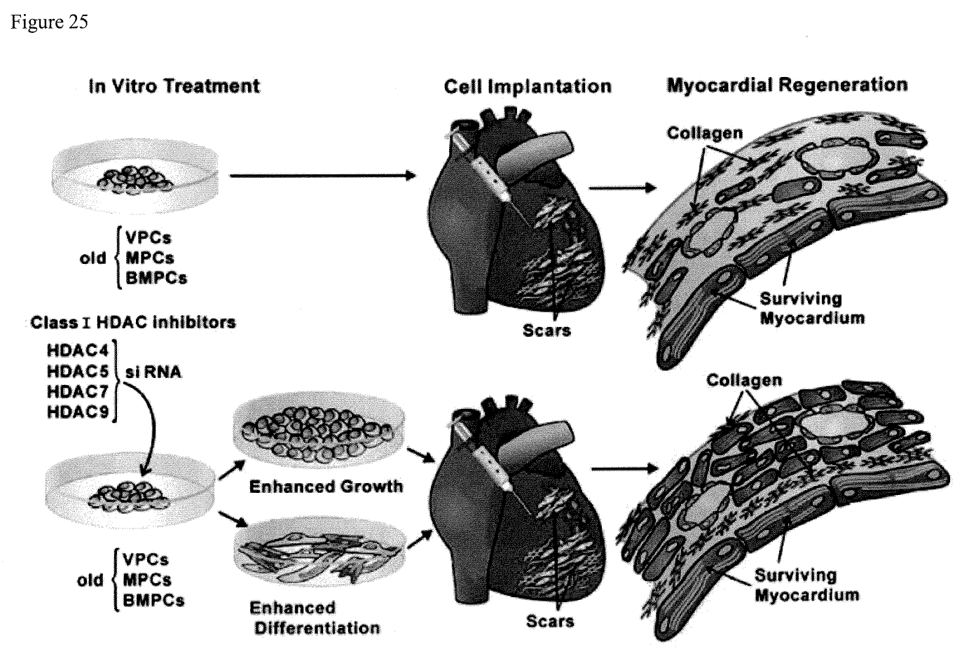

In vitro studies. VPCs, MPCs and BMPCs from the 10 worst cases identified in the first group of 65 patients studied in Example 3 are employed. The baseline studies in Example 3 are complemented in this Example with the analysis of the effects of the class I HDAC-specific inhibitor MS27-275, HDAC4-siRNA, HDAC5-siRNA, HDAC7-siRNA and HDAC9-siRNA on the parameters listed in Example 3. The 5 sets of VPCs, MPCs and BMPCs (n=5 patients) that respond better (increased cloning efficiency and/or differentiation) to HDAC inhibition are tested in vivo (FIG. 25). Controls include untreated PCs and PCs treated with the scrambled sequences of HDAC-siRNA.

Animals. Myocardial infarction is induced in Fisher 344 rats at 3 months of age and PCs are injected 4 weeks later (1, 7, 11); 4 injections of 10,000 cells each are made at the two opposite sides of the scar. Prior to injection cells are infected with a lentivirus carrying EGFP for their subsequent recognition in vivo (1, 89) together with human Alu DNA sequences (1). Myocardial regeneration is evaluated 4 weeks later. Immunosuppression with cyclosporin A is initiated at the time of cell administration and maintained throughout (1). Similarly, Alzet microosmotic pumps (2ML4) that release BrdU continuously for 4 weeks are implanted.

Echocardiography. Echocardiography is performed two days after coronary occlusion and at 2 and 4 weeks. A similar protocol is applied after cell implantation (1, 7, 11, 65, 82, 83, 86, 273).

Ventricular hemodynamics. Animals are anesthetized and the right carotid artery cannulated with a microtip pressure transducer catheter (Millar SPR-240). The catheter is advanced into the left ventricle for the evaluation of the ventricular pressures and + and − dP/dt. A four-channel 100 kHz 16-bit recorder with built-in isolated ECG amplifier (iWorks IX-214) is used to store signals in a computer utilizing LabScribe software. The heart is then arrested in diastole with CdCl₂ and the myocardium fixed by perfusion with formalin. The left ventricular chamber is fixed at a pressure equal to the in vivo measured LVEDP (1, 7, 11, 65, 82, 83, 86, 273).

Integration of human myocardium with rat myocardium. Calcium transient in human myocytes (EGFP-positive) and non-human myocytes is determined by an ex vivo preparation and two-photon microscopy (1, 89). For cell physiology see Example 1 and refs. 1 and 89.

Coronary blood flow. This parameter is obtained with non-radioactive microspheres (see ref. 274).

PCR for Y-chromosome DNA: Primers are employed to detect Sry, the sex determining region of the Y-chromosome: humanSry-F: 5′-GAG AAG CTC TTC CTT CCT TTG CAC TG-3′ (26 nt, Tm 60° C.) and humanSry-R: 5′-TTC GGG TAT TTC TCT CTG TGC ATG GC-3′ (26 nt, Tm 61° C.) [amplicon size: 291 bp].

Detection of EGFP and human genes. For real-time RT-PCR, the infarcted myocardium is obtained from rat hearts treated with human PCs. RNA is extracted and reverse transcribed into cDNA. Specific primers are designed for the detection of EGFP, human Nkx2.5, MEF2C, SRF, GATA6, eNOS and E-cadherin.

Immunocytochemistry of myocardial regeneration. This includes analysis of proteins associated with cellular differentiation and electrical and mechanical coupling together with EGFP and Alu (see refs 1, 89).

Apoptosis-cell replication. Cell death is measured by TdT assay, hairpin 1 and hairpin 2 (1, 11, 275). Cycling cells are measured by Ki67, MCM5 and phospho-H3 for the detection of cells in the various phases of the cell cycle. The accumulation of newly formed cells with time is obtained on the basis of BrdU labeling.

Cell fusion and paracrine effects. For cell fusion see Example 1. Paracrine effects are determined on the basis of BrdU labeling in the surviving myocardium. This approach permits the quantitative assessment of the extent of regeneration in the non-EGFP non-Alu-positive myocytes and coronary vessels (1, 86, 89). Alternatively the injected cells could attenuate cell death or operate positively on the infarcted heart by both mechanisms. Thus, apoptotic and necrotic cell death in EGFP-negative cells is measured.

Having thus described in detail preferred embodiments of the present invention, it is to be understood that the invention defined by the appended claims is not to be limited by particular details set forth in the above description as many apparent variations thereof are possible without departing from the spirit or scope thereof.

Those skilled in the art will recognize, or be able to ascertain using no more than routine experimentation, many equivalents to the specific embodiments of the invention described herein. Such equivalents are intended to be encompassed by the following claims.

### REFERENCES

1. Bearzi C, Rota M, Hosoda T, Tillmanns J, Nascimbene A, De Angelis A, Yasuzawa-Amano S, Trofimova I, Siggins R W, Cascapera S, Beltrami A P, Zias E, Quaini F, Urbanek K, Michler R E, Bolli R, Kajstura J, Leri A, Anversa P: Human cardiac stem cells. Proc Natl Acad Sci USA. 104:14068-14073, 2007.
2. Messina E, De Angelis L, Frati G, Morrone S, Chimenti S, Fiordaliso F, Salio M, Battaglia M, Latronico M V G, Coletta M. Vivarelli E, Frati L, Cossu G, Giacomello A: Isolation and expansion of adult cardiac stem cells from human and murine heart. Circ Res. 95:911-921, 2004.
3. Smith R R, Barile L, Cho H C, Leppo M K, Hare J M, Messina E, Giacomello A, Abraham H R, Marban E: Regenerative potential of cardiosphere-derived cells expanded from percutaneous endomyocardial biopsy specimens. Circulation. 115:896-908, 2007.
4. Anversa P, Kajstura J, Leri A: If I can stop one heart from breaking. Circulation. 115:829-832, 2007.
5. Kawada H, Fujita J, Kingo K, Matsuzaki Y, Tsuma M, Miyatake H, Muguruma Y, Tsuboi K, Itabashi Y, Ikeda Y, Ogawa S, Okano H, Hotta T, Ando K, Fukuda K: Nonhematopoietic mesenchymal stem cells can be mobilized and differentiate into cardiomyocytes after myocardial infarction. Blood. 104:3581-3587, 2004.
6. Linke A, Muller P, Nurzynska D, Casarsa C, Torella D, Nascimbene A, Castaldo C, Cascapera S, Bohm M, Quaini F, Urbanek K, Leri A, Hintze T H, Kajstura J, Anversa P: Stem cells in the dog heart are self-renewing, clonogenic, and multipotent and regenerate infarcted myocardium, improving cardiac function. Proc Natl Acad Sci USA. 102:8966-8971, 2005.
7. Urbanek K, Rota M, Cascapera S, Bearzi C, Nascimbene A, De Angelis A, Hosoda T, Chimenti S, Baker M, Limana F, Nurzynska D, Torella D, Rotatori F, Rastaldo R, Musso E, Quaini F, Leri A, Kajstura J, Anversa P: Cardiac stem cells possess growth factor-receptor systems that after activation regenerate the infarcted myocardium, improving ventricular function and long-term survival. Circ Res. 97:663-673, 2005.
8. Harada M, Qin Y, Takano H, Minamino T, Zou Y, Toko H, Ohtsuka M, Matsuura K, Sano M, Nishi J, Iwanaga K, Akazawa H, Kunieda T, Zhu W, Hasegawa H, Kunisada K, Nagai T, Nakaya H, Yamauchi-Takihara K, Komuro I: G-CSF prevents cardiac remodeling after myocardial infarction by activating the Jak-Stat pathway in cardiomyocytes. Nat Med. 11:305-311, 2005.
9. Hasegawa H, Takano H, Iwanaga K, Ohtsuka M, Qin Y, Niitsuma Y, Ueda K, Toyoda T, Tadokoro H, Komuro I: Cardioprotective effects of granulocyte colony-stimulating factor in swine with chronic myocardial ischemia. J Am Coll Cardiol. 47:842-849, 2006.
10. Fukuda K, Fujita J, Hakuno D, Makino S: Mesenchymal stem-cell-derived cardiomyogenic cells in Cardiovascular regeneration and stem cell therapy. Edited by Leri A, Anversa P, Frishman W in Cardiovascular regeneration and stem cell therapy. Blackwell Futura, Malden Mass. pp. 37-46, 2007.
11. Beltrami A P, Barlucchi L, Torella D, Baker M, Limana F, Chimenti S, Kasahara H, Rota M, Musso E, Urbanek K, Leri A, Kajstura J, Nadal-Ginard B, Anversa P: Adult cardiac stem cells are multipotent and support myocardial regeneration. Cell. 114:763-776, 2003.
12. Oh H, Bradfute S B, Gallardo T D, Nakamura T, Gaussin V, Mishina Y, Pocius J, Michael L H, Behringer R R, Garry D J, Schneider M D: Cardiac progenitor cells from adult myocardium: homing, differentiation, and fusion after infarction. Proc Natl Acad Sci USA. 100:12313-12318, 2003.
13. Pfister O, Mouquet F, Jain M, Summer R, Helmes M, Fine A, Colucci W S, Liao R: CD31− but not CD31+ cardiac side populations cells exhibit functional cardiomyogenic differentiation. Circ Res. 97:52-61, 2005.
14. Tomita Y, Matsumura K, Wakamatsu Y, Matsuzaki Y, Shibuya 1, Kawaguchi H, Ieda M, Kanakubo S, Shinmazaki T, Ogawa S, Osumi N, Okano H, Fukuda K: Cardiac neural crest cells contribute to the dormant multipotent stem cell in the mammalian heart. J Cell Biol. 170:1135-1146, 2005.
15. Oyama T, Nagai T, Wada H, Naito A T, Matsuura K, Iwanaga K, Takahashi T, Goto M, Mikami Y, Yasuda N, Akazawa H, Uezumi A, Takeda S, Komuro I: Cardiac side population cells have a potential to migrate and differentiate into cardiomyocytes in vitro and in vivo. J Cell Biol. 176:329-341, 2007.
16. Shalaby F, Ho J, Stanford W L, Fischer K D, Schuh A C, Schwartz L, Bernstein A, Rossant J: A requirement for Flk1 in primitive and definitive hematopoiesis and vasculogenesis. Cell. 89:981-990, 1997.
17. Asahara T, Murohara T, Sullivan A, Silver M, van der Zee R, Li T, Witzenbichler B, Schatteman G, Isner J M: Isolation of putative progenitor endothelial cells for angiogenesis. Science. 275:964-967, 1997.
18. Yamashita J, Itoh H, Hirashima M, Ogawa M, Nishikawa S, Yurugi T, Naito M, Nakao K, Nishikawa S: Flk1-positive cells derived from embryonic stem cells serve as vascular progenitors. Nature. 408:92-96, 2000.
19. Carmeliet P: Mechanisms of angiogenesis and arteriogenesis. Nat Med. 6:389-395, 2000.
20. Aicher A, Heeschen C, Mildner-Rihm C, Urbich C, Ihling C, Technau-Ihling K, Zeiher A M, Dimmeler S: Essential role of endothelial nitric oxide synthase for mobilization of stem and progenitor cells. Nat Med. 9:1370-1376, 2003.
21. Urbich C, Dimmeler S: Endothelial progenitor cells: characterization and role in vascular biology. Circ Res. 95:343-353, 2004.
22. Asahara T, Kawamoto A: Endothelial progenitor cells for postnatal vasculogenesis. Am J Physiol. 287:C572-579, 2004.
23. Cossu G, Bianco P: Mesoangioblasts—vascular progenitors for extravascular mesodermal tissues. Curr Opin Genet Dev. 13:537-542, 2003.
24. Matsumoto K, Yoshitomi H, Rossant J, Zaret K S. Liver organogenesis promoted by endothelial cells prior to vascular function. Science. 294:559-563, 2001.
25. Kouskoff V, Lacaud G, Schwantz S, Fehling H J and Keller G: Sequential development of hematopoietic and cardiac mesoderm during embryonic stem cell differentiation. Proc Natl Acad Sci USA. 102:13170-13175, 2005.
26. Kattman S J, Huber T L, Keller G M: Multipotent flk-1+ cardiovascular progenitor cells give rise to the cardiomyocyte, endothelial, and vascular smooth muscle lineages. Dev Cell. 11:723-732, 2006.
27. Carmeliet P: Angiogenesis in life, disease and medicine. Nature. 438:932-936, 2005.
28. Coultas L, Chawengsaksophak K, Rossant J: Endothelial cells and VEGF in vascular development. Nature. 438:937-945, 2005.
29. Shi S, Gronthos S: Perivascular niche of postnatal mesenchymal stem cells in human bone marrow and dental pulp. J Bone Miner Res. 18:696-704, 2003.
30. Doherty M J, Ashton B A, Walsh S, Beresford J N, Grant M E, Canfield A E: Vascular pericytes express osteogenic potential in vitro and in vivo. J Bone Miner Res. 13:828-838, 1998.
31. Doherty M J, Canfield A E: Gene expression during vascular pericyte differentiation. Crit Rev Eukaryot Gene Expr. 9:1-17, 1999.
32. Shalaby F, Rossant Y, Yamagucci T P, Gertsenstein M, Wu X F, Breitman M L, Schuh A C: Failure of blood-island formation and vasculogenesis in Flk-1 deficient mice. Nature. 376:62-66, 1995.
33. Ema M, Faloon P, Zhang W J, Hirashima M, Reid T, Stanford W L, Orkin S, Choi K, Rossant J: Combinatorial effects of Flk1 on vascular and hematopoietic development in the mouse. Genes Dev. 17:380-393, 2003.
34. Reese D E, Mikawa T, Bader D M: Development of the coronary vessel system. Circ Res. 91:761-768, 2002.
35. Wada A M, Willet S G, Bader D: Coronary vessel development: a unique form of vasculogenesis. Arterioscler Thromb Vasc Biol. 23:2138-2145, 2003.
36. Huber T L, Kouskoff V, Fehling H J, Palis J, Keller G: Haemangioblast commitment is initiated in the primitive streak of the mouse embryo. Nature. 432:625-630, 2004.
37. Ema M, Takahashi S, Rossant J: Deletion of the selection cassette, but not cis-acting elements, in targeted Flk1-lacZ allele reveals Flk1 expression in multipotent mesodermal progenitors. Blood. 107:111-117, 2006.
38. Wu S M, Fujiwara Y, Cibulsky S M, Clapham D E, Lien C, Schultheiss T M, Orkin S H: Developmental origin of a bipotential myocardial and smooth muscle cell precursor in the mammalian heart. Cell. 127:1-14, 2006.
39. Kondo M, Wagers A J, Manz M G, Prohaska S S, Scherer D C, Beilhack G F, Shizuru J A, Weissman I L: Biology of hematopoietic stem cells and progenitors: implications for clinical application. Annu Rev Immunol. 21:759-806, 2003.
40. Miliarias D, Karasavvidou F, Papanikolaou A, Sioutopoulou D: KIT expression in fetal, normal adult, and neoplastic renal tissues. J Clin Pathol. 57:463-466, 2004.
41. Dan Y Y, Riehle K J, Lazaro C, Teoh N, Haque J, Campbell J S, Fausto N: Isolation of multipotent progenitor cells from human fetal liver capable of differentiating into liver and mesenchymal lineages. Proc Natl Acad Sci USA. 103:9912-9917, 2006.
42. Min K W, Leabu M: Interstitial cells of Cajal (ICC) and gastrointestinal stromal tumor (GIST): facts, speculations, and myths. J Cell Mol Med. 10:995-1013, 2006.
43. Miettinen M, Lasota J: KIT (CD1117): a review on expression in normal and neoplastic tissues, and mutations and their clinicopathologic correlation. Appl Immunohistochem Mol Morphol. 13:205-220, 2005.
44. Wehrle-Haller B, Weston J A: Altered cell-surface targeting of stem cell factor causes loss of melanocyte precursors in Steel 17H mutant mice. Dev Biol. 210:71-86, 1999.
45. Teyssier-Le Discorde M, Prost S, Nandrot E, Kirszenbaum M: Spatial and temporal mapping of c-kit and its ligand, stem cell factor expression during human embryonic haemopoiesis. Br J Haematol. 107:247-253, 1999.
46. Beauvais-Jouneau A, Pla P, Bernex F, Dufour S, Salamero J, Fassler R, Panthier J J, Thiery J P, Larue L: A novel model to study the dorsolateral migration of melanoblasts. Mech Dev. 89:3-14, 1999.
47. Yoshida H, Kunisada T, Grimm T, Nishimura E K, Nishioka E, Nishikawa S I: Review: melanocyte migration and survival controlled by SCF/c-kit expression. J Investig Dermatol Symp Proc. 6:1-5, 2001.
48. Peters E M, Tobin D J, Botchkareva N, Maurer M, Paus R: Migration of melanoblasts into the developing murine hair follicle is accompanied by transient c-kit expression. J Histochem Cytochem. 50:751-766, 2002.
49. Erlandsson A, Larsson J, Forsberg-Nilsson K: Stem cell factor is a chemoattractant and a survival factor for CNS stem cells. Exp Cell Res. 301:201-210, 2004.
50. De Felici M: Regulation of primordial germ cell development in the mouse. Int J Dev Biol. 44:575-580, 2000.
51. Shimizu M, Minakuchi K, Tsuda A, Hiroi T, Tanaka N, Koga J, Kiyono H: Role of stem cell factor and c-kit signaling in regulation of fetal intestinal epithelial cell adhesion to fibronectin. Exp Cell Res. 266:311-322, 2001.
52. Nishikawa M, Tahara T, Hinohara A, Miyajima A, Nakahata T, Shimosaka A: Role of the microenvironment of the embryonic aorta-gonad-mesonephros region in hematopoiesis. Ann NY Acad. Sci. 938:109-116, 2001.
53. Nobuhisa I, Takizawa M, Takaki S, Inoue H, Okita K, Ueno M, Takatsu K, Taga T: Regulation of hematopoietic development in the aorta-gonad-mesonephros region mediated by Lnk adaptor protein. Mol Cell Biol. 23:8486-8494, 2003.
54. Matsui Y, Zsebo K M, Hogan B L: Embryonic expression of a haematopoietic growth factor encoded by the S1 locus and the ligand for c-kit. Nature. 347:667-669, 1990.
55. Simmons P J, Aylett G W, Niutta S, To L B, Juttner C A, Ashman L K: C-kit is expressed by primitive human hematopoietic cells that give rise to colony-forming cells in stroma-dependent or cytokine-supplemented culture. Exp Hematol. 22:157-165, 1994.
56. Yokota T, Huang J, Tavian M, Nagai Y, Hirose J, Zuniga-Pflucker J C, Peault B, Kincade P W: Tracing the first waves of lymphopoiesis in mice. Development. 133:2041-2051, 2006.
57. Kunisada T, Yoshida H, Yaazaki H, Miyamoto A, Hemmi H, Nishimura E, Shultz L D, Nishikawa S-I, Hayashi S-I: Transgene expression of steel factor in the basal layer of epidermis promotes survival, proliferation, differentiation and migration of melanocyte precursors. Development. 125:2915-2923, 1998.
58. Urbanek K, Quaini F, Tasca G, Torella D, Castaldo C, Nadal-Ginard B, Leri A, Kajstura J, Quaini E, Anversa P: Intense myocyte formation from cardiac stem cells in human cardiac hypertrophy. Proc Natl Acad Sci USA. 100:10440-10445, 2003.
59. Urbanek K, Torella D, Sheikh F, De Angelis A, Nurzynska D, Silvestri F, Beltrami C A, Bussani R, Beltrami A P, Quaini F, Bolli R, Leri A, Kajstura J, Anversa P: Myocardial regeneration by activation of multipotent cardiac stem cells in ischemic heart failure. Proc Natl Acad Sci USA. 102:8692-8697, 2005.
60. Geissler E N, Ryan M A, Housman D E: The dominant-white spotting (W) locus of the mouse encodes the c-kit proto-oncogene. Cell. 55:185-192, 1988.
61. Nocka K, Majumder S, Chabot B, Ray P, Cervone M, Bernstein A, Besmer P: Expression of c-kit gene products in known cellular targets of W mutations in normal and W mutant mice—evidence for an impaired c-kit kinase in mutant mice. Genes Dev. 3:816-826, 1989.
62. Reith A D, Rottapel R, Giddens E, Brady C, Forrester L, Bernstein A: W mutant mice with mild or severe developmental defects contain distinct point mutations in the kinase domain of the c-kit receptor. Genes Dev. 4:390-400, 1990.
63. Alexander W S, Lyman S D, Wagner E F: Expression of functional c-kit receptors rescues the genetic defect of W mutant mast cells. EMBO J. 10:3683-3691, 1991.
64. Migliaccio A R, Carta C, Migliaccio G: In vivo expansion of purified hematopoietic stem cells transplanted in non-ablated W/Wv mice. Exp Hematol. 27:1655-1666, 1999.
65. Dawn B, Stein A B, Urbanek K, Rota M, Whang B, Rastaldo R, Torella D, Tang X L, Rezazadeh A, Kajstura J, Leri A, Hunt G, Varma J, Prabhu S D, Anversa P, Bolli R: Cardiac stem cells delivered intravascularly traverse the vessel barrier, regenerate infracted myocardium, and improve cardiac function. Proc Natl Acad Sci USA. 102:3766-3771, 2005.
66. Bearzi C, Muller P, Amano K, Tang X-L, Loredo M, Mosna F, Gatti A, Esposito G, Leri A, Kajstura J, Anversa P, Rimoldi O, Bolli R: Identification and characterization of cardiac stem cells in the pig heart. Circulation. 114:II-125, 2006.
67. Bolli R, Jneid H, Tang X-L, Rimoldi O, Mosna F, Loredo M, Gatti A, Kajstura J, Leri A, Bearzi C, Abdel-Latif A, Anversa P: Intracoronary administration of cardiac stem cells improves cardiac function in pigs with old infarction. Circulation. 114:II-239, 2006.
68. Leri A, Kajstura J, Anversa P: Cardiac stem cells and mechanisms of myocardial regeneration. Physiol Rev. 85:1373-1416, 2005.
69. Anversa P, Kajstura J, Leri A, Bolli R: Life and death of cardiac stem cells: a paradigm shift in cardiac biology. Circulation. 113:1451-1463, 2006.
70. Anversa P, Leri A, Rota M, Hosoda T, Bearzi C, Urbanek K, Kajstura J, Bolli R: Concise review: stem cells, myocardial regeneration, and methodological artifacts. Stem Cells. 25:589-601, 2007.
71. Wagers A J, Sherwood R I, Christensen J L, Weissman I L: Little evidence for developmental plasticity of adult hemopoietic stem cells. Science. 297:2256-2259, 2002.
72. Wagers A J, Weissman I L: Plasticity of adult stem cells. Cell. 116:639-648, 2004.
73. Balsam L B, Wagers A J, Christensen J L, Kofidis T, Weissman I L, Robbins R C: Haematopoietic fates in ischaeaemic myocardium. Nature. 428:668-673, 2004.
74. Fazel S, Cimini M, Chen L, Li S, Angoulvant D, Fedak P, Verma S, Weisel R D, Keating A, Li R K: Cardioprotective c-kit+ cells are from the bone marrow and regulate the myocardial balance of angiogenic cytokines. J Clin Invest. 116:1865-1877, 2006.
75. Quaini F, Urbanek K, Beltrami A P, Finato N, Beltrami C A, Nadal-Ginard B, Kajstura J, Leri A, Anversa P: Chimerism of the transplanted heart. N Engl J Med. 343:5-15, 2002.
76. Schwartz R S, Curfman G D: Can the heart repair itself? N Engl J Med. 346:2-4, 2002.
77. Bolli R: Regeneration of the human heart: no chimera? N Engl J Med. 346:55-56, 2002.
78. Thiele J, Varus E, Wickenhauser C, Kvasnicka H M, Lorenzen J, Gramley F, Metz K A, Riviero F, Beelan D W: Mixed chimerism of cardiomyocytes and vessels after allogenic bone marrow and stem-cell transplantation in comparison with cardiac allografts. Transplantation. 77:1902-1905, 2004.
79. Jackson K A, Majka S M, Wang H, Pocius J, Hartley C J, Majesky M W, EntmanM L, Michael L H, Hirschi K K, Goodell M A: Regeneration of ischemic cardiac muscle and vascular endothelium by adult stem cells. J Clin Invest. 107:1395-1402, 2001.
80. Laflamme M A, Murray C E: Regenerating the heart. Nat. Biotechnol. 23:845-856, 2005.
81. Rubart M, Field L J: Cardiac regeneration: repopulating the heart. Annu Rev Physiol. 68:29-49, 2006.
82. Orlic D, Kajstura J, Chimenti S, Jakoniuk I, Anderson S M, Li B, Pickel J, McKay R, Nadal-Ginard B, Bodine D M, Leri A, Anversa P: Bone marrow cells regenerate infarcted myocardium. Nature. 410:701-705, 2001.
83. Orlic D, Kajstura J, Chimenti S, Limana F, Jakoniuk I, Quaini F, Nadal-Ginard B, Bodine D M, Leri A, Anversa P: Mobilized bone marrow cells repair the infarcted heart improving function and survival. Proc Natl Acad Sci USA. 98:10344-10349, 2001.
84. Bradorff C, Brandes R P, Popp R, Rupp S, Urbich C, Aicher A, Fleming I, Busse R, Zeiher A M, Dimmeler S: Transdifferentiation of blood-derived human adult endothelial progenitor cells into functional active cardiomyocytes. Circulation. 107:1024-1032, 2003.
85. Koyanagi M, Brandes R P, Haendeler J, Zeiher A M, Dimmeler S: Cell-to-cell connection of endothelial progenitor cells with cardiac myocytes by nanotubes: a novel mechanism for cell fate changes? Circ Res. 96:1039-1041, 2005.
86. Kajstura J, Rota M, Whang B, Cascapera S, Hosoda T, Bearzi C, Nurzynska D, Kasahara H, Zias E, Bonafe' M, Nadal-Ginard B, Torella D, Nascimbene A, Quaini F, Urbanek K, Leri A, Anversa P: Bone marrow cells differentiate in cardiac cell lineages after infarction independently of cell fusion. Circ Res. 96:127-137, 2005.
87. Murasawa S, Kawamoto A, Horii M, Nakamori S, Asahara T: Niche-dependent translineage commitment of endothelial progenitor cells, not cell fusion in general, into myocardial lineage cells. Arterioscler Thromb Vasc Biol. 25:1388-1394, 2005.
88. Yoon Y S, Wecker A, Heyd L, Park J S, Tkebuchava T, Kusano K, Hanley A, Scadova H, Qin G, Cha D H, Johnson K L, Aikawa R, Asahara T, Losordo D W: Clonally expanded novel multipotent stem cells from human bone marrow regenerate myocardium after myocardial infarction. J Clin Invest. 115:326-338, 2005.
89. Rota M, Kajstura J, Hosoda T, Bearzi C, Vitale S, Esposito G, Iaffaldano G, Padin-Ireguas M E, Gonzalez A, Rizzi R, Small N, Muraski J, Alvarez R, Chen X, Urbanek K, Bolli R, Houser S R, Leri A, Sussman M A, Anversa P: Bone marrow cells adopt the cardiomyogenic fate in vivo. Proc Natl Acad Sci USA. In press, 2007.
90. Tosh D, Slack J M: How cells change their phenotype. Nat Rev Mol Cell Biol. 3:187-194, 2002.
91. Pomerantz J, Blau H M: Nuclear reprogramming: a key to stem cell function in regenerative medicine. Nat Cell Biol. 6:810-816, 2004.
92. Leri A, Kajstura J, Anversa P: Identity deception: not a crime for a stem cell. Physiology. 20:162-168, 2005.
93. Lockhart D J, Dong H, Byrne M C, Follettie M T, Gallo M V, Chee M S, Mittmann M, Wang C, Kobayashi M, Horton H, Brown E L: Expression monitoring by hybridization to high-density oligonucleotide arrays. Nat Biotechnol. 14:1675-1680, 1996.
94. Li C, Wong W H: Model-based analysis of oligonucleotide arrays: expression index computation and outlier detection. Proc Natl Acad Sci USA. 98:31-36, 2001.
95. Bhattacharya B, Miura T, Brandenberger R, Mejido J, Luo Y, Yang A X, Joshi B H, Ginis I, Thies R S, Amit M, Lyons I, Condie B G, Itskovitz-Eldor J, Rao M S, Puri R K: Gene expression in human embryonic stem cell lines: unique molecular signature. Blood. 103:956-2964, 2004.
96. Bruno L, Hoffmann R, McBlane F, Brown J, Gupta R, Joshi C, Pearson S, Seidl T, Heyworth C, Enver T: Molecular signatures of self-renewal, differentiation, and lineage choice in multipotential hemopoietic progenitor cells in vitro. Mol Cell Biol. 24: 741-756, 2004.
97. Ivanova N B, Dimos J T, Schaniel C, Hackney J A, Moore K A, Lemischka I R: A stem cell molecular signature. Science. 298:601-604, 2002.
98. Ramalho-Santos M S, Yoon Y, Matsuzxaki R, Mulligan C, Melton D A: “Stemness” transcriptional profiling of embryonic and adult stem cells. Science. 298:597-600, 2002.
99. Sturn A, Quackenbush J, Trajanoski Z: Genesis: cluster analysis of microarray data. Bioinformatics. 18:207-208, 2002.
100. Moore K A: Recent advances in defining the hemopoietic stem cell niche. Curr Opin Hematol. 11:107-111, 2004.
101. Ivanova N B, Dimos J T, Schaniel C, Hackney J A, Moore K A, Lemischka I R: A stem cell molecular signature. Science. 298:601-604, 2002.
102. Ramalho-Santos M S, Yoon Y, Matsuzaki R, Mulligan C, Melton D A: ‘Stemness’ transcriptional profiling of embryonic and adult stem cells. Science. 298:597-600, 2002.
103. Bruno L, Hoffmann R, McBlane F, Brown J, Gupta R, Joshi C, Pearson S, Seidl T, Heyworth C, Enver T: Molecular signatures of self-renewal, differentiation, and lineage choice in multipotential hemopoietic progenitor cells in vitro. Mol Cell Biol. 24:741-756, 2004.
104. Bhattacharya B, Cai J, Luo Y, Miura T, Mejido J, Brimble S N, Zeng X, Schultz T C, Rao M S, Puri P K: Comparison of the gene expression profile of undifferentiating embryoid bodies. BMC Dev Biol. 5:22, 2005.
105. Player A, Wang Y, Bhattacharya B, Rao M, Puri R K, Kawasaki E S: Comparisons between transcriptional regulation and RNA expression in human embryonic stem cell lines. Stem Cells Dev. 15:315-323, 2006
106. Mikkelsen T S, Ku M, Jaffe D B, Isaac B, Lieberman E, Giannoukos G, Alvarez P, Brockman W, Kim T K, Koche R P, Lee W, Mendenhall E, O'Donovan A, Presser A, Russ C, Xie X, Meissner A, Wernig M, Jaenisch C, Lander E, Bernstein B: Genome-wide maps of chromatin state in pluripotent and lineage-committed cells. Nature. 448:553-560, 2007.
107. Gan Q, Yoshida T, McDonald O, Owens G: Epigenetic mechanisms contribute to pluripotency and cell linkage determination of embryonic stem cells. Stem Cells. 25:2-9, 2007.
108. Spivakov M, Fischer A G: Epigenetic signatures of stem-cell identity. Nat Rev Genet. 8:263-271, 2007.
109. Reik W: Stability and flexibility of epigenetic gene regulation in mammalian development. Nature. 447:425-432, 2007.
110. Jenuwein T, Allis C D: Translating the histone code. Science. 293:1074-1080, 2001.
111. Mellor J: Dynamic nucleosomes and gene transcription. Trends Genet. 22:320-329, 2006.
112. Boyer L A, Mathur D, Jaenisch R: Molecular control of pluripotency. Curr Opin Genet Dev. 16:455-462, 2006.
113. Azuara V, Perry P, Sauer S, Spivakov M, Jorgensen H F, John R M, Gouti M, Casanova M, Warnes G, Merkenschlager M, Fisher A G: Chromatin signatures of pluripotent cell lines. Nat Cell Biol. 8:532-538, 2006
114. Klose R J, Kallin E M, Zhang Y: JmjC-domain-containing proteins and histone demethylation. Nat Rev Genet. 7:715-727, 2006.
115. Hattori N, Nishino K, Ko Y G, Hattori N, Ohgane J, Tanaka S, Shiota K: Epigenetic control of mouse Oct-4 gene expression in embryonic stem cells and trophoblast stem cells. J Biol Chem. 279:17063-17069, 2004.
116. Kimura H, Tada M, Nakatsuji N, Tada T: Histone code modifications on pluripotential nuclei of reprogrammed somatic cells. Mol Cell Biol. 24:5710-5720, 2004.
117. O'Neill L P, VerMilyea M D, Turner B M: Epigenetic characterization of the early embryo with a chromatin immunoprecipitation protocol applicable to small cell populations. Nat Genet. 38:835-841, 2006.
118. Li E: Chromatin modification and epigenetic reprogramming in mammalian development. Nat Rev Genet. 3:662-673, 2002.
119. Szutorisz H, Canzonetta C, Georgiou A, Chow C M, Tora L, Dillon N: Formation of an active tissue-specific chromatin domain initiated by epigenetic marking at the embryonic stem cell stage. Mol Cell Biol. 25:1804-1820, 2005.
120. Meshorer E, Yellajoshula D, George E, Scambler P J, Brown D T, Mistell T: Hyperdynamic plasticity of chromatin proteins in pluripotent embryonic stem cells. Dev Cell. 10:105-116, 2006.
121. Buszczak M, Spradling A C: Searching chromatin for stem cell identity. Cell. 125:233-236, 2006.
122. Miller F D, Gauthier A S: Timing is everything: making neurons versus glia in the developing cortex. Neuron. 54:357-369, 2007.
123. Anversa P, Leri A, Kajstura J: Cardiac regeneration. J Am Coll Cardiol. 47:1769-1776, 2006.
124. Jaenish R, Bird A: Epigenetic regulation of gene expression: how the genome integrates intrinsic and environmental signals. Nat Genet. 33:245-254, 2003.
125. Bird A: DNA methylation patterns and epigenetic memory. Genes Dev. 16: 6-21, 2002.
126. Boyes J, Bird A: Repression of genes by DNA methylation depends on CpG density and promoter strength: evidence for involvement of a methyl-CpG binding protein. EMBO J. 11:327-333, 1992.
127. Hsieh C L: Dependence of transcriptional repression on CpG methylation density. Mol Cell Biol. 14:5487-5494, 1994.
128. Brunk B P, Goldhamer D J, Emerson C P Jr: Regulated demethylation of the myoD distal enhancer during skeletal myogenesis. Dev Biol. 177:490-503, 1996.
129. Palacios D, Puri P L: The epigenetic network regulating muscle development and regeneration. J Cell Physiol. 207:1-11, 2006.
130. Chan Y, Fish J E, D'Abreo C, Lin S, Robb G B, Teichert A M, Karantzoulis-Fegaras F, Keightley A, Steer B M, Marsden P A: The cell specific expression of endothelial nitric-oxide synthase: a role for DNA methylation. J Biol Chem. 279:35087-35100, 2004.
131. Esteller M: Cancer epigenomics: DNA methylomes and histone modification maps. Nat Rev Genet. 8:286-298, 2007.
132. Bernstein B E, Mikkelson T S, Xie X, Kamal M, Huebert D J, Cuff J, Fry B, Meissner A, Wernig M, Plath K, Jaenisch R, Wagschal A, Feil R, Schreiber S L, Lander E S: A bivalent chromatin structure marks key developmental genes in embryonic stem cells. Cell. 125:315-326, 2006.
133. Francis N J, Kingston R E, Woodcock C L: Chromatin compaction by a polycomb group protein complex. Science. 306:1574-1577, 2004.
134. Ringrose L, Paro R: Epigenetic regulation of cellular memory by the polycomb and Trithorax group proteins. Annu Rev Genet. 38:413-443, 2004.
135. Santos-Rosa H, Caldas C: Chromatin modifier enzymes, the histone code and cancer. Eur J Cancer. 41:2381-2402, 2005.
136. Pray-Grant M G, Daniel J A, Schietz D, Yates J R 3rd, Grant P A: Chd1 chromodomain links histone H3 methylation with SAGA- and SLIK-dependent acetylatin. Nature. 433:434-438, 2005.
137. Sims R J 3rd, Nishioka K, Reinberg D: Histone lysine methylation: a signature for chromatin function. Trends Genet. 19:629-639, 2003.
138. Wysocka J, Milne T A, Allis C D: Taking LSD to a new high. Cell. 122:654-658, 2005.
139. Schneider R, Bannister A J, Myers Fa, Thorne A W, Crane-Robinson C, Kouzarides T: Histone H3 lysine 4 methylation patterns in higher eukaryotic genes. Nat Cell Biol. 6:73-77, 2004.
140. Feldman N, Gerson A, Fang J, Li E, Zhang Y, Shinkai Y. Cedar H, Bergman Y: Gpa-mediated irreversible epigenetic inactivation of Oct-3/4 during early embryogenesis. Nat Cell Biol. 8:188-194, 2006.
141. Henikoff S, Furuyama T, Ahmad K: Histone variants, nucleosome assembly and epigenetic inheritance. Trends Genet. 20:320-326, 2004.
142. van Steensel B: Mapping of genetic and epigenetic regulatory networks using microarrays. Nat Genet. 37 Suppl:S18-24, 2005.
143. Bottardi S, Aumont A, Grosveld F, Milot E: Developmental stage-specific epigenetic control of human beta-globin gene expression is potentiated in hematopoietic progenitor cells prior to their transcriptional activation. Blood. 102:3989-3997, 2003.
144. Attema J L, Papathanasiou P, Forsberg E C, Xu J, Smale S T, Weissman I L: Epigenetic characterization of hematopoietic stem cell differentiation using miniChiP and bisulfate sequencing analysis. Proc Natl Acad Sci USA. 104:12371-12376, 2007.
145. Chambers S M, Shaw C A, Gatza C, Fisk C J, Donehower L A, Goodell M A: Aging hematopoietic stem cells decline in function and exhibit epigenetic dysregulation. PloS Biol. 5:e201, 2007.
146. Illi B, Scopece A, Nanni S, Farsetti A, Morgante L, Biglioli P, Capogrossi M C, Gaetano C: Epigenetic histone modification and cardiovascular lineage programming in mouse embryonic stem cells exposed to laminar shear stress. Circ Res. 96:501-508, 2005.
147. Hall I M, Shankaranarayana G D, Noma K, Ayoub N, Cohen A, Grewai S I: Establishment and maintenance of a heterochromatin domain. Science. 297:2232-2237, 2002.
148. Fischle W, Wang Y, Jacobs S A, Kim Y, Allis C D, Khorasanizadeh S: Molecular basis for the discrimination of regressive methyllysine marks in histone H3 by Polycomb and HP1 chromodomains. Genes Dev. 17:1870-1881, 2003.
149. Frontelo P, Leader J E, Yoo N, Potocki A C, Crawford M, Kulik M, Lechleider R J: Suv39h histone methyltransferases interact with Smads and cooperate in BMP-induced repression. Oncogene. 23:5242-5251, 2004.
150. Shi Y, Lan F, Matson C, Mulligan P, Whetstine J R, Cole P A, Casero R A, Shi Y: Histone demethylation mediated by the nuclear amine oxidase homolog LSD1. Cell. 119:941-953, 2004.
151. Tyteca S, Legube G, Trouche D: To die or not to die: a HAT trick. Mol Cell. 24:807-808, 2006.
152. Schuettengruber B, Chourrout D, Vervoort M, Leblanc B, Cavalli G: Genome regulation by polycomb and trithorax proteins. Cell. 128:735-745, 2007.
153. Lee K K, Workman J L: Histone acetyltransferase complexes: one size doesn't fit all. Nat Rev Mol Cell Biol. 8:284-295, 2007.
154. Yang X J, Seto E: HATs and HDACs: from structure, function and regulation to novel strategies for therapy and prevention. Oncogene. 26:5310-5318, 2007.
155. Chang S, Young B D, Li S, Oi X, Richardson J A, Olson E N: Histone deacetylase 7 maintains vascular integrity by repressing matrix metalloproteinase 10. Cell. 126:321-324, 2006.
156. Potente M, Ghaeni L, Baldessari D, Mostoslavsky R, Rossig L, Dedquiedt F, Haendeler J, Mione M, Dejana E, Alt F W, Zeiher A M, Dimmeler S: SIRT 1 controls endothelial functions during postnatal growth. Genes Dev. In press, 2007.
157. Schachinger V, Erbs S, Elsasser A, Haberbosch W, Hambrecht R, Holschermann H, Yu J, Corti R, Mathey D G, Hamm C W, Suselbeck T, Assmus B, Tonn T, Dimmeler S, Zeiher A M: Intracoronary bone marrow-derived progenitor cells in acute myocardial infarction. N Engl J Med. 355:1210-1221, 2006.
158. Assmus B, Honold J, Schachinger V, Britten M B, Fischer-Rasokat U, Lehmann R, Teupe C, Pistorius K, Martin H, Abolmaali N D, Tonn T, Dimmeler S, Zeiher A M: Transcoronary transplantation of progenitor cells after myocardial infarction. N Engl J Med. 355:1222-1232, 2006.
159. Zhang C L, McKinsey T A, Chang S, Antos C L, Hill J A, Olson E N: Class II histone deacetylases act as signal-responsive repressors of cardiac hypertrophy. Cell. 110:479-488, 2002.
160. Chang S, McKinsey T A, Zhang C L, Richardson J A, Hill J A, Olson E N: Histone deacetylases 5 and 9 govern responsiveness of the heart to a subset of stress signals and play redundant roles in heart development. Mol Cell Biol. 24:8467-8476, 2004.
161. Backs J, Olson E N: Control of cardiac growth by histone acetylation/deacetylation. Circ Res. 98:15-24, 2006.
162. Trivedi C M, Luo Y, Yin Z, Zhand M, Zhu W, Wang T, Floss T, Gopettlicher M, Noppinger P R, Wurst W, Ferrari V A, Abrams C S, Gruber P J, Epstein J A: Hdac2 regulates the cardiac hypertrophic response by modulating Gsk3 beta activity. Nat Med. 13:324-331, 2007.
163. Montgomery R L, Davis C A, Poptthoff M J, Haberland M, Fielitz J, Qi X, Hill J A, Richardson J A, Olson E N: Histone deacetylases 1 and 2 redundantly regulate cardiac morphogenesis growth, and contractility. Genes Dev. 21:1790-1802, 2007.
164. McKinsey T A, Olson E N: Dual roles of histone deacetylases in the control of cardiac growth. Novartis Found Symp. 259:132-141, 2004.
165. Kawamura T, Ono K, Morimoto T, Wada H, Hirai M, Hidaka K, Morisaki T, Heike T, Nakahata T, Kita T, Hasegawa K: Acetylation of GATA-4 is involved in the differentiation of embryonic stem cells into cardiac myocytes. J Biol Chem. 280:19682-19688, 2005.
166. Hernandez M, Shao Q, Yang X J, Luh S P, Kandouz M, Batist G, Laird D W, Alaoui-Jamali M A: A histone deacetylation-dependent mechanism for transcriptional repression of the gap junction gene cx43 in prostate cancer cells. Prostate. 66:1151-61, 2006.
167. Gregoire S, Xiao L, Nie J, Zhang X, Xu M, Li J, Wong J, Seto E, Yang X J: Histone deacetylase 3 interacts with and deacetylates myocyte enhancer factor 2. Mol Cell Biol. 27:1280-1295, 2007.
168. Glozak M A, Seto E: Histone deacetylases and cancer. Oncogene. 26:5420-5432, 2007.
169. Sengupta N, Seto E: Regulation of histone deacetylase activities. J Cell Biochem. 93:57-67, 2004.
170. Gregoire S, Xiao L, Nie J, Zhang X, Xu M, Li J, Wong J, Seto E, Yang X J: Histone deacetylase 3 interacts with and deacetylates myocyte enhancer factor 2. Mol Cell Biol. 27:1280-1295, 2007.
171. Buggy J J, Sideris M L, Mak P, Lorimer D D, McIntosh B, Clark J M. Cloning and characterization of a novel human histone deacetylase, HDAC8. Biochem J. 350:199-205, 2000.
172. Waltregny D, Glennison W, Tran S L, North B J, Verdin E, Colige A, Castronovo V: Histone deacetylase HDAC8 associates with smooth muscle cell contractility. FASEB J. 19:966-968, 2005.
173. Martin M, Kettmann R, Dequiedt F: Class II histone deacetylases: regulating the regulators. Oncogene. 26:5450-5467, 2007.
174. deRuijter A J, van Gennip A H, Caron H N, Kemp S, vanKuilenburg A B: Histone deacetylases (HDACs): characterization of the classical HDAC family. Biochem J. 370:737-749, 2003.
175. Bertos N R, Wang A H, Yang X J: Class II histone deacetylases: structure, function, and regulation. Biochem Cell Biol. 79:243-252, 2001.
176. Miska E A, Karisson C, Langley E, Nielson S J, Pines J, Kouzarides T: HDAC4 deacetylase associates with and represses the MEF2 transcription factor. EMBO J. 18:5099-5107, 1999.
177. Sparrow D B, Miska E A, Langley E, Reynaud-Deonauth S, Kotecha S, Towers N, Spohr G, Kouzarides T, Mohun T J: MEF-2 function is modified by a novel co-repressor, MITR. EMBO J. 18:5085-5098, 1999.
178. Wang A H, Bertos N R, Vezmar M, Pelletier N, Crosato M, Henh H H, Th'ng J, Han J, Yang X J: HDAC4, a human histone deacetylase related to yeast HDA1, is a transcriptional corepressor. Mol Cell Biol. 19:7816-27, 1999.
179. Lemercier C, Verdel A, Balloo B, Curtet S, Brocard M P, Khochbin S: mHDA1/HDAC5 histone deacetylase interacts with and represses MEF2A transcriptional activity. J Biol Chem. 275:15594-15599, 2000.
180. Lu J, McKinsey T A, Nicol R L, Olson E N: Signal-dependent activation of the MEF2 transcription factor by disassociation from histone deacetylases. Proc Natl Acad Sci USA. 97:4070-4075, 2000.
181. Potthoff M J, Wu H, Arnold M A, Shelton J M, Backs J, McAnally J, Richardson J A, Bassel-Duby R, Olson E N: Histone deacetylase degradation and MEF2 activation promote the formation of slow-twitch myofibers. J Clin Invest. 117:2459-2467, 2007.
182. Zhao X, Ito A, Kane C D, Liao T S, Bolger T A, Lemrow S M, Means A R, Yao T P: The modular nature of histone deacetylase HDAC4 confers phosphorylation-dependent intracellular trafficking. J Biol Chem. 276:35042-35048, 2001.
183. Grozinger C M, Schreiber S L: Regulation of histone deacetylase 4 and 5 and transcriptional activity by 14-3-3-dependent cellular localization. Proc Natl Acad Sci USA. 97:7835-7840, 2000.
184. McKinsey T A, Zhang C L, Lu J, Olson E N: Signal-dependent nuclear export of a histone deacetylase regulates muscle differentiation. Nature. 408:106-111, 2000.
185. Davis F J, Gupta M, Camoretti-Mercado B, Schwartz R J, Gupta M P: Calcium/calmodulin-dependent protein kinase activates serum response factor transcription activity by its disassociation from histone deacetylase, HDAC4. Implications in cardiac muscle gene regulation during hypertrophy. J Biol Chem. 278:20047-20058, 2003.
186. Yang X J, Gregoire S: Class II histone deacetylases: from sequence to function, regulation, and clinical implication. Mol Cell Biol. 25:2873-2884, 2005.
187. Kawaguchi Y, Kovacs J J, McLaurin A, Vance J M, Ito A, Yao T P: The deacetylase HDAC6 regulates aggresome formation and cell viability in response to misfolded protein stress. Cell. 115:727-738, 2003.
188. Michishita E, Park J Y, Burneskis J M, Barrett J C, Horikawa I: Evolutionarily conserved and nonconserved cellular localizations and functions of human SIRT proteins. Mol Biol Cell. 16:4623-4635, 2005.
189. Michan S, Sinclair D: Sirtuins in mammals: insights into their biological function. Biochem J. 404:1-13, 2007.
190. Frye R A: Phylogenetic classification of prokaryotic and eukaryotic Sir2-like proteins. Biochem Biophys Res Commun. 273:793-798, 2000.
191. Vaziri H, Dessain S K, Ng Eaton E, Imai S I, Frye R A, Pandita T K, Guarente L, Weinberg R A: hSIR2(SIRT1) functions as a NAD-dependent p53 deacetylase. Cell. 107:149-158, 2001.
192. North B J, Marshall B L, Borra M T, Denu J M, Verdin E: The human Sir2 ortholog, SIRT2, is an NAD+-dependent tubulin deacetylase. Mol Cell. 11:437-444, 2003.
193. Shi T, Wang F, Stieren E, Tong Q: SIRT3, a mitrochondrial sirtuin deacetylase, regulates mitochondrial function and thermogenesis in brown adipocytes. J Biol Chem. 280:13560-13567, 2005.
194. Imai S, Armstrong C M, Kaeberlein M, Guarente L: Transcriptional silencing and longevity protein Sir2 is an NAD-dependent histone deacetylase. Nature. 403:795-800, 2000.
195. Vaquero A, Scher M B, Lee D H, Sutton A, Cheng H L, Alt F W, Serrano L, Sternglanz R, Reinberg D: SirT2 is a histone deacetylase with preference for histone H4 Lys 16 during mitosis. Genes Dev. 20:1256-1261, 2006.
196. Bouras T, Fu M, Sauve A A, Wang F, Quong A A, Perkins N D, Hay R T, Gu W, Pestell R G: SIRT1 deacetylation and repression of p300 involves lysine residues 1020/1024 within the cell cycle regulatory domain 1. J Biol Chem. 280:10264-10276, 2005.
197. Luo J, Nikolaev A Y, Imai S, Chen D, Su F, Shiloh A, Guarente L, Gu W: Negative control of p53 by Sir2alpha promotes cell survival under stress. Cell. 107:137-148, 2001.
198. Gregoire S, Xiao L, Nie J, Zhang X, Xu M, Li J, Wong J, Seto E, Yang X J: Histone deacetylase 3 interacts with and deacetylates myocyte enhancer factor 2. Mol Cell Biol. 27:1280-1295, 2007.
199. Gao L, Cueto M A, Asselbergs F, Atadja P: Cloning and functional characterization of HDAC11, a novel member of the human histone deacetylase family. J Biol Chem. 277:25748-25755, 2002.
200. Knowlton R G, Brown V A, Braman J C, Barker D, Schumm J W, Murray C, Takvorian T, Ritz J, Donis-Keller H: Use of highly polymorphic DNA probes for genotypic analysis following bone marrow transplantation. Blood. 68:378-385, 1986.
201. Yam P, Petz L D, Ali S, Stock A D, Wallace R B: Development of a single probe for documentation of chimerism following bone marrow transplantation. Am J Hum Genet. 41:867-881, 1987.
202. Martinelli G, Trabetti E, Farabegoli P, Testoni N, Bandini G, Motta M R, Vittone A, Terragna C, Pignatti P F, Tura S: Early detection of bone marrow engraftment by amplification of hypervariable DNA regions. Blood. 82:156-160, 1997.
203. Korbling M, Katz R L, Khanna A, Ruifrok A C, Rondon G, Albitar M, Champlin R E, Estrov Z: Hepatocytes and epithelial cells of donor origin in recipients of peripheral blood stem cells. N Engl J Med. 346:738-746, 2002.
204. Bayes-Genis A, Muniz-Diaz E, Catasus L, Arilla M, Rodriguez C, Sierra J, Madoz P J, Cinca J: Cardiac chimerism in recipients of peripheral-blood and bone marrow stem cells. Eur J Heart Fail. 6:399-402, 2004.
205. Tumbar T, Guasch G, Greco V, Blanpain C, Lowry W E, Rendl M, Fuchs E: Defining the epithelial stem cell niche in skin. Science. 303:359-363, 2004.
206. Urbanek K, Cesselli D, Rota M, Nascimbene A, DeAngelis A, Hosoda T, Bearzi C, Boni A, Bolli R, Kajstura J, Anversa P, Leri A: Stem cell niches in the adult mouse heart. Proc Natl Acad Sci USA. 103:9226-9231, 2006.
207. Jan Y N, Jan L Y: Asymmetric cell division. Nature. 392:775-778, 1998.
208. Lu B, Jan L, Jan Y N: Control of cell divisions in the nervous system: symmetry and asymmetry. Annu Rev Neurosci. 23:531-556, 2000.
209. Gotz M, Huttner W B: The cell biology of neurogenesis. Nat Rev Mol Cell Biol. 6:777-788, 2005.
210. Rhyu M S, Jan Y N: Asymmetric distribution of numb protein during division of the sensory organ precursor cell confers distinct fates to daughter cells. Cell. 76:477-491, 1994.
211. Guo M, Jan L Y, Jan Y N: Control of daughter cell fates during asymmetric division: interaction of Numb and Notch. Neuron. 17:27-41, 1996.
212. Berdnik D, Torok T, Gonzalez-Gaitan M, Knoblich J A: The endocytic protein α-adaptin is required for numb-mediated asymmetric cell division in Drosophila. Dev Cell. 3:221-231, 2002.
213. Faubert A, Lessard J, Sauvageau G: Are genetic determinants of asymmetric stem cell division active in hematopoietic stem cells? Oncogene. 23:7247-7255, 2004.
214. Braun K M, Niemann C, Jensen U B, Sundberg J P, Silva-Vargas V, Watt F M: Manipulation of stem cell proliferation and lineage commitment: visualization of label-retaining cells in whole mounts of mouse epidermis. Development. 130:5241-5255, 2003.
215. Baserga R: The biology of cell reproduction. Harvard University Press, 1985.
216. Torella D, Rota M, Nurzynska D, Musso E, Monsen A, Shiraishi I, Zias E, Rosenzweig A, Sussman M A, Urbanek K, Nadal-Ginard B, Kajstura J, Anversa P, Leri A: Cardiac stem cell and myocyte aging, heart failure, and insulin-like growth factor-1 overexpression. Circ Res. 94:514-524, 2004.
217. Houser S R, Leri A, Sussman M A, Anversa P: Nuclear targeting of Akt enhances ventricular function and myocyte contractility. Circ Res. 97:1332-1341, 2005.
218. Ferron L, Capuano V, Deroubaix E, Coulombe A, Renaud J F: Functional and molecular characterization of a T-type Ca(2+) channel during fetal and postnatal rat heart development. J Mol Cell Cardiol. 34:533-546, 2002.
219. Yao A, Spitzer K W, Ito N, Zaniboni M, Lorell B H, Barry W H: The restriction of diffusion of cations at the external surface of cardiac myocytes various between species. Cell Calcium. 22:431-438, 1997.
220. Zaniboni M, Yao A, Barry W H, Musso E, Spitzer K W: Complications associated with rapid caffeine application to cardiac myocytes that are not voltage clamped. J Mol Cell Cardiol. 30:2229-2235, 1998.
221. Stilli D, Sgoifo A, Macchi E, Zaniboni M, DeLasio S, Cerbai E, Mugelli A, Lagrasta C, Olivetti G, Musso E: Myocardial remodeling and arrhythmogenesis in moderate cardiac hypertrophy in rats. Am J Physiol. 280:H142-150, 2001.
222. Rota M, Hosoda T, De Angelis A, Arcarese M L, Esposito G, Rizzi R, Tillmanns J, Tugal D, Musso E, Rimoldi O, Bearzi C, Urbanek K, Anversa P, Leri A, Kajstura J: The young mouse heart is composed of myocytes heterogeneous in age and function. Circ Res. 101:323-325, 2007.
223. Chen S, Lecleider R J: Transforming growth factor-beta-induced differentiation of smooth muscle from a neural crest stem cell line. Circ Res. 94:1195-1202, 2004.
224. Loutzenhiser R, Chilton L, Trottier G: Membrane potential measurements in renal afferent and efferent arterioles: actions of angiotensin II. Am J Physiol. 273:F307-314, 1997.
225. Jackson W F: Ion channels and vascular tone. Hypertension. 35:173-178, 2000.
226. Hill M A, Zou H, Potocnik S J, Meininger G A, Davis M J: Arterior smooth muscle mechanotransduction: Ca(2+) signaling pathways underlying myogenic reactivity. J Appl Physiol. 91:973-983, 2001.
227. Hansen P B, Jensen B L, Andrereasen D, Skott O: Differential expression of T- and L-type voltage dependent calcium channels in renal resistance vessels. Circ Res. 89:630-638, 2001
228. Bruhl T, Heeschen C, Aicher A, Jaddi A S, Haendeler J, Hoffmann J, Schneider M D, Zeiher A M, Dimmeler S, Rossig L: p21Cip1 levels differentially regulate turnover of mature endothelial cells, endothelial progenitor cells, and in vivo neovascularization. Circ Res. 94:686-692, 2004.
229. Anversa P, Olivetti G: Cellular basis of physiological and pathological myocardial growth. Handbook of Physiology. The Cardiovascular System. The Heart. Sect 2 Chapter 2: 75-144, 2002.
230. Wallenstein S, Zucker C L, Fleiss J L: Some statistical methods useful in circulation research. Circ Res. 47:1-9, 1980.
231. Berenson M L, Levine D M, Rindskopf D: Applied statistics. Prentice Hall, Englewood Cliffs. 362-418, 1988.
232. Jeffrey I B, Higgins D G, Culhane A C: Comparison and evaluation of methods for generating differentially expressed gene lists from microarray data. BMC Bioinformatics. 7:359, 2006.
233. Chi X, Chatterjee P K, Wilson W 3rd, Zhang S X, Demayo F J, Schwartz R J: Complex cardiac Nkx2-5 gene expression activated by noggin-sensitive enhancers followed by chamber-specific modules. Proc Natl Acad Sci USA. 102:13490-13495, 2005.
234. Srivastava D, Olson E N: A genetic blueprint for cardiac development. Nature. 207:221-226, 2000.
235. Olson E N: Gene regulatory networks in the evolution and development of the heart. Science. 313:1922-1927, 2006.
236. McKinsey T A, Zhang C L, Olson E N: MEF2: a calcium-dependent regulator of cell division, differentiation and death. Trends Biochem Sci. 27:40-47, 2002.
237. Antos C L, McKinsey T A, Drerlitz M, Hollingsworth L M, Zhang C L, Schreiber K, Rindt H, Gorczynski R J, Olson E N: Dose-dependent blockade to cardiomyocyte hypertrophy by histone deacetylase inhibitors. J Biol Chem. 278:28930-28937, 2003.
238. McKinsey T A, Olson E N: Cardiac histone acetylation-therapeutic opportunities abound. Trends Genet. 20:206-213, 2004.
239. Miano J M: Serum response factor: toggling between disparate programs of gene expression. J Mol Cell Cardiol. 35:577-593, 2003.
240. McDonald O G, Owens G K: Programming smooth muscle plasticity with chromatin dynamics. Circ Res. 100:1428-1441, 2007.
241. McDonald O G, Wamhoff B R, Hoofnagle M H, Owens G K: Control of SRF binding to CArG box chromatin regulates smooth muscle gene expression in vivo. J Clin Invest. 116:36-48, 2006.
242. Qiu P, Li L: Histone acetylation and recruitment of serum responsive factor and CREB-binding protein onto SM22 promoter during SM22 gene expression. Circ Res. 90:858-865, 2002.
243. Cao D, Wang Z, Zhang C L, Oh J, Xing W, Li S, Richardson J A, Wang D Z, Olson E N: Modulation of smooth muscle gene expression by association of histone acetyltransferases and deacetylases with myocardin. Mol Cell Biol. 25:364-376, 2005.
244. Caslini C, Capo-chichi C D, Roland I H, Nicholas E, Yeung A T, Xu X X: Histone modifications silence the GATA transcription factor genes in ovarian cancer. Oncogene. 25:5446-5461, 2006.
245. Fish J E, Matouk C C, Rachlis A, Lin S, Tai S C, D'Abreo C, Marsden P A: The expression of endothelial nitric-oxide synthase is controlled by a cell-specific histone code. J Biol Chem. 280:24824-24838, 2005.
246. Peinado H, Portillo F, Cano A: Transcriptional regulation of cadherins during development and carcinogenesis. Int J Dev Biol. 48:365-375, 2004.
247. Grady W M, Willis J, Guilford P J, Dunbier A K, Toro T T, Lynch H, Wiesner G, Ferguson K, Eng C, Part J G, Kim S J, Markowitz S: Methylation of the CDH1 promoter as the second genetic hit in hereditary diffuse gastric cancer. Nat Genet. 26:16-17, 2000.
248. Graff J R, Gabrielson E, Fujii H, Baylin S B: Methylation patterns of the E-cadherin 5′ CpG island are unstable and reflect the dynamic, heterogeneous loss of E-cadherin expression during metastatic progression: J Biol Chem. 28: 2727-2732, 2000.
249. Leri A, Claudio P P, Li Q, Wang X, Reiss K, Wang S, Malhotra A, Kajstura J, Anversa P: Stretch-mediated release of angiotensin II induces myocyte apoptosis by activating p53 that enhances the local renin-angiotensin system and decreases the Bcl-2-to-Bax protein ratio in the cell. J Clin Invest. 101:1326-1342, 1998.
250. Leri A, Liu Y, Wang X, Kajstura J, Malhotra A, Meggs L G, Anversa P: Overexpression of insulin-like growth factor-1 attenuates the myocyte renin-angiotensin system in transgenic mice. Circ Res. 84:752-762, 1999.
251. Boni A, Nascimbene A, Urbanek K, Delucchi F, Gonzalez A, Siggins R, Amano K, Yasuzawa-Amano S, Ojaimi C, Rota M, Hosoda T, Anversa P, Kajstura J, Leri A: Notch1 receptor enhances myocyte differentiation of cardiac progenitor cells and myocardial regeneration after infarction. Submitted, 2007.
252. Fraga M F, Esteller M: Epigenetics and aging: the targets and the marks. Trends Genet. 23:413-418, 2007.
253. Blasco, M A: The epigenetic regulation of mammalian telomeres. Nat Rev Genet. 8:299-309, 2007.
254. Cheng W, Reiss K, Li P, Chun M, Kajstura J, Olivetti G, Anversa P: Aging does not affect the activation of the myocyte insulin-like growth factor-1 autocrine system after infarction and ventricular failure in Fischer 344 rats. Circ Res. 78:536-546, 1996.
255. Chimenti C, Kajstura J, Torella D, Urbanek K, Heleniak H, Colussi C, Di Meglio F, Nadal-Ginard B, Frustaci A, Leri A, Maseri A, Anversa P: Senescence and death of primitive cells and myocytes lead to premature cardiac aging and heart failure. Circ Res. 93:604-613, 2003.
256. Losordo D W, Schatz R A, White C J, Udelson J E, Veereshwarayya V, Durgin M, Poh K K, Weinstein R, Kearney M, Chaudhry M, Burg A, Eaton L, Heyd L, Thorne T, Shturman L, Hoffmeister P, Story K, Zak V, Dowling D, Traverse J H, Olson R E, Flanagan J, Sodano D, Murayama T, Kawamoto A, Kusano K F, Wollins J, Welt F, Shah P, Soukas P, Asahara T, Henry T D: Intramyocardial transplantation of autologous CD34+ stem cells for intractable angina: a phase I/IIa double-blind, randomized controlled trial. Circulation. 115:3165-3172, 2007.
257. Post W S, Goldschmidt-Clermont P J, Wilhide C C, Heldman A W, Sussman M S, Ouyang P, Milliken E E, Issa J P: Methylation of the estrogen receptor gene is associated with aging and atherosclerosis in the cardiovascular system. Cardiovascular Res. 43:985-991, 1999.
258. Fricker J: Heart disease linked to oestrogen-receptor gene methylation. Mol Med. Today. 5:505-506, 1999.
259. Barik S: The thrill can kill: murder by methylation. Mol Pharmacol. 71:1203-1205, 2007.
260. Shumaker D K, Dechat T, Kohlmaier A, Adam S A, Bozovsky M R, Erdos M R, Eriksson M, Goldman A E, Khuon S, Collins F S, Jenuwein T, Goldman R D: Mutant nuclear lamin A leads to progressive alterations of epigenetic control in premature aging. Proc Natl Acad Sci USA. 103:8703-8708, 2006.
261. Sommer M, Poliak N, Upadhyay S, Ratovitski E, Nelkin B D, Donehower L A, Sidransky D: DeltaNp63alpha overexpression induces downregulation of Sirt1 and an accelerated aging phenotype in the mouse. Cell Cycle. 5:2005-2011, 2006.
262. Sasaki T, Maier B, Bartke A, Scrable H: Progressive loss of SIRT1 with cell cycle withdrawal. Aging Cell. 5:413-422, 2006.
263. Vaquero A, Sternglanz R, Reinbert D: NAD+-dependent deacetylation of H4 lysine 16 by class III HDACs. Oncogene. 26:5505-5520, 2007.
264. Barley N A, Liu L, Chehab, N H, Mansfield K, Harris K G, Halazonetis T D, Berger S L: Acetylation of p53 activates transcription through recruitment of coactivators/histone acetyltransferases. Mol Cell. 8:1243-1254, 2001.
265. Gu W, Luo J, Brooks C L, Nikolaev A Y, Li M: Dynamics of the p53 acetylation pathway. Novartis Found Symp. 259:97-205, 2004.
266. Saunders L R, Verdin E: Sirtuins: critical regulators at the crossroads between cancer and aging. Oncogene. 26:5489-5504, 2007.
267. Yoo C B, Jones P A: Epigenetic therapy of cancer: past, present and future. Nat Rev Discov. 5:37-50, 2006.
268. Sigalotti L, Fratta E, Coral S, Cortini E, Covre A, Nocolay H J, Anzalone L, Pezzani L, Di Giacomo A M, Fonzatti E, Colizzi F, Altomonte M, Calabro L, Maio M: Epigenetic drugs as pleiotropic agents in cancer treatment: biomolecular aspects and clinical application. J Cell Physiol. 212:330-344, 2007.
269. Xu W S, Parmagiani R B, Marks P A: Histone deacetylase inhibitors: molecular mechanisms of action. Oncogene. 26:5541-5552, 2007.
270. Yoshida M, Horinouchi S: Trichostatin and leptomycin. Inhibition of histone deacetylation and signal-dependent nuclear export. Ann NY Acad. Sci. 886:23-36, 1999.
271. Kong Y, Tannous P, Lu G, Berenji K, Rothermel B A, Olsen E N, Hill J A: Suppression of class I and II histone deacetylases blunts pressure-overload cardiac hypertrophy. Circulation. 113:2579-88, 2006.
272. Mai A, Massa S, Pezzi R, Valente S, Loidl P, Brosch G: Synthesis and biological evaluation of 2-, 3-, and 4-acylaminocinnamyl-N-hydroxyamides as novel synthetic HDAC inhibitors. Med. Chem. 1:245-254, 2005.
273. Gonzalez A, Rota M, Nurzynska D, Misao Y, Tillmanns J, Ojaimi C, Padin-iruegas M E, Muller P, Esposito G, Bearzi C, Vitale S, Buddhadeb D, Math S, Baker M, Hintze T H, Bolli R, Urbanek K, Hosoda T, Anversa P, Kajstura, Leri A: Activation of cardiac progenitor cells reverses the failing heart senescent phenotype and prolongs lifespan. Submitted, 2007.
274. Tillmanns J, Rota M, Hosoda T, Rotatori F, DeAngelis A, Amano K, Amano S, LeCapitaine N, Esposito G, Loredo M, Misao Y, Vitale S, Bearzi C, Urbanek K, Bolli R, Leri A, Kajstura J, Anversa P: Formation of large coronary arteries by cardiac stem cells: a biological bypass. Proc Natl Acad Sci USA. In revision, 2007.
275. Rota M, LeCapitaine N, Hosoda T, Boni A, DeAngelis A, Padin-Iruegas M E, Esposito G, Vitale S, Urbanek K, Casarsa C, Giorgio M, Luscher T F, Pelicci P G, Anversa P, Leri A, Kajstura J: Diabetes promotes cardiac stem cell aging and heart failure, which are prevented by deletion of the p66shc gene. Circ Res. 99:42-52, 2006.

1. A composition, comprising a histone deacetylase (HDAC) inhibitor and one or more types of human progenitor cells.

2. The composition of claim 1, wherein the one or more types of human progenitor cells are selected from the group consisting of human vascular progenitor cells, human myocyte progenitor cells, human bone marrow progenitor cells, and combinations thereof.

3. The composition of claim 2, wherein the human vascular progenitor cells are lineage negative, c-kit positive, and KDR positive.

4. The composition of claim 2, wherein the human myocyte progenitor cells are lineage negative, c-kit positive, and KDR negative.

5. The composition of claim 2, wherein the human bone marrow progenitor cells are lineage negative and c-kit positive.

6. The composition of claim 1, wherein said HDAC inhibitor targets class I or class II HDAC enzymes.

7. The composition of claim 6, wherein said HDAC inhibitor is trichostatin A, MS27-275, or MC1568.

8. The composition of claim 2, wherein said HDAC inhibitor is an siRNA molecule targeted to a class I or class II HDAC enzyme.

9. The composition of claim 8, wherein said siRNA molecule is targeted to a HDAC enzyme selected from the group consisting of HDAC4, HDAC5, HDAC7, and HDAC 9.

10. The composition of claim 8, wherein the one or more types of human progenitor cells express said siRNA molecule.

11. A method of enhancing progenitor cell proliferation comprising:
exposing human adult progenitor cells to one or more HDAC inhibitors, wherein said progenitor cells exhibit enhanced proliferation as compared to progenitor cells not exposed to the one or more HDAC inhibitors.

12. The method of claim 11, wherein the one or more HDAC inhibitors target a class I and/or class II HDAC enzyme.

13. The method of claim 11, wherein said human adult progenitor cells are selected from the group consisting of human vascular progenitor cells, human myocyte progenitor cells, human bone marrow progenitor cells, and combinations thereof.

14. A method of enhancing progenitor cell differentiation comprising:
exposing human adult progenitor cells to one or more HDAC inhibitors, wherein said progenitor cells exhibit enhanced differentiation as compared to progenitor cells not exposed to the one or more HDAC inhibitors.

15. The method of claim 14, wherein the one or more HDAC inhibitors target a class I and/or class II HDAC enzyme

16. The method of claim 14, wherein said human adult progenitor cells are selected from the group consisting of human vascular progenitor cells, human myocyte progenitor cells, human bone marrow progenitor cells, and combinations thereof.

17. A method of restoring progenitor cell function to aged adult progenitor cells comprising:
exposing said aged progenitor cells to one or more HDAC inhibitors, wherein said progenitor cells exhibit increased expression of at least one stem cell related gene as compared to aged progenitor cells not exposed to the one or more HDAC inhibitors.

18. The method of claim 17, wherein said stem cell related gene is Oct4 or Nanog.

19. The method of claim 17, wherein the aged progenitor cells are isolated from a subject suffering from heart failure.

20. A method of treating heart failure in a subject in need thereof comprising:
(a) isolating adult progenitor cells from a tissue specimen from the subject;
(b) exposing said isolated progenitor cells to one or more HDAC inhibitors; and
(c) administering said treated progenitor cells to the subject's heart, wherein said progenitor cells generate new coronary vessels and myocardium, thereby improving cardiac function.

21. The method of claim 20, wherein said adult progenitor cells are selected from the group consisting of human vascular progenitor cells, human myocyte progenitor cells, human bone marrow progenitor cells, and combinations thereof.

22. The method of claim 20, wherein the one or more HDAC inhibitors target a class I and/or class II HDAC enzyme.

23. The method of claim 20, wherein at least one symptom of heart failure is reduced in the subject following administration of the treated progenitor cells.


SEQUENCE LISTING (2 sequences)

SEQ ID NO: 1 - DNA, 26 bp, Homo sapiens
gagaagctcttccttcctttgcactg

SEQ ID NO: 2 - DNA, 26 bp, Homo sapiens
ttcgggtatttctctctgtgcatggc